(19) United States
(12) Patent Application Publication
(10) Pub. No.: US 2026/0148801 A1
(43) Pub. Date: May 28, 2026

(54) EPIGENETIC CLOCK

(71) Applicant: Buck Institute for Research on Aging, Novato, CA (US)

(72) Inventors: Alan Tomusiak, Novato, CA (US); Eric Verdin, Novato, CA (US)

(21) Appl. No.: 19/120,747

(22) PCT Filed: Oct. 13, 2023
(86) PCT No.: PCT/US2023/035141

§ 371 Date: Apr. 11, 2025

Related U.S. Application Data
(60) Provisional application No. 63/415,947, filed on Oct. 13, 2022.

(51) Int. Cl. G16B 20/20; C12Q 1/68; G16B 40/20; G16H 10/60

(52) U.S. Cl. CPC G16B 20/20; C12Q 1/68; G16B 40/20; G16H 10/60; C12Q 2600/154

(57) ABSTRACT

Provided herein are methods and compositions for an epigenetic clock comprising differentiation-independent methylation sites to asses biological age of a human subject.

### CROSS-REFERENCE OT RELATED APPLICATIONS

This application claims priority to U.S. provisional application No. 63/415,947, filed Oct. 13, 2022, which is herein incorporated by reference in its entirety for all purposes.

### BACKGROUND OF THE INVENTION

“Clocks” based on machine-learning models of changing DNA methylation patterns have recently been developed for detecting aging-associated changes associated with lifespan. However, how these epigenetic clocks operate and what aging-associated biology they are tracking has been unclear.

### BRIEF SUMMARY OF THE INVENTION

In one aspect, the disclosure provides an epigenetic clock that is not skewed by the differentiation state of the cells within the sample being analyzed to assess the biological age of a human subject. Thus, for example, in some embodiments, methylation patterns of CpG sites are determined in various subpopulations of cells, e.g., immune cells such as T cells, and evaluated for changes in methylation patterns associated with differentiation. Those sites that undergo differentiation-dependent methylation can be removed from a database of CpG sites that are associated with aging. Accordingly, the methylation sites employed in the present disclosure do not change in differentiating cells of the same lineage. Further, as described herein, machine learning techniques can be employed to predict age from the differentiation-neutral CpG cites.

Thus, in on aspect, provided herein is a two-step procedure for generating a DNA methylation clock panel comprising differentiation-independent CpG sites predictive for aging-associated changes in lifespan, wherein the CpG sites are selected, e.g., via clastic net regression, with a subsequent prediction step performed on the selected CpGs using a deep learning algorithm. This additional step of refinement provides stronger predictive accuracy for age and higher precision.

### BRIEF DESCRIPTION OF THE DRAWINGS

FIG. 1 provides a graph depicting a clock that exhibits no shift in age.

FIG. 2 provides a graph depicting that the clock of FIG. 1 does not shift with different stage of T cell differentiation. (CD8 T cells) The new clock (arrow) is compared to other clocks.

FIG. 3 provides a graph showing the accuracy of subsets of methylation markers as described herein.

FIG. 4 illustrates three steps for prediction algorithm creation.

FIG. 5 illustrates that clock-predicted ages of nine donors show no differences in age prediction between ten immune cell types.

FIG. 6. The clock-predicted ages of six donors do not show differences in predicted ages between CD 8+ naïve cells and CD8+ effector cells. IntrinClock predicted ages of seven donors do not show predicted ages between CD4+ naïve cells and CD4+ memory cells.

FIG. 7. Clock-predicted ages of individuals do not vary depending on blood proportions of CD8+ effector memory, CD4+ central memory, class-switched B cells, CD16+CD56dim NK cells, or classical monocytes.

FIG. 8. Repeated elastic net machine learning (b) lowers mean absolute error of age prediction by an average of three months compared to using only one round of elastic net (a).

### DETAILED DESCRIPTION OF THE INVENTION

### Terminology

As used herein, the singular form “a”, “an”, and “the” include plural references unless the context dictates otherwise. For example, the term “a nucleic acid” includes a plurality of nucleic acids.

The term “epigenetic” as used herein means relating to, being, or involving a chemical modification of the DNA molecule. Such chemical modifications include the addition or removal of a methyl group on cytosine residues, e.g. that occur in a CpG dinucleotide.

As used herein, “methylation status” refers to the presence of methyl groups at a particular DNA sequence. In some embodiments, methylation of DNA refers to the presence or absence of 5-methylcytosine (5-mC) at one or more CpG dinucleotides in a DNA sequence. Methylation states at one or more particular methylation sites within a DNA sequence include “unmethylated,” “fully-methylated,” “hypomethylated” or “hypermethylaed”. “Hypomethylated” and “hypermethylated” refer to the average methylation state corresponding to an increased presence of 5-mC at one or a plurality of CpG dinucleotides within a methylation target sequence.

The term “methylation site” as used herein refers to a CpG position that is potentially methylated. The CpG containing nucleic acid may be present, e.g., in a CpG island, a CpG doublet, a promoter, an intron, or an exon of gene. A panel of methylation sites as described herein comprises members that undergo changes in methylation status associated with age-related health outcomes and mortality. Accordingly, an individual may have a biological agent that is greater or lower than chronological age as measured using a differentiation-independent panel of methylation states as described herein.

A “differentiation-independent” methylation site as used herein refers to a methylation site that does not exhibit varied methylation patterns among cells of the same lineage at varying stages of differentiation.

A “methylation profile” as used herein refers to the methylation status (degree of methylation) of each methylation site evaluated in a panel of methylation sites for which methylation correlates with age.

The term “biological age” as used herein refers to age as determined relative to age-related health outcomes associated with methylation status of a set of differentiation-independent methylation sites as described herein that are not solely based on chronological age of an individual. In some embodiments, individuals of the same chronological age may have marked susceptibility to age related diseases, which can influence longevity. In order to provide a reference panel of methylation markers to determine a scale for health outcomes associated with methylation status, a reference population of subjects can be used. Illustrative control populations include, but are not limited to healthy individuals; individuals who do not have cancer: symptoms of a severe age-associated diseases such as dementia, e.g., Alzheimer's or other neurodegenerative diseases such as Parkinson's disease; stroke, ischemic heart disease, heart failure or other disease associated with age.

As used herein, the term “about” means that the item, parameter or term so qualified encompasses a range of plus or minus twenty percent, or plus or minus ten percent, above and below the value of the stated item, parameter or term. Accordingly, unless indicated to the contrary, various numerical parameters set forth in the specification and attached claims are approximations that may vary depending upon the desired properties sought to be obtained by the present invention.

### Generating a Panel of Differentiation-Independent Methylation Markers

Many DNA methylation data sets are publicly available (e.g., GEO e, NCBI). In some embodiments, commercially available tools for querying methylation status of a vast number of methylation sites include array chips, such as the 450K or MethylationEPIC array (over 850,000 methylations sites) chips available from Illumina, which can be used to interrogate bisulfite-treated samples to determine methylation status to create a database of methylation sites along with relevant information about the samples, including, for example, information such as age of subject, health status of the person from whom the sample was obtained; the tissue and/or cell type from which the sample was derived. In some embodiments, datasets of about 10,000 or 20,000 or greater can be employed.

In the present disclosure, CpG sites are further characterized to identify sites that are differentiation-dependent so that those sites can be excluded from datasets used to determine a biological age as described herein. This is performed by characterizing methylation in cells of the same lineage at various differentiation stages. In some embodiments, differentiation-dependent CpG sites that undergo changes in methylation during skeletal muscle differentiation and/or immune cell differentiation, and/or skin cell differentiation are identified in order to be excluded from the biological age determination analysis. In some embodiments, CpG sites that exhibit changed in methylation associated with differentiation of T cells are identified. Thus, in some embodiments, subsets of T cells, e.g., cytotoxic T cells, corresponding to naïve T cells, central memory T cells, and/or effector memory T cells can be evaluated to determine differentially methylated sites associated with the different subsets. In some embodiments, cells in the myeloid lineage are evaluated. In some embodiments, B cell lineage cells are evaluated.

Identification of CpG Sites Associated with Differentiation

Dimensionality reduction (e.g., using UMAP or principal component analysis, or an alternative methodology) can then be performed on the DNA methylation profiles in the lineage of cells along the differentiation pathway. Following dimensionality reduction, one of the dimension is used as an axis of differentiation for fitting linear or nonlinear models to each individual DNA methylation site (CpG) and the relationship with the differentiation axis. Once this is completed, p-values are generated using any appropriate statistical testing method (such as the Benjamini-Hockberg adjustment for a p-value calculation) to predict the probability of any individual CpG being associated with differentiation. All CpG values with p-values below a certain threshold are then discarded prior to machine learning model training. In some embodiments, the threshold is 0.35 or greater in some embodiments, the threshold is about 0.4 or greater. In some embodiments, the threshold is about 0.5 or greater. In some embodiments, the threshold is about 0.6 or greater. Thus, for example, a threshold of 0.6 that each CpG has a less than 40% chance of being associated with differentiation.

All CpG sites that are identified as differentiation-associated can then be excluded from CpG sites to be evaluated for biological age. The remaining markers (differentiation-independent methylation sites) can then be used for machine learning model training.

In some embodiments, age associated with each CpG value can be transformed into a value using an approach described by Horvath, Genome Biology 14: 3156 (2013) to fulfill certain desirable statistical properties, for the subsequent machine learning steps for training.

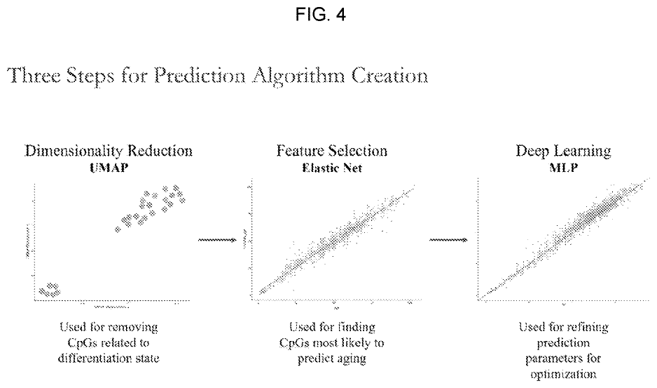

CpGs most likely to predict aging can the be selected. Selection is usually performed using a feature selection algorithm, which can provide CpGs with have a strong predictive power when used together. A strong correlation is often considered to be a correlation between prediction and true value with an R² above 0.7. In some embodiments, elastic net regression is employed. Additional feature selection algorithms are discussed, e.g., by Li et al. PLOS Comput Biol 18(8):e1009938, 2022. Deep Learning, e.g., MLP, can additionally be used for refining prediction parameters. A schematic of steps of generating an epigenetic clock as described herein is provided in FIG. 4.

### Determining Biological Age

The methods described herein are additionally based, in part, on the identification of a set of differentiation-independent CPG sites (see, e.g., Table 1) for which methylation status correlates with age-related changes in life expectancy that provides a “clock” to calculate biological age of an individual. In Table 1, each CpG value is based on methylation of a cytosine (as shown in the “Forward_Sequence” column), which includes sufficient sequence information to allow identification of which cytosine in the genome is queried.

In some embodiments, methylation status is evaluated for at least 150 of the CpG sites set forth in Table 1. In other embodiments, methylation status is evaluated for at least 200 CpG sites set forth in Table 1. In still other embodiments, methylation status is evaluated for at least 250 CpG sites set forth in Table 1 or at least 300 CpG sites. In further embodiments, methylation status is evaluated for at least 350 sites set forth in Table 1, or at least 400, at least 450, at least 500, or all 537 sites set forth in Table 1. One of skill understands that many subsets of the 537 panel can be employed for analyzing methylation status in a sample.

This analysis can be performed on DNA isolated from a sample obtained from the subject, such as a swab, blood sample, or any other sample that provides genomic DNA that can be queried. Methylation status of genomic DNA obtained from sample can be determined using well known methodology, typically based on sodium bisulfite conversion of genomic DNA (involving deamination of unmodified cytosines to uracil, leaving methylated cytosines unchanged) from a sample to be evaluated to distinguish and detect unmethylated versus methylated cytosines. Analysis can be performed by interrogation of an array comprising a probe specific for a methylated and a probe specific for the unmethylated from of the site (see, e.g., Illumina methylation arrays) or by sequence analysis. In other embodiments, DNA methylation can be evaluated and quantified using methylation-sensitive restriction enzyme-based approaches, e.g., where methylated sites are known to include a methylation-sensitive restriction enzyme sites, affinity enrichment-based approaches, methylation-sensitive PCR or ligase chain reaction, or any of a number of other approaches. See, e.g., Yong et al, Epigenetics Chromatin 9:26, 20216; Bock et al, Nat Rev Genet 13:705-19, 2012; Laird et al, Nat Rev Genet 11:191-203, 2010; Adusumalli et al, Brief Bioinform. 163:369-79, 2015; Barros-Silva et al., Genes (Basel) 9:429, 2018; Wreczycka et al., J Biotechnol. 261:105-15, 2017 for a discussion of illustrative methods to determine methylation status.

In some embodiments, deep learning methods employing neural networks are employed for predicting biological age. For example, a Python or R package such as tensorflow or keras can be imported to predict age using a pre-built model that was constructed based on age.

The results of the methylation analysis can be normalized and quantified and then utilized to generate predictions of chronological age utilizing a feature selection regression algorithm such as Elastic Net. In such an analysis, a beta value can be derived by measuring the intensities of signal generated by the probe to detect the methylated site vs the signal generated by the probe to detect the unmethylated site. Thus, in the context of array analysis such as Illumina methylation array analysis), the term “beta-value” refers to computation of methylation level at a CG position derived by normalization and quantification of Illumina methylations status arrays, such as Illumina 450K or EPIC arrays using the ratio of intensities between methylated and unmethylated probes and the formula: beta value=methylated C intensity/(methylated C intensity+unmethylated C intensity) between 0 and 1 with 0 being fully unmethylated and 1 being fully methylated.

After assessing methylation status of the CpG sites as described herein, e.g., in Table 1, a biological age can be calculated. In Table 1, each CpG value is based on methylation of a cytosine (as shown in the “Forward_Sequence” column, which includes sequences upstream (S′) of the methylated cytosine in question and allow identification of which cytosine in the genome is queried. When using the parameters discovered by an Elastic Net algorithm to perform predictions based on linear regression, each beta value is multiplied by a coefficient (illustrated by “Value” column in Table 1). Once all of the CpG beta values are multiplied their coefficients, the results are added together. An intercept term -.4915 is then added.

In some embodiments, repeated elastic net machine learning is performed for the prediction of biological age.

### Computer-Implemented Methods

In some embodiments, a database comprising reference values for methylation status of differentiation-independent CpG loci is generated. Accordingly, aspects of the invention provide systems and methods for the use and development of a database. In some approaches, the database is used in combination with an algorithm that enables generation of new reference profiles selected based on characteristics of an individual subject.

Methods of the invention may be implemented using a computer-based system. Accordingly, a related embodiments includes a tangible computer-readable medium comprising computer-readable code that, when executed by a compute, causes the computer to perform operations including: receiving information corresponding to methylation levels at a set of methylation markers, e.g., methylation sites set forth in Table 1; and determining a biological age by applying a statistical prediction algorithm to methylation date from the set of methylation markers; and then determining the biological age, e.g., using a weighted average of the methylations levels of the markers, e.g., the 537 markers set for in Table 1 or a subset thereof as described herein.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

A computer system can include a plurality of the same components or subsystems, e.g., connected together by external interface, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

The databases may be provided in a variety of forms or media to facilitate their use. “Media” refers to a manufacture that contains the expression information of the present invention. The databases of the present invention can be recorded on computer readable media, e.g. any medium that can be read and accessed directly by a computer (e.g., an internet database). Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable media can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### Kits and Compositions

Also provided herein are kits and compositions for determining the biological age of a human subject. In some embodiments, a kit comprises an array that comprises probes to query at least 200, at least 300, at least 400, or at least 500 methylation sites set forth in Table 1 with the proviso that the array is not an Illumina array. In some embodiments, the array contains probes for the interrogation of 1,000 or fewer sites. In some embodiments, a kit further comprises computer software to determine biological age.

### Technical Section Illustrating Generation of an Immune Cell Differentiation-Independent Epigenetic Clock.

List of DNA methylations datasets are publicly available. For this analysis, methylation status of methylation sites was performed using Illumina 450K or the MethylationEPIC Array chips. A database was generated containing information re samples analyzed and their corresponding DNA methylation levels (ranging from 0 to 1; 0=fully hypomethylated, 1=fully hypermethylated) at each of 450,000 assayed locations on the genome (CpG sites) by both the 450K and MethylationEPIC chips. A database storing metadata for each sample was also generated, which specifically tracked: 1) Sample ID; 2) Health status of the person from whom the sample was obtained; 3) The tissue from which the sample was derived; 4) The cell type from which the sample was derived; 5) The first author of the most recent publication containing data from the dataset; and the year of the publication; 6); the chip used to evaluate the sample; and 7) the age of the individual from whom the sample was obtained. The databased contained approximately 14,000 samples from 61 datasets.

All CpGs sites that had missing values in more than 10% of all sample were removed and all samples that had missing values in more than 10% of CpG sites were removed. This resulted in a database of about 350,000 CpG sites assessed on 12,000 samples remaining from 56 datasets.

A list of CpG sites that were associated with differentiation of immune cells, e.g., T cells, was then generated. Blood samples from seven donors of varying ages were obtained. Peripheral blood mononuclear cells (PBMCs) were isolated. A T cell population was obtained using a commercial EasySep™ Human T Cell Enrichment Kit. Four distinct T cell subpopulations that exist along a differentiation trajectory (cytotoxic naïve T cells, cytotoxic central memory T cells, cytotoxic effector memory T cells, and cytotoxic effector memory RA+ (TEMRA cells) were then labeled using fluorophore-conjugated antibodies, specifically CD3, CD8, CD4, CD28, CD45RO, and a marker that separates live from dead cells. Fluorescence-activated cell sorting (FACS) was performed to separate the four subtypes into individual tubes for each of the seven donors followed by isolation of DNA (Zymo Quick-DNA kit) from each tube, which provided 28 samples. DNA quality was verified by spectrophotometry to ensure sufficient quantity and quality for DNA methylation assessment. Bisulfite conversion and DNA methylation assessment using an Illumina Methylation EPIC array chip was performed by a commercial vendor.

Data was received as a file that tracked methylated and unmethylated probe intensities for each CpG site. The data were converted to beta values for each locus, which provided a single value tracking the methylation at a locus. Conversion was performed and the data pre-processed using Illumina's preprocessing methodology using the minfi R package. As some sites had missing values for certain samples, we therefore used an imputation algorithm (specifically, the K-nearest neighbor technique, implemented in the R package impute) to predict and fill in the missing values. This resulted in a complete data set, with samples linked to methylation values ranging from 0-1 at distinct CpG sites.

Identification of CpG Sites Associated with Differentiation

Dimensionality reduction (using the UMAP algorithm implemented in the R library umap) was performed on 28 samples (see, above). It was observed via plotting that each of the two remaining dimensions correlated strongly with the differentiation state of the cells assayed. In view of this finding, the value at which each sample was located along the X axis was extracted and used as a proxy for measuring differentiation state. A linear model (using the R package limma) for each of the CpG sites was then fit to the differentiation state proxy value, and (using the same package) the probability assess of the two being associated, either in a positive or negative direction. All CpG sites with a 40% or greater probability of being associated with differentiation were separately identified on a list of “differentiation-associated CpGs.”

All CpG sites that were on the “differentiation-associated CpGs list” were removed from the 12,000 samples of 350,000 CpGs, which resulted in about 84,000 CpG sites. All samples present in the 12,000 samples that were from individuals with any measured disease were removed, leaving about 9,000 samples. The remaining datasets containing the 9,000 samples into a training set (of 3,000 samples) and a test set (of 4,000 samples) and performed imputation (as before, using the impute R package) to provide missing values. Lastly, age was transformed into a value using an approach described by (Horvath. 2013, supra) to fulfill certain desirable statistical properties, improving the subsequent machine learning steps.

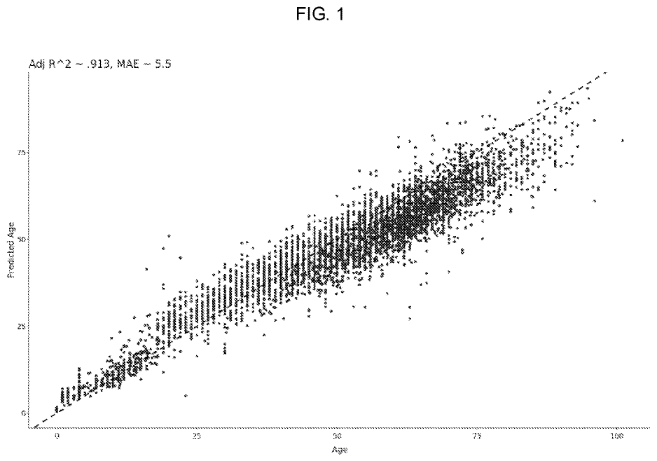

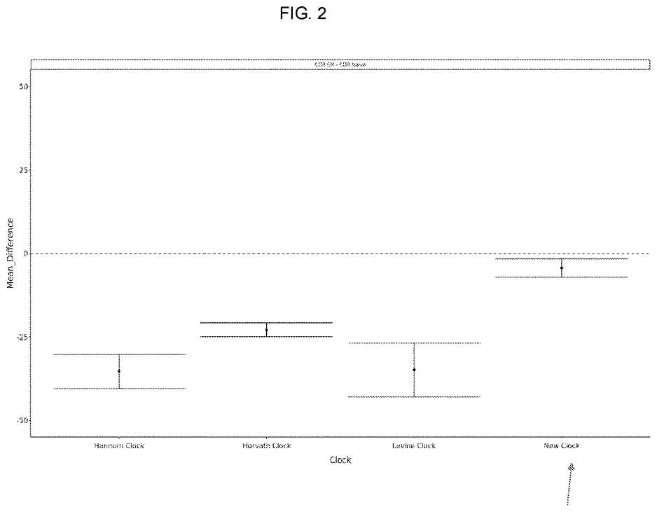

A machine-learning mode was then constructed. An Elastic Net feature selection and regularization algorithm (implemented via the glmnet package in R) was performed on our training set to identify a list of 537 CpG sites with strong age predictive power. These sites were extracted (removing the remaining ˜83,500) a Multi-Layer Perceptron (MLP) deep learning approach (implemented via the keras package in Python) was employed to create a multi-laver model that could be used to predict age given the specified 537 CpG sites. Predictive power was assessed by testing our model on the test set, which determined that age could accurately be predicted within ˜5 years. External datasets that assayed cytotoxic effector memory and naïve cells from the same blood donor were queried to ensure that the model was truly differentiation-independent. This identified that there was no shift in age with this clock (FIG. 1) with no skew related to T cell differentiation (FIG. 2) in contrast to previously generated clocks, which saw a large shift. The model was also tested on various cell states in a helper T cell lineage and showed no changes (data not shown), which indicated that differentiation markers were removed that were not unique to cytotoxic T cells.

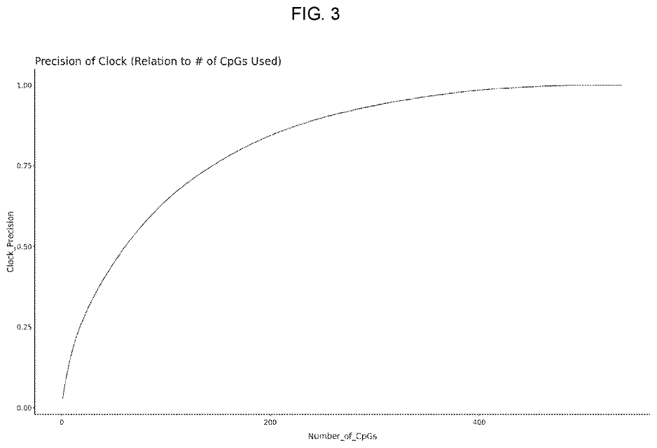

An analysis of the cumulative importance of the number of CpG sites employed for the prediction accuracy of the clock was also performed. An illustrative graph is provided in FIG. 3. The analysis of this clock showed that about 172 sites provided about 80% prediction accuracy. 252 (about half) of the sites provided about 90% prediction accuracy, and 323 sites provided 95% prediction accuracy.

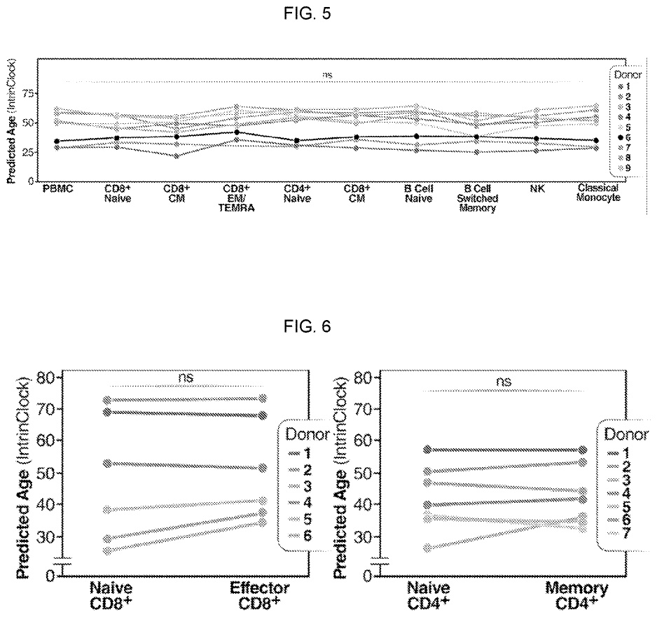

Additional analyses of biological predicted age were performed. CD8+ naive (CD8+CD28+CD45RO−), CD8+CM (CD8+CD28+CD45RO+), CD8+ combined EM/TEMRA (CD8+CD28−), CD4+ naive (CD4+CD28+CD45RO−), CD4+CM (CD4+CD28+CD45RO+), B-cell naive (CD3−CD19+CD27-IgD+), class-switched B cells (CD3−CD19+CD27+IgD−), CD16+CD56dim NK cells (CD3−CD19−CD56dimCD16+), classical monocytes (CD3−CD19−HLADR+CD14+CD16dim), and whole-peripheral blood mononuclear cell (PBMC) samples were sorted using FACS from a separate set of nine donors (five women, four men) aged 30-68 and collected DNA for methylation analysis. FIG. 5 provides data showing that the differentiation-independent DNA methylation clock predicted ages of nine donors and showed show no differences in age prediction between ten immune cell types. Each line represents a separate donor.

Two publicly available datasets of sorted naïve CD8+, memory CD8+, naïve CD4+, and memory CD4+ T cells were also employed to determine whether our differentiation-independent clock predicts different ages between two different immune cell subsets. FIG. 6 provides data illustrating that our differentiation-independent clock-predicted ages of six donors did not show differences in predicted ages between CD8+ naïve cells and CD8+ effector cells (FIG. 6, left panel). Further, predicted ages of seven donors also did not show differences in predicted ages between CD4+ naïve cells and CD4+ memory cells (FIG. 6, right panel). This reinforces that this differentiation-independent clock robustly predicts even ages across multiple cell subsets. Each line represents a separate donor.

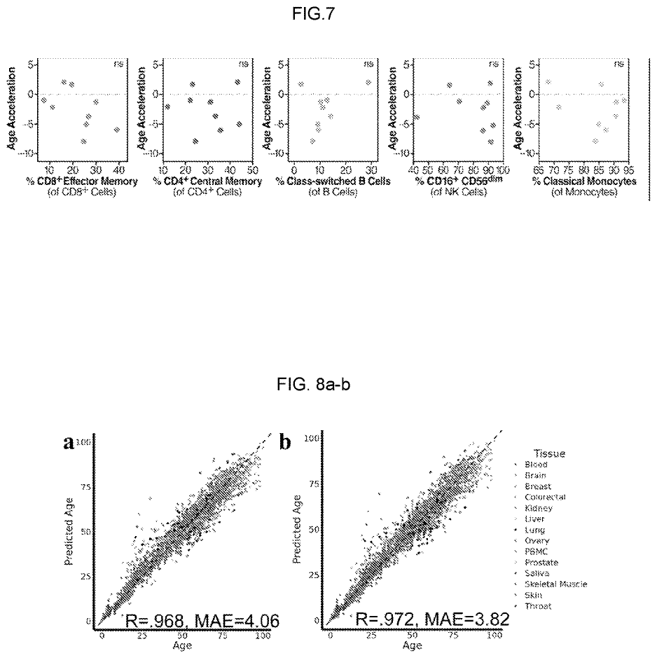

We also utilized high-dimensional flow cytometry to predict the blood cell proportions of five cell subsets from nine donors. The results, provided in FIG. 7, demonstrate that our differentiation-independent clock-predicted ages of individuals did not vary depending on blood proportions of CD8+ effector memory, CD4+ central memory, class-switched B cells, CD16+CD56dim NK cells, or classical monocytes. This indicates that the clock is resilient to changes in blood cell composition.

We also sought to determine whether the innovation described wherein the elastic net algorithm is run twice is successful in reducing age prediction error. We thus compared the age prediction error of running the elastic net machine learning algorithm once vs. two times using the same training set (FIG. 8a-b). We observe d that repeated elastic net machine learning (panel b) lowered mean absolute error of age prediction by an average of three months compared to using only one round of elastic net (panel a). The R package glmnet was utilized to perform the elastic net model training. More specifically, an elastic net model using glmnet was used to develop the differentiation-independent clock, with alpha value set at 0.5. Once the first model (panel a) was generated, the training data were a subset of only those CpGs with non-zero coefficients, which were used for training the final model (panel b, repeated elastic net). The regularization parameter for both elastic net models was generated using cross-validation (cv.glmnet( ) function) with ten folds.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference

TABLE 1

| CpG | Forward_Sequence | Value | Direction |
|---|---|---|---|
| cg00025981 | CCCCTTGGGACAATGCGTAGGGGACCTCCGCGTCCCCGACACCCGACTGGGACAC | 0.04060095 | Hypermethylated with Age |
| | GGCCG[CG]GGCTCCTTCGTCCCTCACCGCCAGCCAGGGAGGCTCTGCATGCCCA | | |
| | CGTCCACTTCACAG | | |
| cg00055555 | AGCAGGGAGCAACAGACAAGCCCAAATTGCTGTGTTTAAAGGAGCAGGGCTGTCT | −0.0125429 | Hypomethylated with Age |
| | GTTTG[CG]TGGGGCTGTCTCCCTGTAATGAGAACCACCGCTGAGAGCTGTTTAG | | |
| | ACAAACGGGCTCAG | | |
| cg00225576 | AGTCCGGGGTCGCCGCAGCCCGGGAGGAGTGTCTGGTCTCCGGCCTGCCTGTGCT | −0.00014186 | Hypomethylated with Age |
| | GTCCC[CG]CGCCCTGTCCACTGGACTCCCGAGACCCTTGGAACCCAGGTAACCC | | |
| | GGGGGGGGACTCCC | | |
| cg00277334 | GTGTGAGGGAGGGGGAACCGGAGCTCAGGAGAGGGATCTGGCCACAAAGATGGGG | −0.05603577 | Hypomethylated with Age |
| | GGGG[CG]GTGCACAGAGGATTCTAAAGACACAGAGTGGCACAGAGGGCAGAGAG | | |
| | CCTGTGGAGATGA | | |
| cg00281467 | CAGGACTGCAGAACTGGCCCAGACCTCTGTATTGGAAAGGTCTTTATGGACCAGG | −0.14217206 | Hypomethylated with Age |
| | GAGTC[CG]GTGTCTTTTTTACGGGGGACCCCTGGGCTGCGAGTTGCACAGTCCA | | |
| | ATTCGCTGTTGTTA | | |
| cg00288562 | CAGGCTCCAGCAAAATGGCGCCGGCGCCGCCAGAAATCTCCTGGCCTCCTCAGAG | −0.10459208 | Hypomethylated with Age |
| | CACGA[CG]TAAAGGGGGGGGGCGTCTCTGTGACGTCACGAGGCTCCACCTCCCG | | |
| | CGAGGCTTTGTGTC | | |
| cg00292435 | TCACACCGCAGAACCTTTCTGCCTTTTACTACTTTTCAAAGTGGACCTAGGCTCC | −1.38184967 | Hypomethylated with Age |
| | TGGCT[CG]AGCCTGAGGGGATACAAGGGATCACGAGGAGCGCCATCACCGTGCA | | |
| | AGGTCGACAGCTTC | | |
| cg00316222 | CTAATGTCCAAAAGCATTCCTAATACCAAGCATAAAGAATGTTTCTTAATTCACC | −0.08225597 | Hypomethylated with Age |
| | AGACT[CG]CCAATAAGATAGTGACATACACTAAGTCTTCCTGGTTATTTAAAAA | | |
| | AGGAGGGGAGGGGG | | |
| cg00347798 | CGCGGGGGGGGGAGGAGGTCCCAGGAGCCGGTTCGAAAGCTCCCTCCGTGATGAA | 0.01941892 | Hypermethylated with Age |
| | GTAGG[CG]AGAAGGGAGGAGGTGAAGGAGGGCGAGCTGAGCACACGCGCTTCAT | | |
| | GCCACAGGAGGGTG | | |
| cg00356500 | AGAGCGAAACTTCATCTCAAAAAAAAAAAAGAAGGAAATAAATGAAATAAGCCAC | 0.04247688 | Hypermethylated with Age |
| | AAGAG[CG]ATAGAGGAGTAGGACAGATGGAGTGTAAAGAAGGAGAAGACATTAT | | |
| | ATATTTTCTCTTTT | | |
| cg00359604 | CGTGGAGAGTGGAGACAAAGATGCTCAAAAGCCAGGAAATCGCAGGCTCGGAAGC | −0.06929177 | Hypomethylated with Age |
| | CCCCA[CG]CATGCCTCTGAACGCAGCGCCATCTCGGGGCTGCGGCGGGACCAAG | | |
| | CGGGACGCTTGCAG | | |
| cg00361467 | AAGTTTGAGTGGGGATTTGCTTTCTGGAATTGACTGTCTCCTGTCTTCAGATGAC | −0.00167506 | Hypomethylated with Age |
| | TGCCC[CG]TGTCAGCCCGGCGGCAATCGTTAGTCTCCGGGCCAACCCAGGACGA | | |
| | TGCTTTTTGGCGTT | | |
| cg00395697 | CAACGCCGGCTCTGGGGGGGCTCTGGGGGGCTCTGGCTGGGCTCTGGCTGGGCTC | −0.03701335 | Hypomethylated with Age |
| | TGGG[CG]GGGGCGGGAGGTTTGTGGCATTGGCGCCAGGGTTCTTAGCCCTCGCG | | |
| | ACACAGGGCCCTT | | |
| cg00499399 | TTCCTGGCGGTCCTCGGCGGAGCGGGAGCAGTGGGACGTTTCCGGGGGTCGGGTG | −0.13373984 | Hypomethylated with Age |
| | GGTAG[CG]GCGAGCGCTGTGCGGTCAGGGGGGGGCTCCTGTGCCCTGTCGGTGG | | |
| | CGCAGGGAGCTGGA | | |
| cg00562504 | TTTGTGCTTTTTCTCATACAGAGTATCTCCCTGTCATAGAACCTACACAAAGCAG | 0.05050757 | Hypermethylated with Age |
| | TGTGC[CG]GCCCCCAAGAAGTCTGAACACCTGTGGCAGACAGCAGCGTCAAGCA | | |
| | GCTGTGGTTAAAGC | | |
| cg00583733 | TCCCTCAGGACTGGGCACCTCGCTGCCCCCGCTGCTGCCCACTCTGCGACTGTGC | −0.02974832 | Hypomethylated with Age |
| | CTGTA[CG]TGCCAGCTCCCCGACTGCCAGAGCCTCAACTGTCTCTGCTTCGAGA | | |
| | TCAAGCTCCGATGA | | |
| cg00590036 | AGGGCGGCACTGAGATTTTTGTCCTGGGCGGCAGACGACCTTGTGTTGCACTTCC | 0.59718479 | Hypermethylated with Age |
| | TCCCC[CG]CCTTCTGCCTCTCCCGGGGGGGGGGGGGATGGGCGCGGAGGCGGAT | | |
| | GGGCGCGGCTCCCT | | |
| cg00593462 | TCCTCTGCCATCATTTGATCCTCTACCCGCTAAAAAGCGGGTTTTCCTTCTGGGA | 0.43156417 | Hypermethylated with Age |
| | CTTGG[CG]CAAGCGCTCCTAGGCCAGGCGCGCGCTTAGGTCTGAGACCGGCCGA | | |
| | GGAGCAGGGGCGCC | | |
| cg00602326 | TGACCCTCTGACTAAGGATCACCGCAGATACTTAATCGCCAAGCTGCCCTTGCCT | 0.25253475 | Hypermethylated with Age |
| | TGGGA[CG]GCACCCAATCCCAAAGTAATCTCTTGCTGCTCCTAAACTGGCCACA | | |
| | GCCAAGAACCTCCT | | |
| cg00610228 | CCAGCGCGCCGGGGCTGGAACACAATGTCCCGAGGGGGGGGGGGGGCGGGCGAGC | −0.30005959 | Hypomethylated with Age |
| | GCGAG[CG]AGAACAGCCTGACTCAGCAGCTGGGTAAGTGGGTGTGCTCGCTCAC | | |
| | CAGATCAACCGCTC | | |
| cg00658405 | AGAGTGTGGCGAGAACCCCCAGGGACCCCCTCCCAGGGCTGGCCTTCCACCCTCC | 0.01333548 | Hypermethylated with Age |
| | GCCCC[CG]CCAACCCCACCCGCATTCCAGCGGGATCAGGGGGATTAAAGGGCAC | | |
| | AGCATGTGTGGGCA | | |
| cg00663832 | TTCACTGCCGGGGACCTCAGTTTGCCCATCTGTTAAAGGAGCATGTTGAACCAGA | −0.71950638 | Hypomethylated with Age |
| | GGACC[CG]CCAAGCCCCTTCCGAGTGCCTACATGTAATCCTCCCTCCTCTCTCC | | |
| | TGGACCACAGCGCC | | |
| cg00716277 | CAGGAGGGACCTTGAGGAGGAAGGGACTCCTCGGTCATACCAACTCAGGAAGTGG | −0.05677381 | Hypomethylated with Age |
| | AACTG[CG]TCACGGTGCGCGTCTCGTTGGTCTGCAGGTTCTTCAGATAGAAGCT | | |
| | CCTCACCAGGAAGT | | |
| cg00753885 | CAGCCATCTCTGGAGGGTTGACCCCAATAAACTTCACATGAAAACAAATCATCCA | −0.20000336 | Hypomethylated with Age |
| | AAAGA[CG]CAGGTGAAAGTATATACCACTTATACTGAAGTCTTTTTAAAGTAAA | | |
| | TCACCATATAGTCA | | |
| cg00798886 | TTTCTTGTTCTTGCCGCCCATGTTGCAGCTGTGGCAGAAGATCCTTCGCGGCCCA | −0.5058317 | Hypomethylated with Age |
| | GGCCC[CG]ACGGTACCACTGCACAGCCGAGAGCTCTTCACATTCCCCGGCTCCG | | |
| | GGGCTGCCACCCTG | | |
| cg01019875 | ACGGCCAGCAGCCGCGGGAGGGGCACCCAGCCTTGGTCTGCCAGCCACCCGCAGA | 0.30898533 | Hypermethylated with Age |
| | CCAGC[CG]GGCGCCCGCATCCCCCATCGCAGCCACGGCCACCACCAGCGCTGCT | | |
| | CCGTATCCCCCCAG | | |
| cg01045132 | CCAAGGGCCTGACATCACAAGGGGAGGGGAAGGCAGCTGAGGTTGTGGGGGGAGG | 0.01068176 | Hypermethylated with Age |
| | TGCCC[CG]CCCCTTGGCAGGCCCCTACAGCCAATGGAACGGCCCTGGAAGAGAC | | |
| | CCGGGTCGCCTCCG | | |
| cg01078197 | CACGTAGCGCCGGCTGCGCGACAGGGCGCGCGCGACGCTGCCGACGGCGCATGCG | 0.03598188 | Hypermethylated with Age |
| | CGGTC[CG]GCATGCCGGGCGGAAGCCCCATTTGATTTCTAATGCTATTTATTTA | | |
| | TGTATCCCTTGTCT | | |
| cg01224366 | CTGGGCAGGGGATGAGCTTGTGTCGCGGGGGGCAGGGGGAAGGGAGTCGGAGAGC | −0.37706777 | Hypomethylated with Age |
| | TCCTG[CG]GTCCAGCCGGATGACTGATGAGGTTGAAAGCACTTCCGCTGCGGCC | | |
| | CCCGCAGGAAGTTC | | |
| cg01265531 | TAAAGGAGGGGGGACGCCAGCAAAGCGGGGGCACAGGGGAGGGGCGCACGCACAC | −0.29341352 | Hypomethylated with Age |
| | GCACA[CG]CACACATGCCCGCACTCACAGATGGAGATTCTGTATTGGCAACTTA | | |
| | TCCGGAATCTCAAC | | |
| cg01381617 | GGGTGTCTCTCCCTGTTTTACGGTTGAGGGCCTTGGTCAAGCCCTGCAGCCTTGA | 0.00020033 | Hypermethylated with Age |
| | GGTTC[CG]TTCCTCATCCAGGAAGCCCGCGAGGCACGCTGCAGGCAGGTCCACC | | |
| | GGCCCGCGCATCCC | | |
| cg01437235 | TGCCTGGAATGGACCCTTGGAGGGTATGGCACTACACCTTATTAACTGTTGCTAA | 0.03904157 | Hypermethylated with Age |
| | ACTGT[CG]CTGAAACACACATGTCCGTTTACATTCCTGCCTCAGTTTCTGTTTC | | |
| | CCCAGGCCCCCGCC | | |
| cg01447660 | TCATCACCTTGTGGCCAGACAGGATATTGCTGTTAGAGACTCCAAGAGCCTGTTT | −0.35506842 | Hypomethylated with Age |
| | GGGTT[CG]GAGCTATTCTGGTCAATTTTATCACCCCATGCACTGCCTCCACTTA | | |
| | CTCATGGGCCAGGG | | |
| cg01474003 | CTCAAAGGTAATTTTCAGCTGTGCCTCATGAGGGGCTGGTGTAGGAACTGAGAAC | 0.13350705 | Hypermethylated with Age |
| | CACCA[CG]ATGGTGTCCTGGCTTCTCTGTCTGGGTCTGCTCATGTCACGTCTAT | | |
| | TCTCTATGGCTTTT | | |
| cg01630444 | CTCGGGTGATCCAGCCACCTCGGGTTCCCGAAGTGCTAAGATTACAGGCATGAGC | −0.027546 | Hypomethylated with Age |
| | CACGG[CG]CCTGGCCAGGATTTTTAAGGTTGAAGCATCCACAACAATTTTGTGT | | |
| | GTGAAATGAAATGA | | |
| cg01635063 | CGTCTGCCCTGGCCAGGGAAGGTGCCCAGAGGGGGGAGGCCGGCCGGATCACACG | −0.11475203 | Hypomethylated with Age |
| | GACCT[CG]CAGGCCCTTCCCAGACGCTGGGTTCTGACCCCGTGGGGGCCCCTCC | | |
| | CTGCCCAGTTCTGC | | |
| cg01689404 | TTCCTTTTAGGGTGAGCCTTTGGTTCTCCTGTCCCAAACCGAGGGGGGCCACGTG | −0.00710458 | Hypomethylated with Age |
| | GGAGC[CG]GCAGCACACACTGGCCACCACTGACCGCTCCTGCTGTCCTGGCACT | | |
| | TCCGCCCCCTCCCA | | |
| cg01747664 | AACGTTCCTTTCTGGCACGACCAGTATCTGAACATCTCTCACAATACCCACGCCA | −0.06195407 | Hypomethylated with Age |
| | CAGTA[CG]AGACACTGTAGCCTAAGAGTAAAGCCTAACAGTCAGCTCCTAGCAT | | |
| | TAGCTTTGGAGTTA | | |
| cg01793416 | AAACTTTTATTTACACTGGGTGGCACACCCCTGAGGCCCACACCCATCACCCAGG | −0.06956824 | Hypomethylated with Age |
| | AGTAC[CG]CAATCTTCCTGATCCCCTGGACTCACCTGGGGGGGGTGCAGACTGC | | |
| | GAAGGCGATAGTAC | | |
| cg01845244 | GCGAGGGACCGAGTCTGGGAGAGCCTTCCGAGTCCGGCCTCCTGCCTTCCCGCCA | −0.08706108 | Hypomethylated with Age |
| | TGGGA[CG]GCTCGCCCCAATCCCGCGAGGCTCCTGCGCCTCCGCAACCCAGGCC | | |
| | CGCACCCCTTTCCC | | |
| cg01866597 | CCGGTGCGAAGCCACCGAGCCGGGTCCGCAGCCCTACTGTGCACCTGCATCCTCA | −0.05317982 | Hypomethylated with Age |
| | GTCCC[CG]AGATCAGTCCCCAGGGCGTCAGCTGGGGAGTCAAGGAGTCAGCCTG | | |
| | CTCCGGGCTGACCT | | |
| cg01894064 | AGGCATTCATGGACATTCTTCTTAGAAGGCCTAGTGCTAAGTCGCAGTTGTCAAG | 0.39658351 | Hypermethylated with Age |
| | GGAAC[CG]CGTGTCTGGAGGGAGAACAGGCTCTCCGGAGTTTCCCGGGAAACCA | | |
| | CCCCCCGCAGAGGC | | |
| cg01967399 | TACACTTCACAAACGCACTAGTGCCCACTGTCTTTATCTGGAAGGGGTTGAGTAG | 0.01541148 | Hypermethylated with Age |
| | GTTGG[CG]GGGGGCAACGCTGGTGTCTACACAGCCAAGAGGGAACATTCACGCA | | |
| | CACAGAATCTATGA | | |
| cg02071825 | GCCATGGCGACAGTCTCTAGGCAGCGTGGGCTGAGCTCTCCGTAGGAATAAAGGG | 1.6232439 | Hypermethylated with Age |
| | TGGGC[CG]CCGCTGGACCCTGCGCGCGCCCGCCACCAACTTTCCCTCCAGATCC | | |
| | GAGAGGGGCGGCGC | | |
| cg02116471 | TGGCTCGGCAGCCCCCAGCCCCGCCCTGCGGCCAGGCACACATGCGGGCACAGGC | −0.16832793 | Hypomethylated with Age |
| | AGGGG[CG]CCAGAAACTCAACTAGAGGACACAGCAGCTTCAGGAACACTGGTGA | | |
| | ATTCCGCCGGACTT | | |
| cg02121104 | ACTCCATGGACGCAGCGACGAGAGTGATCAGCAGGAGTCCCTGCACAAACTGTTG | 0.01682036 | Hypermethylated with Age |
| | ACATC[CG]GAGGCCTAAACGAGGATTTCAGCTTCCATTATGCCCAACTCCAGTC | | |
| | CAACATCATTGAGG | | |
| cg02122920 | GTCAAGCCCCTGTCAAGCCACCTGGTGCTCCGTGTCCTGTGGATGGTAGCTGCCC | 0.48122773 | Hypermethylated with Age |
| | TGAAG[CG]GGACTTTGCAGACTGAAGTGCTGTCTCTTCAGAGGGAGTGCAGGTG | | |
| | TCCGGCTCCTGGTG | | |
| cg02136132 | TCCCTAAGCCCCGGCAGCCGATTCGGAGACTCGGGAGGCCACAGGCTCAGCGCGA | 0.41689152 | Hypermethylated with Age |
| | CACCA[CG]ACCACAACTAGGAGGCACCATCGTCGATCTACCTGGGGAGGCACCT | | |
| | ACAAAGCCAGCAGA | | |
| cg02174884 | CTCCTCGATGATGGCGTCCAGCTCCTCCTTGGTGGGTGTCTGGCCCAGCATCCTC | −0.02001434 | Hypomethylated with Age |
| | ATCAC[CG]TGCCCAACTCCTTGACGCTGATGTCCCCACCACCATCAGCATCAAA | | |
| | CATGTCAAAGGCAG | | |
| cg02189001 | AGAGCTCAAACTCCATCCCTTGGCTCAGGTTTGCCCCTTTCATCCTTCTCCAGTG | 0.00090906 | Hypermethylated with Age |
| | CTCTG[CG]ATGGAGAAAGAAGGCACCACCATAAATGAGGTTGAACACCTGGGGC | | |
| | TCTGACTGGGTTTA | | |
| cg02219949 | GGTGGCACTGATAGCCTGGGAGAGGGAGGGAGAGGAGGACGGGGGCACCTACTCA | 0.00024354 | Hypermethylated with Age |
| | GGCTC[CG]GCTGCCTCACCACAAGAGGAAAACAGGGTGAAGTTGCAGGAAAGGA | | |
| | GCCCCAGGCCGGGG | | |
| cg02278912 | GGGCCAGCAGCTGCTGTGCTGGATTTCTGGGGTCTTAGCTGTCCCAGCGGGGCAG | 0.05517736 | Hypermethylated with Age |
| | GGTTC[CG]GACCTGCACCCGGCCATGGCCATGCCTGAGCCTCCCCCTGCTCCCC | | |
| | CCGCTCCCCCCACC | | |
| cg02299189 | CCAGAGAGCACATCTTGCCGGTTCGCAGGACGTCTGCAGTCGGCAAACTCCTGGC | −0.05236946 | Hypomethylated with Age |
| | CGGAA[CG]GCACAGACCGCACTCCCGCAACTCGGTTCCCGGGCTAGATTCGTAT | | |
| | GCGGACGGGTACCG | | |
| cg02351381 | GGCACCCGTCTCCACCTCCCCAGCAGCTGTTCCCTGTCATGTCGGTGCATTTACA | 0.02289156 | Hypermethylated with Age |
| | ATGAG[CG]CCAGTCGCCTGTCTCCAGGTGGTCAGAGGTTGAAATCCCTTTTGAA | | |
| | AAGTTCTTTAAAAA | | |
| cg02394686 | CCGTCCGTTTTCCGCCCACTTGGGCCCAGCCGTCCAATCGACACTCATCATGCTC | 0.07064516 | Hypermethylated with Age |
| | TGCCT[CG]CCGCTCTCTCCGGCCAATCCGCATGTGCCACTGCCTCTGCCCGCAA | | |
| | TCGGCGCTCACCAA | | |
| cg02401639 | CAGGATCGGCCGGGAGGCGAGAGGGATTTTGTTGAGGAGCAAGGTCTTCCACAGG | 0.09526018 | Hypermethylated with Age |
| | AACTG[CG]ACTTGGAAAGTATTCACCAAGGGCTGTGCCATGCGAAACCCTCTTT | | |
| | AAAGGAACCGCATC | | |
| cg02605776 | ATTTACTCCTCTTTCCTGAAGCTTTGCCTCTAGACTTACTTAACTCTTTCCTCCA | 0.05355985 | Hypermethylated with Age |
| | GCCTA[CG]TTCATCGAAACTATCACTTATTGTCATATAGACCTTTATTTCTGAA | | |
| | GGGAGATCTGGGAG | | |
| cg02618733 | CAGGGTCACCACCTGGTGGTACTTCTTGACCAGGGCCACGATCAGGGCCTTCTGC | −0.05441225 | Hypomethylated with Age |
| | TTGGG[CG]TCACGCGGCAGCAGATGACCGCCTGGCACTTGGACGCCAGGTCCAC | | |
| | GAAGGCGCGCTCCT | | |
| cg02698770 | CCACACCTCTCCCCTGCCCAGTATCTCCCCCATTCTACCCCAGCCCATGGCCTTG | −0.04685093 | Hypomethylated with Age |
| | CGCCA[CG]CTGCTTCATCCATCCTGATGCCCAGTATGTAAAAGGCGCCTAACCC | | |
| | GCCATCATGTCCCT | | |
| cg02705835 | CCACAAAGTCTGGGGGGGGAGCAGATTGGGTACCAAGGAGGACTGCCTGGAGGTG | 0.00271012 | Hypermethylated with Age |
| | GTGGC[CG]CGGTGACTGCTCCAAACCCGTTCGGCCCTGTGCAGAAGTCTGAAGA | | |
| | ACAGAGGTGCCCTC | | |
| cg02741548 | CTCCTAGGATACAGGCTGGGCAAGGCAGATAAGTGGCTCTTGGCCTGGTGACCTT | −0.02557409 | Hypomethylated with Age |
| | TCCAG[CG]TCCAGTTCTTTGGAGTAACCACTTCGCAGAGCCTCATCCAGCCGGA | | |
| | GGAGCCCCAGGCTA | | |
| cg02821342 | CTATATTAGGGCTTTGTTGCTGACAACAGTGAAAACTTGTTTGTGTCAGGAAGTG | −0.96155196 | Hypomethylated with Age |
| | AGGTA[CG]GAGATATGACCTGGAAGGTACAGACAAAACCAAAGTGGCAGTTTTT | | |
| | GCATTACTTTTCTG | | |
| cg02835038 | CCAGCCTGGCAGGGGATTTTAAAATCGGCCAATCACAGCGGGGGCCAGGCCTCCG | −0.04124323 | Hypomethylated with Age |
| | CTTTC[CG]CTTACTGGTCCTGCCGTAGGAGGGGGTACGTGAGCGCACCAATCTG | | |
| | TGGCCGGCGAGCG | | |
| cg02835848 | AGAGAGACACAGCAAAGTGGGGTGCCAGGCAGAGGCCAGGGGCTTTCAAAGACCG | −0.01383153 | Hypomethylated with Age |
| | GGGCT[CG]AGTTCCGTCACAGCCACCCCTGTGACATCGGCCGTCTTTCTGACCC | | |
| | TCCGTGTCCCCGGG | | |
| cg02962380 | TAGCACTCGTGACGTCAGGTCCAAATGAGAAATTGACTGGCACTCCGGGCCAATG | 0.00707469 | Hypermethylated with Age |
| | GGAGG[CG]GCGAGAGCGGCCGCGATTAGCATAATAGTATAGAAACAAGGAAACT | | |
| | TTTCGGAGCTGTCA | | |
| cg02969038 | TTCTCTGATCCGTAAGAGCCTCCTGCCCGTTCATCCATCAACAGCCTCTGGGGAC | 0.00925889 | Hypermethylated with Age |
| | ACCTT[CG]GCAGCAGGAATCACTGGTGTTGGGTACTGAGTTTGAATTTCTGTGC | | |
| | CCACCTTTGCGCTG | | |
| cg02976543 | TTGGTGGCCGCGCCCGTCGCTCTTTTTACATAAGACGCACATGGAACTCCATGTT | −0.01820926 | Hypomethylated with Age |
| | CACCT[CG]TCGGTTCCTCAATGGAGACGCGGCGCGTTCGTGCTACCCGTCGTCC | | |
| | TCCCTAGTGGTCTC | | |
| cg03004599 | ACACGCCCGTTCCGCGTCCGTCTCTGGCTCCGCACTTGCTGCCCTCTCGCCGCTC | 0.02243404 | Hypermethylated with Age |
| | ACATT[CG]CAAAGGGGGACAGACACTCATCGGATAATGACACAGCTGGACGCAG | | |
| | AGCCCCGGAGAGTG | | |
| cg03052071 | GATTGGTGAACGTCGCCTGGCGGTGTGTATGCTTTGGAATTCGTGGTTTCCTCTG | 0.0275587 | Hypermethylated with Age |
| | GGCCT[CG]ATACGCCATCCATTTTTGTGATTGTTCCATGGGCACTTGAAAGGCC | | |
| | TTCGATGAATATTG | | |
| cg03068319 | AACAAACCCTCCGGCCTGGATCCCAAAACAACAGTCGCGGTTCTGCAACAGAAAA | −0.06606652 | Hypomethylated with Age |
| | GGCTG[CG]CTGGCCCTGGGACCTGTCTCGGAAATACTCCTCATCCATCTAGTTT | | |
| | CTCCCAGGACAACT | | |
| cg03121508 | ATCAGACTTAGGTCACAGAATTCAATGGTTTCTGACTATTTTATTTAAACTGGAA | 0.00828873 | Hypermethylated with Age |
| | ATCGG[CG]GGATGGCAAGGAATACTACTTGCTTCTATAGTGTGTGATCCACATT | | |
| | AGTGATTTGTGGAA | | |
| cg03138206 | CCACACACCCTTAAGGTTTTTCACAGCACTCTGACGGTATTATGTGTGTTTTGCA | 0.05324147 | Hypermethylated with Age |
| | AATGA[CG]AATCAACAGTATGCTGAATAATCAGCAATGAAACACAGGAGATAAA | | |
| | TTAAATGTGTTTTT | | |
| cg03176453 | GAAACAGAATCCCCGCGTGCCCCTTCCTCACTACCCTCCAAATCCCGCTGCAGCC | −0.41561331 | Hypomethylated with Age |
| | ATTGC[CG]CAGACACGATGCCGAAACGAAAGAAGCAGAATCATCACCAGCCACC | | |
| | GACACAGCAGCAGC | | |
| cg03244036 | AGTGGACAGTGCCGTACAGTAATGTCTACGGGGAGTTCCAGGAGAGCTCGGCTAC | −0.43173598 | Hypomethylated with Age |
| | TCCTG[CG]CAGGATAACCTCTCCCCCACCACCCGAGTCCCGTGCTCGCGGGCAG | | |
| | GACTTTTCCGAACT | | |
| cg03277925 | GAAGGGGGTTCTTACCGCTGAGGAGAGGCCGGTTGTGCCGGTAGGAGGCGGGCAG | −0.14911794 | Hypomethylated with Age |
| | CTGGC[CG]ACACCCTCCATGCGGTGGCTCATGACGCGTGCGAGGTGGCCCGTGT | | |
| | GGTGCAGGCTGCCC | | |
| cg03314029 | GTTGAAGCTGTTCCGGAAGGATCTGGACTCGGGCGGCGCAGCAGAGGGGTCGGGG | −0.00216868 | Hypomethylated with Age |
| | TCGGG[CG]GCGGCGGCAGAGCCTCCGGCCTGAGGCCCCGGAGGAACGACGGTCT | | |
| | CGGGGAGCGGCCCC | | |
| cg03382370 | CTCCGCGCTCAGCGGGAGGAGGCGCTCGGTCCCGCTTCTTACAACCAGCGGCGCT | −0.0533514 | Hypomethylated with Age |
| | CACGG[CG]GGCCCGGGGATCAGCATCCCGGGAGCTTCTCAGGAATGCAGATTCC | | |
| | CAGGCCCTCACTGC | | |
| cg03404662 | TCCTTCCTCTTGTCGGGGCCTGGGCTTAGGACACGCCCGTTCCGCGTCCGTCTCT | 0.089359 | Hypermethylated with Age |
| | GGCTC[CG]CACTTGCTGCCCTCTCGCCGCTCACATTCGCAAAGGGGGACAGACA | | |
| | CTCATCGGATAATG | | |
| cg03539970 | AGTATGGGAGTTTTTAAGAAAGCTTCTAAAAGGCTGAAAAGATACGCAAATGAAG | 0.15132123 | Hypermethylated with Age |
| | AAACT[CG]GGGCTCAACACACCACTTTAAAGGACTACAGGATCCTGTTTGCAGA | | |
| | GTTTAGAATGAAGA | | |
| cg03595220 | CGCAGCCGCTGGCCTGGGTGCTAAGCCCCTCATTGCCCGCGGCGGCAGGGCTGGC | −0.05255514 | Hypomethylated with Age |
| | AGGGC[CG]GCTGCGCTGCCGGCTGTTCCGAGTGTGGAGCCCACCAAGCCCATGC | | |
| | CTACCTGGAACTCC | | |
| cg03651054 | CCCACTGTGGCTGCACCCCGAGGAGGAGTCCGTGGCAGGAGGCCCACACCCAGGA | −0.00433085 | Hypomethylated with Age |
| | CCCGG[CG]CCAACAGTGAGGAGACGAACCTGGAAGACATCCCGCTGTCCAGACA | | |
| | CCGCGAGGGAGGCG | | |
| cg03772253 | GTCGGCACACAGAGGTCGCTGCAGAAGCCTAGCTGTGGCTGTTCTTAGAGCAGCA | 0.05093277 | Hypermethylated with Age |
| | AGCAG[CG]TTCTCGCCCTCATCCTGATTACTAAGGCATGAATAGTGCTGCTTCT | | |
| | GATGGCCGTTTCCG | | |
| cg03786043 | CGGTAGGAAGACTTGAAAGGGGTGGTCGAGGCACGTTTGTGGTTTGAAAGGAAAA | −0.0430369 | Hypomethylated with Age |
| | AAACA[CG]GAGCAGCAGATGTCCCCCCCAAGGGTGAAAGTGCCTCCCAATCGCA | | |
| | CATGCGAGGGTCTC | | |
| cg03831869 | GTGGTCGGGACAATTTGCAACTAGAGGGTGGTCCTCATGGGTACCCTGTGGGGTG | −0.46278595 | Hypomethylated with Age |
| | TCGCG[CG]AGGTGAAGGGCCAGGCCATTTCCGTCGGGTCAGCGATTTCCGCCTT | | |
| | CGCCCCGCTCTCGC | | |
| cg03906843 | TGGGTGACAAAAATGAAACTGTTTTAGAAAAAAATAAATTTTACTTTGTTAGAAT | −0.06565584 | Hypomethylated with Age |
| | ACCAA[CG]CAAGGTTCCTTAGAAGGTTGTTGCAGCATCTGCCCTCTATGGGCAG | | |
| | CTGTACAGTGACTA | | |
| cg03915012 | CTGGGCAGAGGGTGCGGCAAGGCTCACACAGCAGGCAGGCTGCAGCTCCAGGAGA | −0.10474541 | Hypomethylated with Age |
| | AAGCC[CG]CAGGCCTCAAACAGAAGACGCAGTGATGCATCCACACCAGGAAAAC | | |
| | CAAGCAGCCTTCTG | | |
| cg04026169 | GTCAGCATGAATAGTGCTAAGGAGTCAAGCCAAGACTTGCTTCAGAAGAGAGCTG | −0.11721579 | Hypomethylated with Age |
| | ATGTG[CG]GACCTTCTGCATTTCCACGCGGGGATCCAGTGCTTGGGTCTAAACC | | |
| | CCAGCGCCCTGCCA | | |
| cg04198125 | AGAGGGAGCCCGGCCCCTGGGTGTGCTCGGCAGCTGCTGAGATGATGGCAAGGGG | −0.0302221 | Hypomethylated with Age |
| | GAGCG[CG]GCACCTGTCGTCGTCACCTTTGTGACGTCCCAAGCACTGGGGGGCC | | |
| | CTGCGGGGGGTGGG | | |
| cg04271792 | TGAAGAATTGAGAGAGAATGAAAGTGCCACAAAAACAAAAGAAAAAAAATTGAGG | 0.29589891 | Hypermethylated with Age |
| | CGAGT[CG]TGGACATGATAGACATGATTTTGCAAACAAGGCACATCTAGGAGAA | | |
| | AAGGCGGGAGAAAA | | |
| cg04403868 | GTTGGGGACGAACTCTTTGCTAAATAAACATTTAGGTGAAACAAAGCTTCCATTT | 0.10460297 | Hypermethylated with Age |
| | CCAAT[CG]GCCTAACTCTGGTATTAGCGTTGGCAGGGGCCATGCAGGAGGGGCA | | |
| | GTGAGCAGTGGTGG | | |
| cg04406111 | GGCTGGGAAACGCTGATTCCCTGTGGGCAGGAACGCTTGTCTAATTGCGACGGCC | 0.02674701 | Hypermethylated with Age |
| | CTGCA[CG]GCTCCTGCCCAGGTATGAAGTGCAGCTGGCTTGCACCAGTGCGAGG | | |
| | GCGGAGACGCGCTG | | |
| cg04420141 | CCGTATTTTCCACCGCGCTGTATTAGTGAGGGCTCTTTGTGTCACTTCTGTGCAT | −0.25915998 | Hypomethylated with Age |
| | AACTC[CG]CCCCAAGTTAAAAGGCTCGCTGCTCTCGACAGGTCCTCCTCCTCCT | | |
| | CCCGCTAGGTCCTC | | |
| cg04434896 | AAACCCCGTTTTACCCCAAGGGTGGATAGAAGGGAAATGCTGAGTTTTCATGGGA | −0.01945834 | Hypomethylated with Age |
| | TCTCA[CG]CCAGGAGAAAATCAGGAGCATGGAAGGGGTGCAAGTTCATGGCAAG | | |
| | ACGGGACAGGACTC | | |
| cg04501188 | GCGGCGGCCCGCGGCGCAGCGGGGCCCGGGCCGGGACCGCCGTCGGGGGGCGCGG | 1.66896274 | Hypermethylated with Age |
| | CGACG[CG]GAGCCCGCTGGTGAAGCCGCCCTACTCGTACATCGCGCTCATCACC | | |
| | ATGGCCATCCTGCA | | |
| cg04514392 | TGGGCCTCAAGCACTTCACAACATGATTATTTTATTCCTAACGGTCAGTAGCGAA | −0.08493077 | Hypomethylated with Age |
| | TAAGA[CG]TAAAATGACATAGTTCTGCTGTGGGTAAACTCAAGGTTTTAAAAAG | | |
| | GAAAAACTTCATTG | | |
| cg04561005 | GCCCAAACGCTGGCTCAGGCTAAGGCCAGGTGGGCTAATCTCAATGTTGGCCTGT | 0.03079815 | Hypermethylated with Age |
| | GGTGG[CG]AAAGTCTTGTGCAAATGGCATATGTTAGGAATCACTCCCAGAGGCA | | |
| | GGACCCTTGAAGGC | | |
| cg04578193 | CGGTCTCTTTTCGGGGTGGGAGGTCTCCCACTGGGACCGACACAGACGCACTGGA | −0.5271438 | Hypomethylated with Age |
| | TGCCG[CG]GGGTCCCGGGCTCCGAGCGGCGGTGTCCCTGTCCCCTTACTCCTAC | | |
| | CCACCCCCACCCAC | | |
| cg04590721 | TGCAGGTCAGCAAACATTTCGGAGCACCCAATTTGTGTCTGGTCCTCGCTTATTG | −0.11717919 | Hypomethylated with Age |
| | CAGGA[CG]ACCCGGGTTATCGGACACCCCCCCTTCCCACCCCAATCCCACCCAA | | |
| | CTCCTGTCACCTTA | | |
| cg04596060 | ACTTGGAATGAACATGTTGGAAATAAACGCTCTCATTTTGCAGGCAGATAAACTG | −0.4549745 | Hypomethylated with Age |
| | GGAAT[CG]TGCGTGTAAAGCAGCTTGCTCAAAGTCTTATAACTATGAATTGGAA | | |
| | AGTCAGATTCGAGC | | |
| cg04606053 | GAGAAGAAACCGAGGATGTTCAAGCTGGAGAAGGGGGAGGGGGAGGGCGGAGGAA | 0.12390797 | Hypermethylated with Age |
| | GGACT[CG]ATCACTGTCTTCGTAGCTGAAGGGCTGTCAGGTGGAGGTAGGATAG | | |
| | ACTTGTTCTAGCTT | | |
| cg04622620 | GAAGGAGCCGGAACCGGAGCGGGCAGGACCTGAGGCTTCCCTCGCCGGGGCAACG | −0.05732559 | Hypomethylated with Age |
| | GCTGC[CG]CCGCAACCCGGGTCCCACCAGCGCCGCTCCACCTGCAACGGTCCCT | | |
| | CAGGCTTTAGGAGA | | |
| cg04673462 | CCGCCTCCACCCTTGACTTGAATCACTGTTGGCGGGGGACGGGGCGTGACCCATT | 0.00041485 | Hypermethylated with Age |
| | CATGC[CG]GGAATCGGATCCAGATGTTCCCGCGGCGTGTGCAGCTGCATCCTTG | | |
| | CCTTTTTTGGCAAA | | |
| cg04677061 | GCCCCTCTGCTCCGGCTCGGGGGGGGCACTGGCGGAGGGACTGGCCAGTCCCCTC | −0.21121454 | Hypomethylated with Age |
| | CTCCG[CG]CCGGCCCCAACCCTGTCGCTGCCGCCGCGCTCCGAGTCCCCATTCC | | |
| | CGAGCTGCCGCTGT | | |
| cg04732357 | TGCTGGTGGCGGCTGCAGCTGCAGCGCCCGTGGGCTGACGTGGCTTCCCGGAGCT | −0.08995077 | Hypomethylated with Age |
| | GCGGC[CG]GCCAGCGCCCAAGGGCCCACAGGCTGCGCTGCCCTTGCCAGCTGCT | | |
| | TCTGACCCGCGCCC | | |
| cg04777612 | TGCACGCAGCATCGGCCCTACACCTGCTTCCACTGGCACTTCGTGAACCAGCGGC | −0.23713362 | Hypomethylated with Age |
| | GCCGC[CG]GTCCATCCGCCGTCGGGACGGCACCTTCAATTACAGCCCTGACGTC | | |
| | TACTGCACCAAGTA | | |
| cg04821107 | GAGCGGCCCCGCGGAGGAGCCACCGGAGGCTGTAGTTGCCGGGGAGTCCCGCATT | −0.04395447 | Hypomethylated with Age |
| | CAGTC[CG]CTCAGCCTCTGGGCCGGGCCTCGGCGGCCCCCAGAGCCCCACACCC | | |
| | GCAGGCCCAGGGCC | | |
| cg04836038 | CTCTGCGGGGACAGAGGTCTCAGGAAAGTAGCCTTTATTTATGTGGCACCGATCG | 0.48840761 | Hypermethylated with Age |
| | GAACC[CG]CGGCCGGCCAGGGGGACCTGGACGGAGCGTCCCTGCTCGGAACCTG | | |
| | GCGCGGGGCGCCGC | | |
| cg04854451 | GGAGATCTTTTCAGACCAATAACCTTCCCTGCCTCCAAAACAAAATGGGGAGGTA | 0.00083781 | Hypermethylated with Age |
| | GAGGG[CG]CTAGCGATGGAACAGATGTTCCTGCACGTCTTACGGATGGTCCAGG | | |
| | GTGGGTTTTGTGCT | | |
| cg04875128 | CGGCGCGCGCCGGGCTGTAGCTCTGCGACGACAGCGAGCGGTTCTGCTGCGGGTA | 0.59571807 | Hypermethylated with Age |
| | CGTGG[CG]CACGGCCGCAGCGCCCCCACGGCCGGCGCGCACGCCTCGTCCCGCG | | |
| | CGCCCGACGCCTGC | | |
| cg04969937 | GGGACGCCGAGCGGAGCTCTCGGAGCTCTCGGGGCTCTAGGGGCCTGGGGCTAGC | −0.01501863 | Hypomethylated with Age |
| | TGCTC[CG]CGGCGCGGGGAGCTCCGGGGGTCCAAGGAGGAGCCGCCGCCGCCGC | | |
| | CGCCGTGACGCTGG | | |
| cg04980928 | TCTGCAGAATCTGGACCGGCTGACATTTGAACCCCTAGCAAACCTGCAGCTGCTG | 0.09518696 | Hypermethylated with Age |
| | CAGGT[CG]GGGATAACCCCTGGGAGTGTGACTGTAACCTGCGTGAGTTCAAACA | | |
| | CTGGATGGAGTGGT | | |
| cg05086282 | CCTGGGTGGCGGCGGCGGTGAGGGCTGCGAGCAAGGGTGCTGCGTTTGCATTCGG | −0.3712994 | Hypomethylated with Age |
| | GGGGG[CG]GGTATGTTTCTGCTTCAGGTGGAGCTCTGTAGCGTTTCATTATCAC | | |
| | CCCAGAAATCCTCA | | |
| cg05127553 | GCACCGAGCGCCCACGGCTCCCTACGGGAGCTGGGCCCCCCGGGCCTCCAGGTTT | −0.00019611 | Hypomethylated with Age |
| | CGGCC[CG]CCCCCTGGCAGGCAGCACAGGTGGCTGAGCACCGCTACAGCGGCCT | | |
| | CTCACCGGCCGCTT | | |
| cg05331143 | CTGGCGGTAGAAGCGGCACTGCATACCAACACAAGACGTTATTTTAAGCGCGTGT | −0.01042594 | Hypomethylated with Age |
| | CCCCA[CG]AGAGAACCCATCCGATCTACTGGAGCAAGCATCTCCCACCCGCCGG | | |
| | GAATTTTCCAAAGC | | |
| cg05492839 | GTATTGGGACTCGCTGGCGTAGGGATGCTGCGCTCAAGGGTGCGACGCCAACTGG | 0.07712072 | Hypermethylated with Age |
| | GCTCG[CG]CAGGCGCGCGCCGTCGAGCGGGAGCGGGACACCTGGGCTCCTCCTT | | |
| | GGCCCCTCCCCGCA | | |
| cg05502376 | TATACTTTAAGTTCTAGGGTACGAGGCCAGGGAGAAGGAGAAGCCACCCTGAGGA | 0.03917227 | Hypermethylated with Age |
| | AGGTG[CG]GAATGTCGCGTGGAGCCCGGCTCTCTGCCTTTGAAGCAGGATTTTC | | |
| | ATGCACTCGCCAGC | | |
| cg05542681 | CCTCGCGCTACTCAATGACGAGGCAGCGGGGCAGGTGCTGCGAGAAATACTTGAA | 0.04613827 | Hypermethylated with Age |
| | GAGCT[CG]GGGGTGGCCCCGGGGCAGTTGGTCAGCTCCAGCTCCTCCAGCTCCT | | |
| | GCAGCTGCACCAGG | | |
| cg05651960 | ACCGGAGCCCGCGGGGGGGGCAGAGACCCGCCCCGGCCCGCAGGACACCCCCTCG | 0.65224375 | Hypermethylated with Age |
| | GAACG[CG]CGGCCCCCCGGCTAAGTCATGTTTAACAGCCTCAGAAATTATCTTG | | |
| | TCTCCGCGTTCTTT | | |
| cg05675373 | AAGGAGGAGATGGCCAAGGGCGAGGGGTCGGAGAAGATCATCATCAACGTGGGCG | 0.04182253 | Hypermethylated with Age |
| | GCACG[CG]ACATGAGACCTACCGCAGCACCCTGCGCACCCTACCGGGAACCCGC | | |
| | CTCGCCTGGCTGGC | | |
| cg05805236 | TCAGCCATGAGGGCATCACGGCAGCCCTGAGGCCTGTGCGGGTGCCCGGCTATGC | −0.0096839 | Hypomethylated with Age |
| | CGACT[CG]GATCCCACCTTCTCGCTGAGTGTGGATGAGGACTATGACCTCCGCC | | |
| | TGTCTGGCCTCTCG | | |
| cg05852786 | GGGCTGCGTCCCTTTTCAGCAGCTGTGTTCGGAGCTGAGCAGCTTGGAGCCACAC | 0.03453253 | Hypermethylated with Age |
| | ACGTG[CG]TTATCTCAGGGTTCCGGGACACGCGTGCTGGGGGCGCTGCCCGGGC | | |
| | CCTCACTAGGGGGG | | |
| cg05867154 | CACGTCCCAGGTGTGTGTCCGACCGTGTGTGTGAGTGTGAGAGAAGAAATAAAAA | −0.13848121 | Hypomethylated with Age |
| | GCCCC[CG]TCTCCCAAAAGCCCTGGCAAACCAGCCCAGCTGGAAAATCCTAAAT | | |
| | GCAGGCTCATCAGA | | |
| cg05915866 | GAAAAAAAGTAATTTTTTAAAAAACATAATAAATCCTCAATTGTCCCCACTGAAA | −0.22399165 | Hypomethylated with Age |
| | GGGCT[CG]AGTCTTTTTTAAATTCCAAAGTGAATTTATGTTCTGTAATTTGCAT | | |
| | TACAGCCAAGCGAT | | |
| cg05973772 | ATTGAGGTACCATATGCCCAGTTTCTTCCTAGAATCTATTCTCACTGCGTTCACC | −0.03611115 | Hypomethylated with Age |
| | GTTTG[CG]TCCCAGCACTCCCTTCGTCTGATTTGTAGCACTCCCTGTACCTGCC | | |
| | CCGGCTGCGTTGCA | | |
| cgO5973840 | CAGTATCACTATTGGCATTCCTGAGCCACTGGCTCAGAATTTCAGTACATTATCT | −0.08876636 | Hypomethylated with Age |
| | GCCCG[CG]GGACACACCTCAGAGGAAAGGGGATGAAGCGTGGTCCATGACCATG | | |
| | GCACCCCCTGGTCT | | |
| cg05994982 | TAGAGCAGCAGCTTTGTTTCTTTGCTTCACTGCTGTCTCCCTAGCACTTAGAACA | 0.07140838 | Hypermethylated with Age |
| | TAGTA[CG]TGCTCAATAAATGAATGAGTGCCTGACTGAATGAATGAGGATACAT | | |
| | AGATGAGATTCCTC | | |
| cg06002800 | CGACCCGATCTTTCTGCCCTTGATTCAAAACAATCTGAGGTCCCTAGGCCCTTCC | −0.2512952 | Hypomethylated with Age |
| | CTTTC[CG]CCTCTGCGCTCCCCATGGGGTCCGGTGTAGTTTTCCCGCCCCTTCC | | |
| | CTGCAGCTCCCGAG | | |
| cg06030274 | AATTCTCCTATGGTTCTGGAGCTCAGAAGTCTGAATGGTCTTTCGAGGCTAACAT | 0.00509476 | Hypermethylated with Age |
| | CACTG[CG]CTGTCAGAGCTGTGTTTCTTTGGGAGGCTCTTGGAGAGAACCTGTC | | |
| | TCCTCCTCACCTTT | | |
| cg06121469 | CCAGTCCCACTCTGCTTAACTGCTCTGGCATGCTTGAAGGCCTAGCTTAGCGTAG | 0.40200747 | Hypermethylated with Age |
| | CAGGC[CG]TTGCAGCCGTTCTCGCTCTGTGGCATTGCTCTTTGCCTTCTTGGTC | | |
| | CAGCTGCCTCCAGC | | |
| cg06140118 | CAGCAAAGCTGCCCTTAGACGAGAGCTTTCGCACCGCTGGCGCCTCCTGTTGCGC | −1.47922517 | Hypomethylated with Age |
| | GCGCT[CG]ATGGAGAAGTGGTCCCGACGCGCGCGTCGACTCTTCCAGCCTTGAG | | |
| | AGGCTAGCGGCGCG | | |
| cg06161600 | GAAAGCCGCCAAGGTGGCGCCCATCTGAGCGACAGGAGGGAGCGGCCCTGGCAGG | −0.00597085 | Hypomethylated with Age |
| | ACGGA[CG]TGGGAACTGCAGGGGCACAGGCCCTACGTGAGCTGCGTGGGTGGAA | | |
| | ACCGAGGCTGGGAC | | |
| cg06178942 | CGGGGCTGGCCAGGGGGGAAGGGAGGGGAGAAGAGGGAGCCGGGCGTCTCAGCGC | 0.0062742 | Hypermethylated with Age |
| | GGGAG[CG]GGTTTCAGGGTCCCCGGGCCCCTCCTCGCGCCCCGCCGCTGACTAT | | |
| | AGGGGGGGGGCCGC | | |
| cg06208270 | CCGCTTTCCAAAAGGCTTTAGTGGAAAACAGGTCCAGGGTGGGCCCAGTGGAGTG | −0.06242546 | Hypomethylated with Age |
| | GGCCC[CG]GAGGCATGGGGCACGGGGCTTAGGAGAATATTCGGATGGCTTGCGT | | |
| | GGCTGTGATGTGGC | | |
| cg06271623 | GCTCAAACACACTCAAGCCCCAGGACACACACTGGCACAGACACGTACATGCATC | −0.1751082 | Hypomethylated with Age |
| | CTGAG[CG]CTGGGACTCTCACGCATGCCACCTGCCATTGCAACGCCCTCCCAGC | | |
| | TGAGCCAGGGGCCT | | |
| cg06361510 | CTTCCGGCGGCGTGACCTGACCGCAAGAGGCCAATGGAGTGTGGGAGCTGAAAGG | 0.18713293 | Hypermethylated with Age |
| | GTCTT[CG]CTGGCGGCCGGTAACTGGGGGGGGTTGGGAACGGCCGAGTGTGGCT | | |
| | CTTCTGGTGTTTCA | | |
| cg06364315 | CCGTTAGGGCTGGGAGCCGGCTGGGCGCGGGGGTAGTGAGGGTGCCTCTCGCCGT | 0.49773059 | Hypermethylated with Age |
| | GGCTG[CG]CGGCGGCCTCGTCCGAGAGGCCGGCGCCGGGGCAGTGACCGGCCCG | | |
| | TGCCCAGCCGCCGC | | |
| cg06385324 | GCGGTTCCCCATCCCAGGGCCACCAGGGCCCCCGGGCCCCCCCGCTGCACCGGCG | 1.7098064 | Hypermethylated with Age |
| | TCATC[CG]CCATTTGCTGGGAAAAGCGACAAGAAGGAACTAGTCAGTGTGGCCT | | |
| | ACGCATCTGGCAGC | | |
| cg06516331 | TTCACTGGGCCCTCTGACTGTCCCAAGGCCCCCGCCGCCACTCCAGCGCCGTGCA | −0.02711672 | Hypomethylated with Age |
| | GCCAC[CG]CCGCACAGCCACCGTGGCCACCACCATGACGACTGCGCGTCCCCCT | | |
| | CGCAGGTGCGCCAG | | |
| cg06533408 | GTCTTTTTCCAAAAATAAAAAATAAAAACATGCTTTCAATAAGTTCTTTCCCCCC | −0.00969179 | Hypomethylated with Age |
| | TCTGG[CG]AGGGCTACTAAATTTGCTCAGCATTTAATACGTAAAATTGGCTAAC | | |
| | AGTGTCTGCACAGC | | |
| cg06595927 | CCTGCCGGGCCGGGGGGGGGGGGGCCGCTGGTAAACAGGCTGGGTTCTGGTGACA | −0.46121124 | Hypomethylated with Age |
| | CCGGG[CG]GCGGCGGAAGGCGGCCCGAGGGTCCCGCGCGTCCCACAACCCTCCA | | |
| | GTCCCGCTCTCCTG | | |
| cg06608166 | CTGAAGTTCCCAGGAGAGGCAAAAAATGAGGCTGATGAAGGTGGAAGAGCCTGGA | −0.06664474 | Hypomethylated with Age |
| | TCATG[CG]GATCTTTGAATGCCAAACTGAAAAGCTGGTTTTATTCTCTGGATGA | | |
| | CGGGGAACCAGTAG | | |
| cg06651180 | TGTCCCATGTCAGTTAGCAAGCCACCAAAGTCCATAAGGGATCCTGTGGGGTGGA | 0.08581398 | Hypermethylated with Age |
| | AGGTC[CG]CGGGGCCTGCTTCCCTGTTGCTGGTGCAGGCGGAGTGTCTGAAGGC | | |
| | TGCACGCATCTGGG | | |
| cg06667732 | AACAAGGGCTCCAAACCTAGTTTCAGAGTCTGACACACAGGAACTTTCGTATACA | 0.01864092 | Hypermethylated with Age |
| | GCACC[CG]GTTATACACAGCTTTCTCCCTCGTCCGCCGGATTCAGTGTCTGTCG | | |
| | TTATTGGGTTCATA | | |
| cg06691520 | GATCCCTCCCATCTCACAGTACCTCACAGGTCTCTTCCCCCGAGCAGTGCATTGC | −0.09409881 | Hypomethylated with Age |
| | TGGAG[CG]AGGAGAAGCTCACGAATCAGCTGCAGGTCTCTGTTTTGAAAAAGCA | | |
| | GAGATACAGAGGCA | | |
| cg06704773 | TCGGGTCACGGCCCTTAACAATAGCTTACTCGGGTGACTCGGCATGTGCCACCAT | 0.80894974 | Hypermethylated with Age |
| | CAGAG[CG]GTTGGCATTCATCATTACTCTCAGATGTCCCTACCAACACAGGCTT | | |
| | CATCAGAGGCAGGG | | |
| cg06729642 | CGCTCAGCAAACGCTTTACTTGCACAAGCTCTTCATAGCAGGCCTCTGCAAACCA | −0.10495364 | Hypomethylated with Age |
| | GCGGG[CG]CCGGGGAGAAGGGCTGCTTCTTCACTAGAGTTGGCGGCGAGGGAGC | | |
| | CCGCTTCGAGGGGG | | |
| cg06759629 | GCGATGGGTCCCAGTCATTAACTGGCTGTCAGGTTCCTCAGATGATGGAGCTAAA | −0.04914853 | Hypomethylated with Age |
| | AATAG[CG]CGCTATAGATAGAAGCTTCTCCCACGCAGGCAGGCGCCGGCTGCAA | | |
| | ATGGAAGTGGGGGG | | |
| cg06818605 | CCTCCCGGGTTGGCCAATGAAAAGCTGGCACTGGGTCGGAGGCGCCAGCCAAGTG | −0.14005984 | Hypomethylated with Age |
| | GGGGG[CG]GAGCTTCCACCACCGGCCAATGGGGATCTGGCTTCGGGATGTGGGG | | |
| | GGGGTCCACCCGGT | | |
| cg06848185 | CTCGGTGGGTGGGAGTTGGTGGCCTCTCGCTGGTGCCATGGGACTCGCATGTTCG | −0.12417949 | Hypomethylated with Age |
| | CCCTG[CG]CCCCTCGGCTCTTGAGCCCACAGGCCGGGATCCTGCCTGCCAGCCG | | |
| | CGTGCGCTGCCGTT | | |
| cg06848589 | TCCCTGCCTGGCTGAGGTGGCAGCAGGGGGGGGGACGCGCAGCGCTATGGCAGAG | −0.05337537 | Hypomethylated with Age |
| | GGCAG[CG]GGGAAGTGGTCGCAGTGTCTGCGACCGGGGCTGCCAACGGCCTCAA | | |
| | CAATGGGGCAGGCG | | |
| cg06897927 | GCGGCCCCCGACTTTGCGCCCCGTAGTTGAGTTCCGTTTATGGTCTGATTTCCGG | 0.01599462 | Hypermethylated with Age |
| | CCTCT[CG]CCTGCTCGCCCCGCCGCCCGCCTGTCCCGCTCCCTCCCTCCCGGGG | | |
| | ACCCGGAGGAGAGG | | |
| cg06922248 | TTGCTAAACCGTAACCCATTGTTCCCGCTGTTAACTCATGGACATGCCGCGTTTC | 0.13289426 | Hypermethylated with Age |
| | ATCCA[CG]CTGAACGGTAACCCGTTGTTACTACTGTCTTTTTGTTTTGTTTTGT | | |
| | TTTGTTTTTTTGAG | | |
| cg06937717 | CAGCGCCGGCCGAGGGCCCCAGCGGAGCTCGGGGGGGGTGCGGGGCGGTTCCAGG | −0.05752049 | Hypomethylated with Age |
| | AGCCT[CG]CCCCCTGCTGGGGACCCAGCTTGTGCCCTGGCGTCGTGGCCGCCGG | | |
| | CAGGCAGCAAGGAA | | |
| cg06951477 | CGCACTGCCATAAGGAGCTTCATCCAACCCTATGAATAAGCTGTTACACTTCCAT | 0.17762356 | Hypermethylated with Age |
| | TTTAC[CG]ATGAGACGCTGCAAAGTTGAGTAACACAGTCGCAGCGCTCATTGGT | | |
| | CCATTGGGTAGCCA | | |
| cg06975196 | TTATCCCCATTTTTCAGAGTGAGGGCTGAGGCCTAGTGTCTTGCCCAATGTCACA | −0.09144431 | Hypomethylated with Age |
| | AATGG[CG]AGGTCAAAAATCGACAGTCTCCAGAGTCTGCTCTCTTAACCACTTA | | |
| | ACTATTCTGCCTTA | | |
| cg06989443 | TAAAAATGGCCCAACCCATTACATTTTCTTTTAGGTAGATGGGGGAGCTGGGGGG | 0.0060779 | Hypermethylated with Age |
| | CGGGG[CG]GGGGCAGTCAGGGAACAAACAGCTGCCCTTAGAAATGACACGCCCT | | |
| | GTGGGCAATGGCGG | | |
| cg07025583 | TCGTTCTGGGCCTGAGGCTGTGGTAGCAGCAACACCTGCTCTGGCTTCACCTGCA | 0.10346304 | Hypermethylated with Age |
| | GCAGC[CG]CCGCACCGCGGGCTGTAAGCCGGACGCCACTGCCTCCCCGCACGAC | | |
| | GAGGCCAAGGTCGC | | |
| cg07040834 | ATGACAAAAAAGAAAGAGGTTTCTCCTCAATCTAACGGAGCCATTAACATCTATT | 0.12481811 | Hypermethylated with Age |
| | AATAA[CG]CCGACAGGGTAAGTAACGGAGCCGCGCTCCTCGGGGTGGTCACCGG | | |
| | GCTGCGTGGTCCTC | | |
| cg07059148 | CGCGTTATAGAGAACTGCCCCCTCGCTGCCCCAATACCAGCGCCGGGGCCGCGAG | −0.02510691 | Hypomethylated with Age |
| | CCCGC[CG]CTGATTGGGCCGCACCGCCCGTGACGTTAGCCCGGACCCCACCCCT | | |
| | CCGGCGGCACCGCC | | |
| cg07059402 | CCAGTAAGTTTAGTCTTGTGAAGTCCGAACGTTTGAATAATTTACTCGCTGCAGG | 0.02700144 | Hypermethylated with Age |
| | CAAAC[CG]CCTACAACTAAATCCATCAGGCCCCCGTATCCGAATCTTCCTTCAC | | |
| | GCGAGAAGCCGGCC | | |
| cg07082267 | GCTCCTCATGTGAGAAGGACCATAGGAATCTCCCGTTTCACAGGTGGGCACACCA | −0.421567 | Hypomethylated with Age |
| | AGGCC[CG]ACAATGGGTCCAGGCTGCCAAGGGTGGAGCCGAGATGCAAAGGGGC | | |
| | ACCTCAGAGCCTGC | | |
| cg07099606 | GGCGTCCAGCAGAGGCCGGTCAGGGCAAGAATGCCCGACCCTCAGGGTCCTCCTC | 0.00563278 | Hypermethylated with Age |
| | AGAGT[CG]CTGCGGGATCACTTAGGCGCCTCCGGAAACAACACTGTCTTTGCAC | | |
| | TGGAATTTTCAAAA | | |
| cg07109238 | AATAGAGCAGCTCATGGGCGTATTTGCGCTAGTGTTGGGTGTTCCGCTGTGCTGT | 0.18129078 | Hypermethylated with Age |
| | TTTTC[CG]TCATGGCTCGCACTAAGCAAACTGCTCGGAAGTCTACTGGTGGCAA | | |
| | GGCGCCACGCAAAC | | |
| cg07158339 | TACAGGGCTTAACTCATTTTATCCTTACCACAATCCTATGAAGTAGGAACTTTTA | −0.15852113 | Hypomethylated with Age |
| | TAAAA[CG]CATTTTATAAACAAGGCACAGAGAGGTTAATTAACTTGCCCTCTGG | | |
| | TCACACAGCTAGGA | | |
| cg07213780 | AGAGCCCAATTAAGAACTTCCAGAGTTTAGAAATGACTTGGGTTGATTATGTGTG | −0.12049015 | Hypomethylated with Age |
| | CATGA[CG]TGACCTCACTAGACCCAGCACGAAAGGGAAGCAGGCCTGGGAGCCC | | |
| | TCCCCCTTGCCCTC | | |
| cg07217350 | TTAGAGAAGCATCCTGGAGTGGTTGTTGTTAGTAATACTGTCTGTGGAGCACAGT | 0.01729452 | Hypermethylated with Age |
| | AGCGT[CG]CAGTAGAAGTTAGACCAATCCCACCTAAGTAGTCAGAGAATCTTAA | | |
| | AAAGTAAAGCCCAG | | |
| cg07356483 | AAGACTTGGGAATCCACCATCGGAGAGGGAAGGAGCTAGGACTGTTTTCCCATCC | 0.05011221 | Hypermethylated with Age |
| | GTTGA[CG]CTTTTGTGACCATCACCCACTAGTCTGGCTTCTGGGCCCTTGACTC | | |
| | TTAGAATGATTTGA | | |
| cg07392449 | ACCCCACGCCCAGGGCCTCACCCACCCCCAAACGGCAGGAGTTCATAGGACCCGC | 0.2936402 | Hypermethylated with Age |
| | GGCCA[CG]ACTGCCCGCGAGCGCCTACCGTGGGCCACGCCCCCCAACGACCCAG | | |
| | CAGGGCAAGTGTAG | | |
| cg07534331 | TTAAAATCCTCTCTCCTGAAGTTGTGTGGTCCAGCCGTTTGCTGAAGGAGGAAGC | 0.1771068 | Hypermethylated with Age |
| | AAAGC[CG]GTAGTAACTCACTACATATTTGGGCAGTGGAATGAACCCTGGAAGC | | |
| | TGACAAAGTCGAAG | | |
| cg07537392 | AACCCTTCCTGGCCCCCTCCATCCTAACAAAGCCTGAGTCGAACACGAAAGGAAG | 0.03566402 | Hypermethylated with Age |
| | ATGGT[CG]CTGAAGCGAAGGGGAGTCATTTGTGTCCGTTCCATAAATCAAGACT | | |
| | GTCGCCTTTCGAAA | | |
| cg07589899 | GGAGAAGAGAAGACGTGCAGCCAGACACCTGCCGCCTTGTCAGGCCTGTGTCGCC | 0.24914888 | Hypermethylated with Age |
| | GCCTC[CG]CAGCCCGAAATCATCCTGCCCTCCAAGGCACCGCCCTGATGCTCCA | | |
| | GGTGAAGGCTGAAG | | |
| cg07618159 | GTCAGGAAGACTGACAGAGGCGGGCCCAGCGGCAGCGCTAAGTCCAGTCTGGGCC | −0.0577648 | Hypomethylated with Age |
| | GCATA[CG]CCGCCCGCGGCCAGGAGTCAGCAGGTCATCACGTTACAGCTGCAGG | | |
| | GGAGAGACCAAGAG | | |
| cg07739179 | GGAACCTACCTTGGCAGCAGATTAAAGACAACCCGCCACATTTAGTCTCGGCCCC | 0.0513715 | Hypermethylated with Age |
| | ATGAC[CG]ATAGTGGGTTCAGTTCCTCCAGGGGGGGGGGAGCCTAGTGGCCCCG | | |
| | CCCCCTGACTCATG | | |
| cg07770857 | CCTTGCTCCGCTCCACGAGGAGGCCGCCAACCGCAGGGCCGCGACACGGACGGGA | −0.61663143 | Hypomethylated with Age |
| | AGCAA[CG]GACACTCTCCCAGCAAGACGCGTCTAGAGAAAGACCGCGTTTCGGT | | |
| | GCGGGGGGAATTTA | | |
| cg07815799 | GCGCCTGGCCCGAGTTTGTCCCGCAGGCTGCAGGCGACAGGACTGCAGGGCCGGC | −0.03439106 | Hypomethylated with Age |
| | AGGAG[CG]GGGCACACGGGGACCTCAGGGGATCTTGGTAGCCGAGGGCCTTCCT | | |
| | CTGAGAGCTGCAAC | | |
| cg07869795 | GTGGCGCCCTGAGCTGCTCAGTTACCAGAGCCGTTGGGGCCGATGATGCAGGTGA | 0.02820719 | Hypermethylated with Age |
| | ACCTC[CG]GAAGGGGCCAATGACCTGGCGGCCCCGCCACGACTTGAAATTTTCC | | |
| | ACAAGCAGCAGCTC | | |
| cg07876788 | TGGCAGGCGGCCTGGGCCTCTTCCTCTCCTATGTGTGGAAGTGGGTCAGGCTCTC | −0.01464599 | Hypomethylated with Age |
| | CCTCC[CG]GGGCCTGGGTTTCTAGCTCTGGGCAGCGCCCAGGCCTTACTCATCC | | |
| | TCTTGCTTATAGCC | | |
| cg07978591 | GTTGGCCTCCTGGGCACAGGCGTCGGACACCTGCAGGAGGTAGGCCAGGGCAGCG | −0.05160759 | Hypomethylated with Age |
| | CGGTG[CG]CGGGACCAGTCAGCCTGGCCAGGGCCCCGCTCTCCATGGCCTCCTC | | |
| | CACGCTGCGGGCCA | | |
| cg08097417 | CCGGCTAAGTCATGTTTAACAGCCTCAGAAATTATCTTGTCTCCGCGTTCTTTCT | 2.47905976 | Hypermethylated with Age |
| | TCTGC[CG]GCGAGCCAGGTAATGGTAACAGAGCGAAACTCCCCAGTCGGAACTT | | |
| | CTGGGTTGCAGCAG | | |
| cg08143133 | GCATCCGGGCAGACAAAGCCAGAAAAGCCTAGAACAGGATGCAGAGTGGTAACAT | −0.02495919 | Hypomethylated with Age |
| | TAGAG[CG]CACCTTGTCATGCTGGCCACTGGGTGGCAGGGGCCGGTTTCAGCGA | | |
| | AGGTACTCACACCC | | |
| cg08158862 | TTGCAGCAAACCACTTCAAGAGGGAGGGAATAAAGCCTGCGCTTGTTTCTCTACC | −0.11919296 | Hypomethylated with Age |
| | TTAGG[CG]AAGGTGACATTTTGGAATTTAACTTCATAGGGATTTAAAAGAAATT | | |
| | CTAAACTGTCACCT | | |
| cg08176056 | ACCCCAGGGGACCGGCTGAACGAGCGCGTGGCCTACCACCGGCTGGCCGCCCTGC | 0.06960841 | Hypermethylated with Age |
| | AACAC[CG]ACTGGGCCATGGCGAGCTGGCAGAGCACTTCTACCTCAAGGCCCTG | | |
| | TCGCTCTGCAACTC | | |
| cg08223357 | AAAGCAGCAGCGTCTACAGTCTGCCTTTATGTCCAGCGGGTGAAAGCCAGAAAGC | 0.01319193 | Hypermethylated with Age |
| | ACAGA[CG]GAATCTAGCCGATAGGGCTCCATGCTCTGCAGAAAACATCCTGACC | | |
| | CGAGGCCTGCAGGC | | |
| cg08231710 | CCCCGCGGGACGCCGGTGCCCGGTCTCGGTCCCAGCCCAGAGCCGCTCGCGCCTG | 0.13129877 | Hypermethylated with Age |
| | GACGC[CG]GCCGCCCCGTCGAACCTTTGGGTCTCCGAGCTCCCCGCCCCCGCCC | | |
| | CCAATCAGGACCGG | | |
| cg08279008 | CCTGAATGTAGCAACAGAAAGGGAACAGGAGGGGCAGGGGCAGAGAAGCCTCCCG | −0.19269377 | Hypomethylated with Age |
| | TCCCA[CG]TAAATAATTACAAACAGAGCACATGACCCCTGGCGGTTTCTGAACG | | |
| | CGCCTGGCAACAGC | | |
| cg08282512 | ATTCCATATTGCAACTAACCTTTAAGAAGTCAGCACCTGTTAGTGGAACCGCGAC | −0.00535951 | Hypomethylated with Age |
| | TGCTC[CG]CAGAGCTGCTGGTATGAGCGCCCGTCGCCACCCCACATCCCAGGCC | | |
| | CAGCCATTCTGACA | | |
| cg08301181 | AGCGTGCGGTGTACCTCCTCCTTAGCAAAGCTTTCTCAATGCCTCTTAGGTTAGA | 0.00270965 | Hypermethylated with Age |
| | CCCGC[CG]CAGGGATGAAGGGGTTGCTGGCGGATTGCAGGTGCCTGCAGCACAG | | |
| | GGCCCAGAACTAAG | | |
| cg08360726 | GCCTGTCCAGACAGAAGCTGGGGCCCACCGGAGGTAGCTGCAGACGCCTGAGAGC | 0.09916843 | Hypermethylated with Age |
| | GAGGC[CG]AGGCCCCTCAGGGGTAGGTGGGGGGAGGCTGGCTGGGGGGATGGGC | | |
| | AGCGGGGTGGCAGG | | |
| cg08439970 | TGTTCTGGCGGCAAACCCGTTGCGAAAAAGAACGTTCACGGCGACTACTGCACTT | −0.14293184 | Hypomethylated with Age |
| | ATATA[CG]GTTCTCCCCCACCCTCGGGAAAAAGGCGGAGCCAGTACACGACATC | | |
| | ACTTTCCCAGTTTA | | |
| cg08564027 | GTCTCAGCCTCTCAGCCTGGACTGGACAACTGGGCTTCGGGAATTCATTTAAATT | 0.01123985 | Hypermethylated with Age |
| | CTACC[CG]CTACACGCCTTCCCTGGATTCAGGGGGGCGTCCAGTGCATTCATCA | | |
| | CGCGTGTGCTGCCG | | |
| cg08596308 | CGTCGCTTTCGTCGTTACTTGTCTGCAGGACGGCTGGTCCGGGCCCAGGCCCTCC | 0.29894969 | Hypermethylated with Age |
| | TCCAG[CG]ACACCCAGGCCTCGATGTAGATGCTGGGCAGCCCCCACCACTGCAC | | |
| | CGGGCCTGTGCCCG | | |
| cg08611689 | TGAGCGCTTTAATATATATTAAATGGTGATAAATAAGGGGTCCAGGCAGCCGGCC | 0.0074868 | Hypermethylated with Age |
| | TGACA[CG]GCATTTGTCTTGGAGGGAGCAGAGATTGATATCTCGTGGGTGGCAT | | |
| | TAAAAACTCCCGCC | | |
| cg08701134 | CTCTGTAGGTACAAGTCAGGATAAAGGCGTTGTTTACTCCTGAGGCCCTCCCGCT | −0.31389906 | Hypomethylated with Age |
| | GCGTC[CG]AGGCAGCTGCTGCTGTAGTTCTGTCAGGGAAGGAAGGCGGGTAGCG | | |
| | GTAGCAGAGTTTGA | | |
| cg08779706 | CCGAGGCATGAGCGGGGGAAGGTGACCAGGACTTGGAATTTCATAAACGTCCCCG | −0.10550835 | Hypomethylated with Age |
| | TCAGG[CG]TGACGGGTCGTCAGGGCTGCTATCAAAGTCAGTCCGCCCATCTACC | | |
| | CTCAAACAAGCCAC | | |
| cg08822715 | GGAATGAACATGTTGGAAATAAACGCTCTCATTTTGCAGGCAGATAAACTGGGAA | −0.04010952 | Hypomethylated with Age |
| | TCGTG[CG]TGTAAAGCAGCTTGCTCAAAGTCTTATAACTATGAATTGGAAAGTC | | |
| | AGATTCGAGCTAGG | | |
| cg08859206 | TGAGTCCGCTTTCCGGCCAACCCCTCCTCTCAGTCTTAGGCCCCACTGCAAGCCT | −0.0035471 | Hypomethylated with Age |
| | ACTCC[CG]CTCACCGTACAGAACCTAAGCACAGGATCAGAGATGGGGACAGGTT | | |
| | GACTCAGTCCCATG | | |
| cg08861270 | CGCCTCGAGTGCCCCCTCGCGCCCAGGGGTGGGAGTACAGAGCCAGGCTCGCCAT | −0.03763155 | Hypomethylated with Age |
| | TCCAT[CG]TATTAGGTCAGTAAGATTGACAGGCACGATACGTATCAATAACATT | | |
| | GCTGTGCACAACAC | | |
| cg08903425 | CAAACTCCAACTATATTCTTGGCTTCCCTCCCTCCTCTGGTGGAAGGAATGCAAA | 0.04237108 | Hypermethylated with Age |
| | ACTGT[CG]ACAGTTCTGGCTATTGCTACCTGTTTGCACTGTCTGTCCCAAAGGT | | |
| | CTCCTCGACTTGCA | | |
| cg08924488 | GCAACGAAGGCCGCGAGAGTCGAGTGAGGGCTTGAGTCTGGTGGGGGGGGGAGTG | −0.10374004 | Hypomethylated with Age |
| | TCTCC[CG]CCGCCGCGCTTGTGCCGCCGCTTCTCCACACGTGCACTCGGGTCTC | | |
| | TCGGCTCCCTCCCG | | |
| cg09093137 | TCCAAACCCGAGAGCCGAAACGCACAGGTCTCGGGGCTGAACTCGCGCCAGGAAC | −0.10361476 | Hypomethylated with Age |
| | ACGCC[CG]AGGCAAACCACTTGACAACCAGCTTAGGTTCTCAGCAGAAAGGCCG | | |
| | ACAGGCGGGGGCCG | | |
| cg09096950 | TTTTATCTGCCCTCGGTACGCTGATTTCCAAAACCCAGCCTCATATTCTATACTC | 0.01808794 | Hypermethylated with Age |
| | CAAAG[CG]CACTGCCAGGTGGGCCAACTCCAGCCCCCACAATCCGATGCCAAGG | | |
| | CCACTTCTTGCCAC | | |
| cg09189118 | GAGGTGGCGTCCCTGTCCCCAGCCAGGGGGCAGCGCGAAGCTGCCTCCCCGCGGG | −0.3322831 | Hypomethylated with Age |
| | GGGAG[CG]GAAGTGGCCCAGCTGCTCGAGTGACTTACTAGTTAAAAAGCTGGGG | | |
| | TTGGAGCTGCCACG | | |
| cg09244436 | TTTGCTCTTTAGGCCAAAATACCAAACCTAGACATCCTGGCTATCTCTATTCTTT | 0.05936366 | Hypermethylated with Age |
| | AAGAT[CG]TTCATGCAACTAATGCCCATATTCTGAAGACCCAGGTCATCATGAT | | |
| | TTGACCACCATCTT | | |
| cg09265397 | CCCCCGGCGGCGACCCCGGGAAGCTGCGGCAGGAGGGTCCCGACAACCCTGGGGG | −0.10488754 | Hypomethylated with Age |
| | GCAGG[CG]CAGCGCGGCCCGCGGGGCGTCTGCTGGCATGGGACGCCCACCGGGC | | |
| | ACTGCAGCTCCCGG | | |
| cg09274827 | TTCCCGCGCCCAGAGGCATGGATCCCAGGCCCTGCATTTTCCCAGAGAATGGCGT | −0.05784798 | Hypomethylated with Age |
| | TGGTC[CG]GAGAGGGAGAGACAGGAGCCTGCAGTCACACAGCAGGGTGGGCCAG | | |
| | GTCCCGTGCAGCCG | | |
| cg09281539 | GGTCCAGCACCTTCTGGGTGGACTTCTTCACATCCCCGTGGCTCCTTCGGGAGAA | −0.353392 | Hypomethylated with Age |
| | CATCC[CG]CGGCAGGAAGCCCGGGCCCCGCCGGCGGGGCAGTAGGCGCCTGCGC | | |
| | CACGCGAATCAAAG | | |
| cg09510128 | CTGCTTCTGTTTCGCGGATGTCCGGGAGGTGCAGTGGCTCGAGGTCACGCTGGGC | −0.00600039 | Hypomethylated with Age |
| | TTCAT[CG]TGCCCTTCGCCATCATCGGCCTGTGCTACTCCCTCATTGTCCGGGT | | |
| | GCTGGTCAGGGCGC | | |
| cg09661809 | TTCTTCGGGCTGGTGCTGGCACTCATCGGCCTCATCTTCCTCATGGTGCTCTACC | 0.11906573 | Hypermethylated with Age |
| | TAAAC[CG]CCGCGGCATCCAGCGCTGGATGCGCAACCTGCGCGAGGCGTGCCGG | | |
| | GACCAGATGGAGGG | | |
| cg09680131 | CACACTGCAAAGGCGGGTGCTTTCAAAATTCACTTTTTCCCACAAGCGGATTCAG | 0.10847269 | Hypermethylated with Age |
| | AAATG[CG]GAGGTTGCCTGCCGCCTTTTCTACCGGACAACATTCCCGAGTTATT | | |
| | GGTGAGCTGGAATG | | |
| cg09687864 | CCGTGCTGCCCCAGGCAGGTTCCCCCACAGAGGTGTCCTGTTGAGATTCCTCCCA | 0.00606884 | Hypermethylated with Age |
| | TCAGA[CG]CCGCTCCCAGAGCTGTGGCCCGCAGCCCTCCTGGGGCGCCTCCTGC | | |
| | CCTGAGCTGAGGCC | | |
| cg09748749 | CTGGCACATAGAGGTGCCTGGTACGTGTTTGTTGAATGAATGAATGAATGAGTGA | −0.19295944 | Hypomethylated with Age |
| | ATGAG[CG]AACATGCCATTTCACCTTATATATCTTGTGAACCTGCCAGGCCCGG | | |
| | GCCTGATGTCATAG | | |
| cg09766323 | ATGGGGATTAGAAACAATTCATGTCAAGTGCTTGGCACCTAAGAAGTGCTCAATA | −0.0344199 | Hypomethylated with Age |
| | AATAG[CG]ACTGTACCACACCTCCTAGGAGCCCTCAGCGTACTGAATTAGAGTT | | |
| | CTCTATAAGTCTCC | | |
| cg09809672 | CCCCAGAGAGCTTTCATCTAGAAGGTTTGACTCTGGCCAGACAACCAGCGAGCAT | −0.1587779 | Hypomethylated with Age |
| | CTTCT[CG]CAATCTGTTGCTTCTTCCATGGCAAACTCCAGAGAATTAAGAAGCC | | |
| | AAACTCAACATCGC | | |
| cg09829551 | GGATCTGATTATTGAGGTGTGGAAGGAATAAATAATCAGTCCACAAATAAACAAA | −0.02361329 | Hypomethylated with Age |
| | CTGTC[CG]GGATTCCTAGAGGGAAGGAGAAATCCTTGAAGGAGATCCAAGTCGC | | |
| | TCCAGGTCTGCCTG | | |
| cg09894698 | CAGCGTCTCCACCTTGCTCAGCTTCTTGCTGGCGCCGCCGTGCGGCACGTGCTGC | −0.16414989 | Hypomethylated with Age |
| | CGCAG[CG]CCTGGAAGCCCAAGTTCACCAGCTTCACGCGGTTGCGCTCGCGCTC | | |
| | ATTGCGCCGCGCTA | | |
| cg09898978 | TGCTTGAGGCGGTGCTACAAATGAAGTCCTTCTCCAAATGCATTGAGCCCCAAGA | 0.02983162 | Hypermethylated with Age |
| | AAAAT[CG]CTGATTCTGAGGAACCTTCCAACTTTAACAATTTCAGGACGTCCCT | | |
| | AAGCTGAGCTAGGA | | |
| cg09931450 | GGGCCAAACACCGCTCAGCCTGGTACCCCTAAGACCTCACTCAAAAAGTCAGGGA | 0.32929045 | Hypermethylated with Age |
| | GTTTT[CG]GCCACTTCAGAAAGACTTTGTTGTTACCCCTCCGTCTTAACCCAGG | | |
| | TGAGTTAAGTGACA | | |
| cg09971754 | CGTCGAAGAGAACGGCCCTCGGGCGCGTCGCGGCCGCGGCTCCAGAGCCCCTGGG | −0.19878859 | Hypomethylated with Age |
| | CCTCG[CG]CTCTGAGAGCCTGAGCCGCTTGAGGAGGCCAAGCGCCCACGAACCG | | |
| | CATTCCCTGCCTTG | | |
| cg10086328 | TCTGCAGAACAGCCAGGAAACAAGCCCGGCCCATGGCGCCACCTGCTGCCTGCCT | −0.20938803 | Hypomethylated with Age |
| | GCCTC[CG]CTCATGCACCCTGGGCTGGGATGGTACTTCTGTTCGTCTGGCATTA | | |
| | TTGCCCTTGGTCAT | | |
| cg10091994 | AAAGACAGCCTTGACTCAAGCATGCGTTAGAGCACGTGTCAGGGCCGACCGTGCT | 0.67894404 | Hypermethylated with Age |
| | GGCGG[CG]ACTTCACCGCAGTCGGCTCCCAGGGAGAAAGCCTGGCGAGTGAGGC | | |
| | GCGAAACCGGAGGG | | |
| cg10115490 | GAGGCGGGCAGCGGGGGCCGCGAGGGGGGGGACTGGCGGCGGCGGCAGCTGCGCA | −0.44012708 | Hypomethylated with Age |
| | AAGTG[CG]GAGTGTGGAGCTGGACCAGCTGCCTGAGCAGCCGCTCTTCCTTGCC | | |
| | GCCTCACCGCCGGC | | |
| cg10164885 | CCCCAGCCAGCCCATGCTGCCCCGAGGCGGGAGCCATCCCTTTCCCCAGCCCCAG | 0.05901067 | Hypermethylated with Age |
| | CTCTG[CG]GCCACTCACCTGCTCCGAGACCGAAGATCAAACAGAATGTTCTCAG | | |
| | TAAGACCCGAGACT | | |
| cg10215507 | TATTTGAGCTCAAACCAAGCGACTGTTGACTTTAGCACACAAAGCAAAGATTTCA | −0.03913891 | Hypomethylated with Age |
| | CTGCC[CG]CTAGTTTAAAAATGAATATTTTACCAAGATATCGATCAGCGTTATA | | |
| | AAATTCAGTTAAGT | | |
| cg10258962 | GCAGTGAGCTGAGATTGTGCCACTGTACTCCAGCCTGGCAACGCAGAGGTTGCAA | −0.00837092 | Hypomethylated with Age |
| | TGAAC[CG]ACACGGTGCCACTGCACTCCAGCCTGGGCGACAGAGTGAGACTCTC | | |
| | TCAAAAAAAAAAAA | | |
| cg10308673 | CGCCTACCGCCCTAGAGCAGGAAATAGCGGTCAGCGCCAGCTGTGAGGAGCACAG | −0.29599771 | Hypomethylated with Age |
| | CATTG[CG]GCCAACACAGGAGGCACTGACCACGGGGCAGGCGCTATTTAAAAAT | | |
| | CGTGGCAAAGGATC | | |
| cg10364115 | GCAGCCTCCTGGGAACACAGCGTCCCATTCCCAGGGGCTCAGCGGGCTGGCGGGA | −0.02614468 | Hypomethylated with Age |
| | GGGGG[CG]GCGGGGGCCGTGGGTTTTGTTTGGCGGCCGGGCCGTTAGGATTCCC | | |
| | AGCGCCGGGGGGCT | | |
| cg10381888 | GTTCGCCTACGATGGCAAGGATTACATCGCCCTGAACGAGGACCTGCACTCCTGG | 0.17977449 | Hypermethylated with Age |
| | ACCGC[CG]CGAACACAGCGGCTCAGATCTCCCAGCACAAGTGGGAAGCGGACAA | | |
| | ATACTCAGAGCAGG | | |
| cg10523019 | CTCGCTGCTTCTCCCCTAGTCTTCGGGTCCCTTGAACGCAGGTCGCTTGTTTGCC | 0.0328151 | Hypermethylated with Age |
| | TTACG[CG]TAGTCAGCGGCCAGTGGCTATTTATGGCAGTAAGGAATATTATCCA | | |
| | CATTTCACATGGAG | | |
| cg10596537 | GCAATGCAGGGGAGCTGGAAATAGGCAGAAGCTAGATCAGGTGGTGACTGACGTG | 0.2506423 | Hypermethylated with Age |
| | GCAGT[CG]ACTAAAGGAGTTTCTTCAGGTTTCTTTGTAAAAGGCAACGGCAAGC | | |
| | CTTTGAGGGGTTTT | | |
| cg10668512 | CGGAATGTGCCATTTGGACCGGTCGGCAGCAGCTACGGTTGCCGGTCCCGCACTG | −0.29491684 | Hypomethylated with Age |
| | AAAAA[CG]ACAGTGGTGACGGGTGAGCTCCCAGAAGCAGAAGAATGACAGGCAA | | |
| | CACCTGAAGCCACG | | |
| cg10699215 | TCGCGCCTGCCCTGCGCGCTGTGGTTGCGGACGCCCCGAACCCGGAAGCGCGGTC | −0.00010827 | Hypomethylated with Age |
| | CCGCG[CG]CGGCTCGCCCCCAGCTTTGACCATATATAGTCAAGCGCTCGGCTCG | | |
| | GCGGCTGCGGTCCC | | |
| cg10767425 | GGCCGTCTTCCTCCTCTTTCCTTTCACCCTAGCCTGACCGGAAGCAGAAAATGAC | 0.07312865 | Hypermethylated with Age |
| | CAAAT[CG]GTATTTTTTTTTAATGAAATATTATTGCTGGAGGCGTCCCAGGCAA | | |
| | GCCTGGCTGTAGTA | | |
| cg10812186 | CTCCACGTGGTAGGTGGTGCCGGGCCTGAGGTCGGGCAGGCTGACGGTGCGCGTG | 0.09211973 | Hypermethylated with Age |
| | GTGCC[CG]GCACAGTCAGCTCACCGCCGGGGCCCTCTGCAGGCGGCTGAGGCCG | | |
| | CCAGCGCAGCACCA | | |
| cg10935612 | CACACACACACACAAGGCTCCTCCGCAGGGGACTCGGGGGGAGATCTGCAGGTGG | −0.00155793 | Hypomethylated with Age |
| | GTGC[CG]TGGGGAGGGACAGCTGCCTGCTTGTAAATCCGCCCCCTGCCTTCTTC | | |
| | TGGGCTGCCTCTC | | |
| cg11018337 | GCAGTGGCTCAAAGGACCGAGCGGGCGGTGCAGGTTGGAACCCGCGGGGGGACCA | 1.3199712 | Hypermethylated with Age |
| | ATCG[CG]GCTCGGCCACAGCCTCGCCCGCTGATTGGTCCCTCCAGGCCCCGCCC | | |
| | CCGCTCGCCCCGC | | |
| cg11051055 | CTCCACACCTGCCCCATCTGCAGCAGGTGGATGACCGACTGCCAGATCCACACGG | 0.41663908 | Hypermethylated with Age |
| | AGAGG[CG]ACACAGGCGCTGCGCCCCCGGCGTGGGGGAGACCCTCACGCCTGGG | | |
| | CCACCGCGGGCCGC | | |
| cg11084729 | GGTGGGACTGGGGTGCAGAACCTAAGATCTGGCTATTGTGTTCATGGCTATGTGG | −0.06879547 | Hypomethylated with Age |
| | TGACC[CG]CTTCACCTCACAGAAGAAAGAGCTGACTCCCCAAAGAGGGGCTGGA | | |
| | GGTCCCCCTAGTCC | | |
| cg11108890 | TCCGACTCTACGGACCCAGGTCGCTGTGGCCCATCGCTTTCGATTTGACTTGGTT | −0.04009183 | Hypomethylated with Age |
| | TCTGT[CG]CCACTCGCGGAAGGCGCGCCCCCCGCCCTCGCTCGGCGGCCCGCCC | | |
| | CGCCCCGCCCCTGC | | |
| cg11176990 | TGCCCAAGAGCGCTACGTCGCCGGGGGGCAGCAGCAGCGCCTACAAACTGGAGGG | 0.74177436 | Hypermethylated with Age |
| | GGGGG[CG]CAGGCGCACGGCAAGGCCAAGCCGCTGAGCCGCTCTCTCAAAGAGT | | |
| | TCCCGCGTGCGCCG | | |
| cg11197015 | GCGGTCGCCGCCGCGGCGGGGCGGTGGGCCGGGTTCTCCTTTGAAGGGGCGGTGG | −0.14427235 | Hypomethylated with Age |
| | GACCGG[CG]GACTCTCTGGGCACTGGCTACCACGGAGACGCCGCTACGCTTCGG | | |
| | GGGGGGCCCGTCTT | | |
| cg11198128 | CCCTTAAAAGCTGGGGCCTGGGACAGGAACGACAGACAATGCAGCCAATGGCGTC | −0.57754179 | Hypomethylated with Age |
| | ACGCG[CG]GTGCCCCGCTACCCAATCGAAAGGCGTGGCTGAGGGAAACGCGGTG | | |
| | GGAACCGCCCCCGA | | |
| cg11212038 | TCCTCCTCCCTCTTCCTGGGGGTGCTGCTACTTCCCCGGCCTTGTGTGCAGGACT | −0.24761676 | Hypomethylated with Age |
| | GGGGC[CG]CCGTTACCTTTCCTCGACCCACCAGACCCCTCAACCACACAACCCG | | |
| | AGACGAATTCCCGC | | |
| cg11267527 | GGAACTCCGCTGGTGGGAGTAGGTGTCTTCTGTGCATTTTTTTTCCAAAACCACT | −0.00570273 | Hypomethylated with Age |
| | TTGGC[CG]TTAGATGGCTGTGGGCCGGCACTCCATCCATCCATCCATCCATCCA | | |
| | TCCACCCACCCACC | | |
| cg11299854 | CCGGGAGCTGGGTTATAAAATGCCGGGTTAAGCGGCAACTCAGACTCAGGATCCC | 0.00411447 | Hypermethylated with Age |
| | GCTCA[CG]ACATGGCCTCGGGCGCTCAGCTCCCGCCGCAGCCGTCGAGCTCAGA | | |
| | GGTCAGCGCCGTCC | | |
| cg11324538 | TGGGGGCCCAGGGGGTGTCAGCTCGGGGCCTTGCCTCTTGCAGCTACTCTGTGGT | −0.05700374 | Hypomethylated with Age |
| | CAGGC[CG]GGTCCTCCACCATCAGGAAGATCCCATCCTGAGCTCTGTCTCCTGC | | |
| | CCCTCCTGCTGTGG | | |
| cg11388238 | GGTCTTGTGTGTTCAGAGGCTGGTTTTACAGGTGAAGAGAAGAAACAGCCGCAGA | −0.18891105 | Hypomethylated with Age |
| | AGTTG[CG]ATTGTCCAAGGTCACTTAATAAGTGGCAAGAATTAGGATGTTAAGT | | |
| | GTTCTCACCCCCAG | | |
| cg11495430 | CGGAGATTCCCAAGTCAGATTCACAAACACATGGGGCGTCCTGGTGGATAAACCT | 0.05357644 | Hypermethylated with Age |
| | TTCCC[CG]GAAGACACATTTGTGAAGAGTCTTGGCCCCCAGTGTTGAGACTGAT | | |
| | TCGGCGTCCTGAAA | | |
| cg11530693 | CCCAGACGGCAGCCTCCCGCGGACCCAGCCCCTAACACAGGTGCAGCTTCTGGTG | 0.00198631 | Hypermethylated with Age |
| | CTGCG[CG]AGGTGCGTTTTATAGCGGAAGCCTTTGCCGCAGCCCGCACACTTGT | | |
| | GCGGCTTGTTGCCC | | |
| cg11586600 | CGCAGCGGCGTTTCATTAGAGCCCCGGGCCCGGGCCGCGCGCCAGGAACTTCCCC | −0.25409187 | Hypomethylated with Age |
| | GCACG[CG]GCGAGATCGACGATCCCCCGCCCCCAGCCCCAGCCCGGCTCCAGGC | | |
| | CTCGCAATGTCAGG | | |
| cg11614451 | GTCTGCAGGCAACATTCAACTGCAAGGCATCGGCCAATGGGAACTATTGCTGGGC | 1.30578415 | Hypermethylated with Age |
| | TCGTT[CG]AAAGTAAACGGTGGACGGCGCGGCCCGAGGCAGGTGGGGGGAGTCA | | |
| | GTTTAAGGCTGGCG | | |
| cg11731114 | AGGTGACAATGACAACAAAATTGACGCGGACGCTCCAGTCAAAGGCATCTCCCCT | 0.09668367 | Hypermethylated with Age |
| | TTATC[CG]ATGACTCACCCTCTTAGGAAGTCGGCCCGAGAGGCAAATCTCAAAA | | |
| | TACCTTGACATGAA | | |
| cg11834844 | ATTAACTCATGCTCCTAGCTTCGCTAGGAATGTGATAGAGAATTTCCCCTGTAGG | 0.12470646 | Hypermethylated with Age |
| | TTCTT[CG]TATGGTGCGCTCCGCTGGATCACGTGAGCCAGTTCCAAAATGGGGG | | |
| | CAGGGGTGGCCGGG | | |
| cg11850549 | TCCTGTTCCACCTACGTAGGATCTGTGAAACAGGCTCAGTGCCTTTGAGGGAGGA | −0.06374098 | Hypomethylated with Age |
| | GGGAA[CG]TTTAGATTGAGACCACCCCACTCCCGGGTGATTAAATAAATATGTC | | |
| | TCTCCCCCACCCCA | | |
| cg11999255 | AACCTCCTCTTTGAAGCCCACAAGATACGGTGAGGATTACTGCCTTTTTCTGATT | −0.0225573 | Hypomethylated with Age |
| | TCCAA[CG]TGGGTTTTTCATTAAGCAAAGAACAATTAGAAAACCCACACATAAC | | |
| | ATAGGGTATTCAGT | | |
| cg12117135 | GCCACACAGGGGGAAAGGAAGCAGCTGGAGAGTCCGCTGCCCACACCACCCCGGG | −0.06833284 | Hypomethylated with Age |
| | CTCCA[CG]GCCTCACCCCCAGGCCGTCACAGAGCTCCAGTCTCCCGCCACTTCA | | |
| | GTGCAACCCTCGCT | | |
| cg12119029 | CTGGAGGACCTTCTTACCTGGGGGAACCCGTCTCACCTGGAAGACCTTCACCTGG | 0.08817205 | Hypermethylated with Age |
| | GTAAT[CG]CCGTGGCCTCCCACTACGGCGCAGCCGGGTCGGCTGCCCGGGCTTC | | |
| | ACCCTAAAATAAGG | | |
| cg12181372 | CCAGGGTCACCCCCGAACCAACAAAGCACACACACACCCGAGATGCGCCCCAGGC | 0.11523262 | Hypermethylated with Age |
| | CGCAG[CG]CCAACCCCCCTTCAGATGTCCCGGAGACAGGCGAGCAGCGGTCCCC | | |
| | AGGGCTCCCGTCGC | | |
| cg12379463 | GGTCAAGCTCACCCTGACGTCAGGGCCTTGTTGCTCCCTTTACATGAACTTTCTT | 0.0375078 | Hypermethylated with Age |
| | CCCCA[CG]AGGCAATACAGGACTCTCTCCCACCCTTGCTTCAGGTAGCTGCTTG | | |
| | AATTTCACCTTCTG | | |
| cg12454161 | CCGGGGGGATCACTGCTGTTGTCCCCCACCCAGATCTCCTGAGGGTCCGGCAGGA | −0.10731369 | Hypomethylated with Age |
| | GGTGG[CG]GCTGCAGCTCTGAGGGGCCCCAGTGGCCTGGAAGCCCACCTGCCCT | | |
| | CCTCCACGGCAGGT | | |
| cg12492345 | GAAACGCCTACCTTGGCACTTAGGGACCAGAAGCCTCTGGATGTCTAGCAACAGG | −0.35910248 | Hypomethylated with Age |
| | GGTCA[CG]GGATCACTGCGTGGGGTCTCTGTAAGCAGTCCCCTGAGGCAGTGCA | | |
| | AAACCGGAAACCTG | | |
| cg12589526 | GGCGGCGGCGGCGCCAACTGTTTTCAAACAGTGGCGGACAAACAGGGCTTGGGGC | −0.06266212 | Hypomethylated with Age |
| | TGGCC[CG]CACGCTGCCTGATCGTTTCCGCCCGCCGCTCCACCTCCCCGCGGGC | | |
| | CCCGCACCCCGAGA | | |
| cg12664038 | ATCAGGCTGCACATTCAGCATCGACAGCCCTGGCCAGTAGGCCTTCTGGGCTCCC | −0.00072584 | Hypomethylated with Age |
| | AGCAA[CG]CCTGCATTTTGCACATTGGTTTGCATGACTGGTAATGTCCCTTCAC | | |
| | AGGGCCACCCTCAC | | |
| cg12683944 | GGAAGGGTCCGGAGAGGGGCCACAGGCTCCTGGCCTTTCTAAGCACACCAAGTGC | −0.08929245 | Hypomethylated with Age |
| | CCAGT[CG]CGGACCCCCGGGACCAGGATGCGCTGACGACCCGGCTGGCAGGCGG | | |
| | GTCCTCGTGGGCGA | | |
| cg12688884 | AGAGACCTAGCGCAGAGCCCAGGTGGAAGTTCCAGGTTACCCCAGACCTGGCCTA | −0.08670219 | Hypomethylated with Age |
| | GGACT[CG]GCGCTCTGAGCCACCGCAGCCAGTCTTTTATGCATCCGGGGGGAGT | | |
| | TTCGGTTTCCTTTC | | |
| cg12695586 | TGAAACAAACCGGGAGGGCCGTGAGGAGACCGCCGCGTTTCTCTTCCGACGCGGG | −0.0452576 | Hypomethylated with Age |
| | TAGGG[CG]TGCTTGTCCCATTCCCAGGAACCCAACTCATCTGAAACAACAGGGC | | |
| | ACAACCGCCGGCCT | | |
| cg12768993 | ACCAAAAACAATAAACCTTTTATGACTTAACCAAGGAAGCACAAATTATCTCCAA | 0.11119885 | Hypermethylated with Age |
| | AGAGG[CG]GAAAGCAGGCCTTACAAGATCCAGGACCACCCCCAAAGACAGTTCA | | |
| | AAGAAAGCAAAGTT | | |
| cg12772971 | GCGAGTGGTCTGCGGGCAGCAGCTCCCAGAGGCAGCCTTGGAATTCCAGCTCGGA | 1.3865914 | Hypermethylated with Age |
| | CTGGG[CG]GGAAGGCGCAGGCGGCCCAGGTCGCCGACACGCTCACGCACCCTCC | | |
| | CTGCCTGGCCGCGC | | |
| cg12848614 | TTTCCCTGCTGCCATGCCCTTTGGCAGGCAGCCGTCCCCACGCCCGGAAAGCCCC | −0.04274408 | Hypomethylated with Age |
| | AGCTT[CG]GCTCAGCCCACAGCACAAGGGCATATCCTTCTGCCTGCGCAGGCCA | | |
| | GGGTGCTCGACGCC | | |
| cg12854815 | GCAGGCACCTCCAGGCCTGGCTGGCAGCGCCCTGGGAGTGGTCCCCTGGTACCTC | 0.03337008 | Hypermethylated with Age |
| | TGCGG[CG]GAAAGAGGGTAACAACAGGCTTCCTATCTGAGGCTAACCCCTAGGT | | |
| | CGACGTACCAGGTC | | |
| cg12897901 | CTTGATTCTCATTGTTCCCGGGAGCGAGCGCCTTGGCTGCGCTGGGCATACCCAC | −0.16284943 | Hypomethylated with Age |
| | CCTGG[CG]CCATTCACAGGCAGTGCCTGCCCTGGCCCTGTGCTCACCCCATCAG | | |
| | GCCTCCTCTTCCGC | | |
| cg12955789 | TGGGGCCGGGGCCATCGCCGTGGCGTCTTGAGGACCTGACGCGAGCTCTGTGGTC | 0.00267618 | Hypermethylated with Age |
| | CTGGA[CG]CCAGCCCAGGGAGGGCAGATGTCTGCACTGACTGGGATCCGGGGCT | | |
| | GTGAGGGGGGGGCT | | |
| cg13103209 | GCCTTGCCGCCAGTCTTGCCGCGGCCCGACATGCTAGCGAGGTAGACCGGTGAAG | −0.54449437 | Hypomethylated with Age |
| | CACGA[CG]GCTCAAACACTAGAACAGACGCCCGCCGCAGTGTAACTGCTGTCGC | | |
| | GCGCGCGCCGCGAG | | |
| cg13147090 | CGCGGGCCCCGGGCCCGGGGATGACGCCGCGGAGACCCCCGGCCTGCCCCCGGCC | −0.04065258 | Hypomethylated with Age |
| | CACAG[CG]GGACCCTCATGATGGCTTTCCGGGACGTCACGGTGCAGATCGCCAA | | |
| | CCAGAACATATCCG | | |
| cg13174651 | GCAGCGACTGTGGCGCGCGGAGTCCGAACTGCAGATCCCGCTGCCGCCGGAGCCC | −0.40495653 | Hypomethylated with Age |
| | CGCCC[CG]CGTGGGGCGAGCTCCCCAAGCTCCGCCTCCAGGCTCCCCGGCCTCT | | |
| | CCCCACCCTTAAGC | | |
| cg13187936 | CCAAAGCTCCCTCTACACCAAGCTGCTCCCTGTACGGCAACAGAACTCCTGGAAC | 0.32668443 | Hypermethylated with Age |
| | TTGTC[CG]CAGAGAATGAGCAGGGCCCTTGCTTTTTTATGGTCTTGGCCAGGTT | | |
| | TAGCCTGCCTTGTA | | |
| cg13351161 | CAAAGTCACTCAGCTCGCCAGGGGCAGAGCCAGGGTGCTCACAGGGTGGCCAACC | −0.00089766 | Hypomethylated with Age |
| | TTCCA[CG]TCTGCCCTGGACACGGGACTTTCAGTACTAAAATGTGCGGACGTCC | | |
| | TTCTCCCGGACGTC | | |
| cg13398440 | CATCCTCGCCAGTAACCAAAGAAGCGCAAATTAGCGCAAAAAGCAACGCCGCTTT | 0.08981945 | Hypermethylated with Age |
| | GCTGC[CG]GAGCAGCAAAATATTAAACACTAAAGCCCCCGTGCTGGTGAGGATT | | |
| | TGGAGAAACCAGGA | | |
| cg13494498 | ACCGGGCCTCCGCAGGTGCAGCTGGGAGCCCTGTCTGGGCGTGGCCCTCTTTTTT | 0.33299147 | Hypermethylated with Age |
| | GGGGC[CG]CGGCATAGCCTTGAGTGAGACGGGTGGGGTAGGCAGGTTTGGGGGG | | |
| | GGGGGGGTTCTC | | |
| cg13575298 | CCGAGGGTGAGCTCGCTGGCAGAGGAACTTGCGCCTTCCAACTCTTAGGTTTGTT | 0.01114509 | Hypermethylated with Age |
| | GCGTT[CG]GGTCGCAGTCGACTTTAATGGGAACCAAGCAAAGCACCCAATGGCC | | |
| | CCTGCCATCAGGTG | | |
| cg13577297 | GAGGACGTCGAAGTGCGCACTAGCCCTCCATGGAAGACGGGAGCAGAGGACGGTG | −0.00267936 | Hypomethylated with Age |
| | GAGTG[CG]CACCAGTCCTCCGTGGTAGACAGGAGCAGAGAATGCCTGGAGTGTG | | |
| | TACTAGCCCTCCAT | | |
| cg13579112 | AGCGTGTCCCGCTTGAGGCTGCTGCCCTTGTTCACCACCTTGATCCTGAGGGCCA | 0.26235876 | Hypermethylated with Age |
| | GTTTC[CG]GACGCTGGCGGGCCCCAGGCCGTCGAAGAAGAAATCCTCGTTGAAG | | |
| | ACGGGGCGGCGGCT | | |
| cg13612447 | GGGCCGCTGGGCTCGCGGAGCTGGCTTTGGCTGGTAGCTGGAAGGGCAAACTGGG | 0.22291818 | Hypermethylated with Age |
| | GAACC[CG]CCGGTGCCTTGGCGCGCAGGGGCGCGGCGAAGGATCAGCACCGCGG | | |
| | ACAGCGCCCAGGCC | | |
| cg13617776 | GCGCGCTGGGCACTGGCGGGGGGAGGGGAGGGGAGGGGGGGGCGGAGCCGTTACC | −0.16116668 | Hypomethylated with Age |
| | AGGG[CG]CCCGGCCCTGCCCCGGGCAGTGCCACTGTCCGATTCCAGGATGCCGA | | |
| | GTGGCTGCCGGTG | | |
| cg13855261 | CCCGTAACCCCAGCCAGCACGACATTCAGACACCCCTCCAGGCCCAATTAGCTTC | −0.03414989 | Hypomethylated with Age |
| | ACAGA[CG]CTCAAGACTTGGGAAAACAAAAGAGGAGAAGATAACTGAACCCCTC | | |
| | TCCCTGTGCCCACC | | |
| cg13875111 | GGAATGGTTAGTGAGCCCAGGCAAGAAACTCATCAGCCGCATCCTTCAGAACACA | −0.07885314 | Hypomethylated with Age |
| | CTGGG[CG]TCCTGAACTTAGGCTTCTGGGGACAGACACCTACCCTCGATATTGT | | |
| | TATTCCAACGAGCC | | |
| cg13933409 | GCCTTTCCCCGCAAACCGCAGGTCCGGCGAGGACTCGGGACCCCGAACTCACCCA | −0.64073296 | Hypomethylated with Age |
| | GCTGG[CG]AGGGAGAAGACACCCAGCACAGCCCCCATGGTGACGCCAGTGATGG | | |
| | AGGTGGCCGGTCCT | | |
| cg13983442 | AGACAACTAAATTATGCACACCCCACTTTGTTTTATTAGCTTATTAGCTACTCGC | 0.05614695 | Hypermethylated with Age |
| | ACTAT[CG]ACTGTGTAGAAGTGCAAACACTTCTCAGCCCCAACCATAAACTGCT | | |
| | TATTTATAAATAAC | | |
| cg13992856 | AACTGCACTGCCCAGAACCGGGTTTCCCAACCTGTAGAGGCCGCATCCGCGTCTC | −0.53446716 | Hypomethylated with Age |
| | CTGGG[CG]GGCAGTGCCGGTAATCCCCAACAAGCCCCAGCCTGCTTGGAATTAA | | |
| | CGGGTCTGACTGTG | | |
| cg14001239 | TAAAGGCAGAGCTGAGAAGATACACGTGCTGCAGAGGGCGTAGCCCTGGAATAAC | −0.05163784 | Hypomethylated with Age |
| | ATGCA[CG]ATCTCTCATCCCTCCAGATCCATCTTCGGTTGTGAATCCGGCCCGG | | |
| | AATGCAGCCACCCC | | |
| cg14030282 | GGCGGGCGCTGCCTTCCAGGGGTGAAGTGTTTTCGGACCCCGGAATCTGTGGGCG | −0.21714067 | Hypomethylated with Age |
| | GCCTG[CG]GGAGGGGCTGAGGCGCAGTTCCCTACTCACCCAGGTCCGAATCCAC | | |
| | CGCGGTGCTGTTTC | | |
| cg14037250 | CACCCCCATTGCCCAGCCACCCAGGTGAGAGGCTGTCGGGAAAGCTGCTCAGGCG | 0.00131386 | Hypermethylated with Age |
| | AAGCG[CG]ACCAGCTCAGAACGACTCTCCAACCACTGCTCTCAGGCACGGGAGG | | |
| | GAAGGCCTGGGCGG | | |
| cg14074174 | TGGGGTAGGGGCCTTGGCAGTCACTGGGACTCCTAACACCGGGCCAGCAGGAGAC | −0.29837261 | Hypomethylated with Age |
| | TAAGG[CG]CAGTAGCGGGGCCCAGAGCATACAAGGGATTGGGCTTTGGCTTCTC | | |
| | TGCTGCAGCCCTGA | | |
| cg14089881 | CACTGCACTCCAGCCTGGGTGACAGAGTGACACTCCATCTCAAAAAAAAAAATTT | 0.02095952 | Hypermethylated with Age |
| | TGAAA[CG]GCCGCACCCTCGCCGGCCCTGCGTCGTCCCCGAAAACCAGACGCCC | | |
| | TGGGGCGCGGGGCC | | |
| cg14097171 | TCGTGATTATATGTCCCAAATAACCCGTAGAAATAATAACTGTCATGAAAGGAGA | 0.069934 | Hypermethylated with Age |
| | AGCCA[CG]TGCTCTATTTGTCCACAGGCTGAGGACCACCTTGCTGCGGTTGACC | | |
| | GCTGCTGGCCGGGA | | |
| cg14128973 | CAGTGGGAGGGGTGGGTGGAAGAAGGCTGGTCTCTGTCTGACCAAGCCCCCCCAG | 0.00129139 | Hypermethylated with Age |
| | AATAA[CG]CAGGCTGCCCCCCTAGGTGGAAACAATGACACAATCAGCTCCCAAT | | |
| | ACCAAGGGCCTGAC | | |
| cg14135988 | GGACCGCCCGGCCTTGGACCCATCCGGAGCCACAGGTTGGAGGAGATAAGTAGCT | −0.13374569 | Hypomethylated with Age |
| | GTCCC[CG]TGCTCATCGCCCTGTGGAGCAGATCCTGTCTCCTTGCTGACGGTGG | | |
| | AGCCCGGGAGTTCC | | |
| cg14147842 | GCTTAGGAGAGAAATTGGCCACGATGAATACACTAGAGGTAATACTTTAGAGTTT | 0.42709734 | Hypermethylated with Age |
| | TGCTG[CG]AAAGGCGGCAAGGAAAAAGGATAGTACCTGTTAGGGAAAGCAGAGT | | |
| | TCAGATTCTTTTAG | | |
| cg14194875 | CTATCCCTGTGACAGGAAAAGGTACGGGCCATTTGGCAAACTAAGGCACAGAGCC | −0.4138026 | Hypomethylated with Age |
| | TCAGG[CG]GAAGCTGGGAAGGCGCCGCCCGGCTTGTACCGGCCGAAGGGCCATC | | |
| | CGGGTCAGGGGCAC | | |
| cg14295611 | TTTGGAAAATGAGACTACCACTTGGCAATTTGGTGTCCTCATTCCACTGCATCAA | −0.19910419 | Hypomethylated with Age |
| | AACAC[CG]AGAAGCAGGGCCAGGCACGGTGGCTCACGCCTATAATCCCAACACT | | |
| | TTGGGAGGCCAAGG | | |
| cg14305711 | TAAACTTCCTGAAAAAAAGGATGACAGGTAAGGATTAGGCAGAGATTAAATCTGA | −0.36387726 | Hypomethylated with Age |
| | GTGAT[CG]ATCCTATTCAGTTAATGGGTTGGCAAGTCCTGGAATGAGATACAGC | | |
| | CATCTAAAAATTAA | | |
| cg14330189 | TCACTCTCAGCTTTCAAAAGCAGAATACTGTTCTATTAGGTGTTTCTCTCTGCAT | 0.20895729 | Hypermethylated with Age |
| | GTTGT[CG]GCAGTGTTCTGAATAGGAGGAGATCCCTGCCTTTAAAGGGGGCGAG | | |
| | ATTGGGGTGGAGGG | | |
| cg14339760 | CTGCCGGCCCTGGGGCATTGAGCCTCAGGAGGCCCTCGGGCTCAAGGGGCCCTCC | 0.25099814 | Hypermethylated with Age |
| | TGGTG[CG]CTCTTCTTCCCAGGGGAGCGGGACTACGGCCCCCCCATTGACCTGT | | |
| | GGGGTGCTGGGTGC | | |
| cg14409507 | TATTCAGAGCCAGGCAGCAGGAGGGAGCTTTGCCCCAGAGGAAGCTCAGCCATGC | 0.03164128 | Hypermethylated with Age |
| | TGTTA[CG]GAGAGGGCGCGCTCCCCTCGATGCACCAGCCGTTGTCAGAGGAGGC | | |
| | CCACGGCAGCGGGC | | |
| cg14446107 | CCTGGCCTGCCCGGCCCGCGTGGTGTCCCAGTGGCTGCGGCCACGCCAGGCATTC | 0.06535085 | Hypermethylated with Age |
| | TGCCC[CG]CGGCGGCTGCACAGGGACGAGAACTGAGAACCCCTGCTCAACCCCA | | |
| | TCCGGGGTGACTGC | | |
| cg14502172 | CAGTCCCGGGAACACACTTGCATAACCTTTGGTAATTGGAAATATATCTCATATT | −0.03444127 | Hypomethylated with Age |
| | GGCCA[CG]TGCACAATAATTCAGTGTGAATATGGCCAATAAACATGCCTTGTTT | | |
| | ACAGGTCATTAGTT | | |
| cg14535884 | CACAGGTTAGCGGCAGACTTGATCCCGAGTCTCCTAACTGGCAACCCAAGACTCT | −0.0699313 | Hypomethylated with Age |
| | ATCCC[CG]GAACTGGCAAGAATCTTCCTGAACTACCCCGATAAAATTTTGAGTG | | |
| | CCAAAGAAAGTCCC | | |
| cg14550076 | AATAGTGGAAAGGAAAGTCGTGGAGAAGGCCAGCACCTGCCCGGTGTGGGGAGCA | −0.01790051 | Hypomethylated with Age |
| | GGGCC[CG]GGCACGTGAACCTTTCCCTGCGGAGCTGGTGCCTGTGGGTGCACGG | | |
| | GTGTGGTGCGTTTT | | |
| cg14556482 | GCCGCTAGCCGTACCCCAAAGTGGGCAGAAGCCCATGAGGGGAAGGTGAGGCACC | 0.07188137 | Hypermethylated with Age |
| | TGGGG[CG]GAGAGAAAAGGAAAAAACCTTGCCACGGAGAAGGGAGGCCTGGGTT | | |
| | CCCCATGAAAGAAA | | |
| cg14604336 | TTAGACACAGTGGGGGTTGAATGGATGCGCGCGATATAAGCAAATTAAATGGTTC | −0.00420926 | Hypomethylated with Age |
| | GTGGC[CG]CATCCTAGAGCCCTTTAATTAGAGTTACCATTTGTAGAAGGCCTGC | | |
| | CGTGCACAATCCCT | | |
| cg14640772 | AATGTTTTCCACACCGCGTGTGAAATGACGAGAGCTTTTGTGTCACACATGTAGC | −0.16480379 | Hypomethylated with Age |
| | TGCTA[CG]AGACAGACGCTCTTGTTCCGGATCAGAGCAGCAGACAGAACCGGCA | | |
| | CTTTTAGGGTCCAA | | |
| cg14829814 | AAATATGAGTTTGGTAAGTCCTGAGCTCCCGTGATAAGGACTTGGTACTGGTGAG | −0.04709819 | Hypomethylated with Age |
| | GATGC[CG]ATGTTGAGTTGCATGACAGCTGTTTCCACTCTGCTTAAAAACACTC | | |
| | AGCTCCCTCCTAAG | | |
| cg14895961 | TCAATTGTTTTGTGAAGGGAAAAAAATCTCAATTTGCTTTGGAAGGCTGGGAGAC | −0.00061986 | Hypomethylated with Age |
| | AATAC[CG]CAATTTGGGGGCTCCAGGAAAATCGTTTTTGAATCTAAAGATCTAG | | |
| | GGAAGTTGCTCAGA | | |
| cg14917329 | GGTTGGCTGGCCCTCTGATATGAGGTGGTGGTGAATTGGTGGGCGTCAGTGCAGT | 0.01618016 | Hypermethylated with Age |
| | AAGTGAG[CG]AGCCACGTCGATTGACTGGTCTTCAGAAGCAGGTTCGGTTTTGT | | |
| | TTTGTTTTGGTTTG | | |
| cg15022387 | CCTCCCCCTCCCCCCGAGGCAGCTGATTGGCTGTTGAAAGTTCCGCCCCTCCTAA | 0.13983999 | Hypermethylated with Age |
| | TTGGC[CG]TGGCCCGTGCCGTGGCCCGCCCCTTGCTGTGTCTCCTCTGATTGGT | | |
| | TGCCTTTGAGGCTG | | |
| cg15033511 | AGGAAGGAAGGAAGGAAGGAAGGAAGGAAGGAAGGAAGGAAGGACACACGTAAAT | −0.00855108 | Hypomethylated with Age |
| | CCCAG[CG]ATTTTATTTAATTTTGAGACGGAGTCTCGCTCTGTCTTGCCCAGGC | | |
| | TGGAGTGCGGTGGC | | |
| cg15035273 | GGAAAACAATAATGGCCAGTTACCCACAATTGGGGCGGGGGCAGGGGAAGGTGAC | 0.03436114 | Hypermethylated with Age |
| | GGAAA[CG]GCTAGTTACCCAGAATTCTCTGGGGGAACCAGAAAAATCGGTTATC | | |
| | TAGAATTCTCCCAC | | |
| cg15065069 | GAATTCATGTCAAGCTATCCCTCCTCGGTGGAGACTCAGTTTCTCCACTTAGGGA | −0.17441637 | Hypomethylated with Age |
| | ACCCG[CG]GGATGGAGGTGGGGAGCAAAAGCGTCGGCGCGCCCCCTCCCCCTTC | | |
| | GCAGACTGGGCCCC | | |
| cg15076218 | GCGAGCGCTCGGGGGCTCAAACTGCCTGGAGACAGGCGGGGCTCCTGCTGAGGCT | −0.00377708 | Hypomethylated with Age |
| | TGGTC[CG]CCCGGACGAGTAAGGAGAGGAGCCAGGGAGGACCTGCTGGCGTGAG | | |
| | ACGCCTCTGCCCGG | | |
| cg15090185 | TAGATCTTATATTACATACCAACTCGGCTATTCATTTAAGGGGGTGATAGCCCCT | −0.20142959 | Hypomethylated with Age |
| | TTTCC[CG]CCCCTCTGCACAAAGCTTGCAGGGGAGCTTATAATGAAGGAATGAA | | |
| | GCAAAGTGTCCCAT | | |
| cg15108984 | TCCTACCCCCAGGGCTGGCAGGGCAGAGAGAGCTGATGAAATTGGCTTGGGTGGA | −0.25146004 | Hypomethylated with Age |
| | GTTCC[CG]CGGTTACAGATAAGACCTTGGGGACAGGTAGTCCTCAGTCCCCAAA | | |
| | TGCTTACGCAGCGT | | |
| cg15131414 | CGCCTGGGGTGGAGCGGGTGTGTTCCGCCGGGCTCCGGGATGCACTTGCGCAGTT | −0.18618486 | Hypomethylated with Age |
| | TCACC[CG]AGGCTGGAGTGACGCCAACCTGTTAATGTTCGTTTTCGGATTCTGG | | |
| | ACTTCGGGTTCCGC | | |
| cg15171982 | CGGCGCCCCCGCGAGGCCGGACGGGGAACTGCGGCCGACGGGCCCTCGGCATGGC | 0.14964749 | Hypermethylated with Age |
| | CTCGC[CG]CCGCCGCTCTGGCCCTGGCCCTGGCCCGGACCGCCCACTACGCCTG | | |
| | ATGCCGGGATGGGA | | |
| cg15212354 | CACCTCGGCCCCCGACGGAGCCGGCACAGAGCAAGAATGAGGTGGTGCTCATCCC | −0.16845762 | Hypomethylated with Age |
| | ACACA[CG]ACGTTCCCTGTGCTGGCGTCGAAGCACCTGCACAACGTTGCTTCTT | | |
| | TGGGAACACGCATT | | |
| cg15257096 | CTCGGCCTCCTCGTCAGTGACAGATCTGTACTGCACCCCTCACAGCAGTAGGTCA | −0.37772476 | Hypomethylated with Age |
| | GACCT[CG]TCCTGCCCGGCACGGCCGGGGACTTCAGCCTGAGCGCCAGCCTGTC | | |
| | GGCCTGTACGCTGC | | |
| cg15300101 | CACATGTACCCTAGAACTTAAAGTATAATAATAATTAAAAAGAAAAAAGAAAAAG | −0.02114058 | Hypomethylated with Age |
| | AGAAA[CG]AGGGTCTTGCTCTATTGCCCAGGTTGGACTCCTGGGCTGAAGTGAT | | |
| | CCCTCTCCTTCTCA | | |
| cg15324873 | AACACAGTATCTGTGACACAGTAAGTGCTCAATAAATATCAGCTTTTATTTTTCA | −0.04483791 | Hypomethylated with Age |
| | AGGGG[CG]AGGAAGGTGTATTTTGAAAAAGCATATACTATTTATTTTTTTAAAG | | |
| | GCCTACAGAAAGTA | | |
| cg15363134 | CCTGGGCCCCTCTCCCGCTGCCCGGGATCTCTTCTGTCCAGGTCACCCCAGGCTG | −0.00763646 | Hypomethylated with Age |
| | GTCAC[CG]TCCGCCCCTGTCTGTGCCCATTCTCAAGGCCTCGTGTCTGCCCTCC | | |
| | CCGCGCGGAGGTGA | | |
| cg15382048 | AGGATCCAGCGCAGTCTACAGGGAAGCTGCTTTGCTTTCGATGCTGCCAGCACGG | 0.00745749 | Hypermethylated with Age |
| | CCAGG[CG]CCTCATTCCCAGCTCTCGCTCCCACCTCTCTGAGCCACAGACCCCG | | |
| | TGTGGGTGAGGCGG | | |
| cg15401405 | GTCTGTCTGCCCTCCCCTAGCTTCAAGTTTCTGGAGCCCCTCCCTTAACTTTCAT | 0.03352824 | Hypermethylated with Age |
| | GTCTC[CG]AGATTTCTCATTTCGAATTCTCAGGCTCGTCTGACACATTTCCACT | | |
| | TCTTTATTCCTACT | | |
| cg15401952 | GGGAGAGAGAGAGAGAAGGGAAAAGAGACTAGATGGAGATGGAGAGGCTGAGAAA | −0.04774526 | Hypomethylated with Age |
| | AAGGG[CG]TGAGTGACAGAAAATGCATTCATCCTGTCTGCTGTAGCAGAGGCCA | | |
| | GAAGTAGCAGCCGG | | |
| cg15466909 | TTATAAGTTTAAAGTAACAAGAAGGACCCTGAGCTCCCACGGAGATGATTCTGGA | 0.01086571 | Hypermethylated with Age |
| | AGCAG[CG]ATGCCCCCGGGTTGGAGCAGAGGAGCAGCGAGGGTTTTCTGCGCTC | | |
| | GGCATCGCGGCGGG | | |
| cg15477144 | GATCACACCCTTTACGGACGCGGCACCTGCGACAGGGATGCGCGAGGAGTCAGGG | −0.00436477 | Hypomethylated with Age |
| | GGCCT[CG]CCGGATCGAACCTAAGCTGGGGAAGAGTATTTCTTGTATTTTTAGG | | |
| | AGAAATTCTCAGCC | | |
| cg15481583 | GCTTGCATTCCAACCCAGACTTTGCTACTTTGTAGCTTTGACAAGTTATTTTTCC | −0.12725345 | Hypomethylated with Age |
| | TTTCT[CG]GTCTTAATTTCTCTGTGTGTAAAGTCAAGATAACACCAATGTGCCT | | |
| | TCCGGGTTAAAATG | | |
| cg15553989 | ATACGTACGTGTTAATGGTGCTAAATAACTGATCACCTGACACGGGGGGGGGGGG | 0.00059734 | Hypermethylated with Age |
| | CGGTG[CG]GAGAGTAGAAAGGAAGAGAATGTTGATATCGCGCACGGAGTCCTGT | | |
| | GTCCCTCCTTTCCC | | |
| cg15622309 | GGCTGTGCGGGAAGGGGGGGGCTGGCTCGGGTCCTCCTGCGAGCTGCGGCCGGCG | −0.15255105 | Hypomethylated with Age |
| | CCTAA[CG]TGCAGACCAGGCCCCAGGCCGAGGCTTTATCCTGGAACCACCAGTG | | |
| | TGAGAGACAGCTTT | | |
| cg15641675 | CGGAGACGGGAGCAAAACACAGAGAATCGGGGCTACAAAGCCGGTGGGCAGGTTT | −0.01244608 | Hypomethylated with Age |
| | GGCTA[CG]CTCAAACCGGGCAGTGCCGCGGTTTAGGCGTCTCCTTCCTTCCCAG | | |
| | CGACTGCACAAAAT | | |
| cg15652532 | CCTGATCAGGGAACCTGGGTTCTATAACTGCTTCTACTACTGATTTGTCCTGTGA | 0.00082917 | Hypermethylated with Age |
| | CTTCG[CG]CACCAAATTTAGGCTTGTAAATTAAACTCCCAGATTTCTGTTTTCC | | |
| | ATTTTGCAGCTCTA | | |
| cg15707455 | CTCAAGTGATCCGCCCGCCTCAGCCTCCGAAAGTGCTGGGATTACAGACGTGAGC | 0.13583836 | Hypermethylated with Age |
| | CATCG[CG]CACGGCTTAGTCCCAAGATTTTAGAGCCAAGAAGCGTTTGCAGGAG | | |
| | GACGTAGGGAAAGA | | |
| cg15711508 | ATGCAGATCAGTCAGGTTGACAGCTCACATTTTCTTGCAAAACTCTTAGGAATTT | −0.02029264 | Hypomethylated with Age |
| | TCCTC[CG]CAAATCCCTCCGCAGTCAGTGATAAGTACTAAATGCCACAAAGAAA | | |
| | ACAATTTCCCATTG | | |
| cg15718594 | GGTTGCCCCAGGACTATTTTGCATCCCACAAGGTACCCACACAAGGTGCCTAGCA | −0.06125059 | Hypomethylated with Age |
| | TAGCA[CG]CTACATACACTCAACAGGTGCTCAGTATATGTATTGCACTAGGTCA | | |
| | GCTGAAAGCTCTCA | | |
| cg15798279 | GCAGGATGACGATGGCCGCCAGGTGGGACAGGTCCCCAGTCAGCCGGAAAATGTT | −0.18024145 | Hypomethylated with Age |
| | CATGG[CG]GCGGCGGCGGTGGCGGTCGGCGCAGCGCGGCGGCCCCGGGGCTGGG | | |
| | CGGCTCAGGAGGCG | | |
| cg15829826 | CAGGCAGGGAACTAGCCCGAAGACCCAGCGTGGGCAGGCCAACGCCCCTAGCCTG | −0.63596878 | Hypomethylated with Age |
| | ATTCT[CG]AATCTTCGCCAAACCCTCTTACAGGCCAACAGTCGGGGCCTTTGTG | | |
| | AAGGGAAACTCGCT | | |
| cg15890274 | CCCTATGAACTCTGACCCCAGCTCCCACACTGTCGCCTATCACCGGCCCACTCTC | −0.02743397 | Hypomethylated with Age |
| | CGTGC[CG]CGTGTCCCTTAAAAGCTGGGGCCTGGGACAGGAACGACAGACAATG | | |
| | CAGCCAATGGCGTC | | |
| cg15936446 | CTGCTGCAAAAAAAACAACTTTTGGCGCAAAGAATGTTGCGGCCAGAGAGCATCC | 0.19025663 | Hypermethylated with Age |
| | GCTGT[CG]CTGACAAAGGAGTAGCAATGGCAATGAGAAACCGCCGGCGCCACGG | | |
| | CCGACCGCGGCGGC | | |
| cg16013006 | AAGAGCAACCCTGGGAGGCCGGGGGCAGCGCCAGGGGCAGGCGGCTCCCCCTTGA | 0.14919869 | Hypermethylated with Age |
| | GCCTT[CG]TCTGACATGGGCCGTTTGTGAAGAAGGGAAGAATGTGTTAGAGGGA | | |
| | GAAGGTGGTCACCG | | |
| | TTAGAGCTTGGAAGTTCCAAGAAAGACACAGATTCGTCCCCCGCTAAGCCAGCAC | 0.03752267 | Hypermethylated with Age |
| | TTTTA[CG]GGATCGCTGTGGGGCCGGATGGCTTCTAGACTTCCGGGTGATTCCG | | |
| cg16019898 | GGAGATGGGGGACG | | |
| cg16045271 | ACTGTTTTGGGCCAAGCCTGGCTTTCAAGAGTCCCCTCTAGTGGAGATAACCTGC | −0.29918112 | Hypomethylated with Age |
| | TACTT[CG]GCTTCTCAGTTCAGCCCAGAGCCTCCCTGGCCAGTGAAGATTCAGA | | |
| | AAACCCCCTTCTGC | | |
| cg16097124 | CAATAAATAACATTTTATTAATCACGGTTAGCTCTGTCTCCTTTACTTTCCTGCA | −0.06893003 | Hypomethylated with Age |
| | GGTAT[CG]CGGTATCTTATGATGCCAATCCGTATAAATATTCAACACCCCAACA | | |
| | TTTATTTGGAGAAC | | |
| cg16146501 | GGGGCGCTGGTCCACCGACTGGCCCGGCAGTTCCCCAGTAGCACCGGGGGTTTCC | −0.07416419 | Hypomethylated with Age |
| | TGCCG[CG]CGCGTCACCTGGTTCAGTTTAGAGCCTCCAAAGCTGCAAGGCAAGG | | |
| | CGAAGATGGGCAGT | | |
| cg16148346 | GCAATAAGATTTTCATGCACCATAAACTTTCCTGAGTATCTCAACCAGTTTTGTT | 0.16071167 | Hypermethylated with Age |
| | GATGC[CG]GGGTTTGTTCAAAGCTGCAGATTACTGGGCCTCACCCCAGACCTAC | | |
| | TCTACTTAAATATA | | |
| cg16179976 | AGCGAGCAACAGGCGGCGCAGGAGCTGGAGCCGGAGAACCTAGTGTGCTTAGCGG | −0.68959575 | Hypomethylated with Age |
| | TCGCC[CG]CTGGGTTCCTCGCGCGCCTGGCCGCCCCTCCCCTAGCAACGGCCCG | | |
| | GCCCCGCCCCGCCG | | |
| cg16273546 | GATTTTATCTATAGCTCAATCCAATATTTAATGACAACTTCAGAATATGATATGT | −0.00124468 | Hypomethylated with Age |
| | ATTCC[CG]ACACACTTGCTGACATGTTTTGGATCACTAAGATAATGAATCATTT | | |
| | ATATTTTGCGAATT | | |
| cg16311044 | CTCCGGCCAGGCCTTGCTGCGCTGACTGCAACACAGGCTGAAGGCTCCCCTGCCT | 0.01325283 | Hypermethylated with Age |
| | TCAGT[CG]GCCGAGTGACCTGCACCCCTCCCACAGGCCCGGTGACGGCTATGAC | | |
| | TGTGCTCCCGAACA | | |
| cg16411541 | CAGAACAAGCGCCCGATTTCAGGGGAGCCCAGCCGAAGGGTTCCACAGCGCTCAG | 0.0284443 | Hypermethylated with Age |
| | CGAGC[CG]GCTGGGAGGGAGCAAACTCTCTAACTGCAGAAAAAAACAAATCTTG | | |
| | ATTCCGCTTTAAGG | | |
| cg16465768 | TCTACAAATTTAGCCGTGCAATCTAATAGCGAGCAGGCCGAGACGCCCGCGATTG | 0.38117782 | Hypermethylated with Age |
| | AAGGC[CG]GGGTGGAGAAAGTAATAAGGGCCCTGGAAATTACCCCCATCCCATG | | |
| | TCACCCACTCTTTC | | |
| cg16480692 | CGCCGCCCGACAGCCACGCAGAACAGACGCGGCAGTGCGACGCCTCCCCCACTGG | 0.34123191 | Hypermethylated with Age |
| | GGACA[CG]AGACAGCGACAGCCACGCGGTGAGCCGGTACAAGGCCCTCTAGGCT | | |
| | TCAGCGGGTCTGGA | | |
| cg16551665 | GCCGAGCGGGGGGCGGGGCTGCGGTGCCTGCAGAACCTTGGACAGAAGCTCCCTA | −0.22947662 | Hypomethylated with Age |
| | GCTGC[CG]CCGCCGCCGCCGCCGCCGTCGCCGCCGCCGAGCGCGAGCCCAGCCG | | |
| | ATCCCCGCCGAGCG | | |
| cg16624692 | CTATCACCGGCCCACTCTCCGTGCCGCGTGTCCCTTAAAAGCTGGGGCCTGGGAC | −0.8363818 | Hypomethylated with Age |
| | AGGAA[CG]ACAGACAATGCAGCCAATGGCGTCACGCGCGGTGCCCCGCTACCCA | | |
| | ATCGAAAGGCGTGG | | |
| cg16674327 | GCCGGCCGCTGTCAGCTCCCTCAGCGTCCGGCCGAGGCGCGGTGTATGCTGAGCC | −0.01888078 | Hypomethylated with Age |
| | GCTGC[CG]CAGCGGGCTGCTCCACGTCCTGGGCCTTAGCTTCCTGCTGCAGACC | | |
| | CGCCGGCCGATTCT | | |
| cg16703882 | CCGGAAGGCGGGAAGGGGAACCGCTGCCGGGGGTCAGTCAGGTCGTTACCCTCCG | 0.34222334 | Hypermethylated with Age |
| | TCAGT[CG]CGGGCTGCCCGGCTCCCTGCTTCTCTCGGCGGCGCCCATGTCCAGC | | |
| | TCCCGGACGGGAGA | | |
| cg16714096 | CGAGACCCAGAAGGGTGAGAGGGGGAAGCCGCACCCTCTTCGTGGGTGCGATGCT | −0.00562174 | Hypomethylated with Age |
| | GTGAA[CG]TCTTCCTTCAAGGAAAGAGCTGCTTCTGGATTTTCCTTGAACTCCA | | |
| | GGAGGTGCTAAGAA | | |
| cg16767506 | GGGCAATCCAGGGCCCTCCTCGAGGGAAGCGGGGTTTGCGCCAGGGTCCCCAGGG | 0.03402167 | Hypermethylated with Age |
| | CTGTG[CG]AACACCGGGGAGCTGTTTTTTGGAGAAGGCTCTAGGCTGACCGTAC | | |
| | TGGGTAAGGAGGCG | | |
| cg16785344 | AGGCCTCCAGCCACTCCCATCCATCTTTCTGTCCCTCTCAAAGTCACTTGCCTGA | −0.09983794 | Hypomethylated with Age |
| | CCCTG[CG]GATGACAAATCCGTCCACAGTCAGCCATGTGTCTGTGCATTCGTCA | | |
| | GCCACTGGGTCATC | | |
| cg16949584 | ATCAGGTAAATCAGCCCTGGATAAAATAGCAGGAACCTGTTCCGCCGAAACCAAA | 0.03818203 | Hypermethylated with Age |
| | TGAGT[CG]TCAGGTAAATCAGCCCTTGATAAAATAACAGCACTTGTTGACCAGA | | |
| | AGCCATGGGACCAG | | |
| cg17101029 | TTGTCCCATTTTACAAATGGCGAGTTCTCTGTTTCTAGTCAGTAAATAATCAAGA | −0.07677287 | Hypomethylated with Age |
| | GGAGC[CG]GGATCAAGAACCCAGTCCACCTAGCTCCAGAGGCAATGTTCTTATG | | |
| | GCTTCAGTGACATG | | |
| cg17104388 | GAGGCCACCCTCAGTTGGTTGTCATGTGGCCCTCTCCATATTCTAACTCACAAGA | −0.06268025 | Hypomethylated with Age |
| | TGCTC[CG]ACCTCCCGCCTGTGCGCCCCAGTGGCGCGAACCGGAGTGCGCCTGC | | |
| | GCGCGGGCCACAAA | | |
| cg17163168 | TGGGCACCACACGTGAGGAGAGAGCAAAGATCCCGACCTGGGAGGCCCAGCGAGG | 0.06360031 | Hypermethylated with Age |
| | CCAGC[CG]TCCCCGCTGGACTCGTCAGCTGCTCGGCCCCGCCCACAGGCTGGCT | | |
| | GCCCCGCCCCGCCT | | |
| cg17233127 | AACCACCACCGCCGCCGCCGCCGCCGCCGCCGCCCGCAACCCGCCTCTCCCTACG | −0.0630703 | Hypomethylated with Age |
| | GGTCC[CG]ACTGGGCACCACTTCCGGTCCGACACGGCCACGTGTTACATCTAAA | | |
| | TGGCACCGTCCCCC | | |
| cg17239008 | TGTCAGGTTGGGGGAGAGGCCCAGGTTCCTCACCTGGCCTCTGTGAACACTTGAG | 0.01606202 | Hypermethylated with Age |
| | GGAGC[CG]TGCTCCTTGTTATTGCTAGGTGTGGATGGGGGTTCAGGCTTCCCAC | | |
| | TAGGTCTCTGCTGA | | |
| cg17330460 | AGAAGAGGGAGTACTGTCCAAAGGATTAGCAGACACGCACTATGATCACTAGAAA | 0.28315999 | Hypermethylated with Age |
| | GCAAT[CG]TGGTCTGTATGTAAGGCAATCCCCAAAATAATTTGAAAATTGCTGC | | |
| | CACAGCCTCCCACA | | |
| cg17401282 | ACCTCCCCCACTGTCTGCTCCCCCAGCTCTAACAATGCAACCATGATTTCTAAGA | 0.01523416 | Hypermethylated with Age |
| | CTGTA[CG]AGTTTAAAATAAGGATTCTTGGCAGATCTGACGACCACAAACCTTC | | |
| | TGTCATTGATGATG | | |
| cg17417004 | ATGCCAGCCTTTCCCAGGCACCTGCCACATGCAGGGCAGGTGGACAGACCAGCAA | 0.0746937 | Hypermethylated with Age |
| | TGACA[CG]GTACAGTGAGGCAAGTCTTTCCACCCAATCCCACAAAACATAAGCC | | |
| | AATGCTGGGACCCT | | |
| cg17508639 | TTCAACAGTTAGGACAATGTGAACTGTAGCTCAGCTCTGCTAGGTACCTTCACAA | −0.09365931 | Hypomethylated with Age |
| | GACCT[CG]AATGCTGCCCCTTACTATGCCTCGGTTTTCTTATCTATAAAAACGG | | |
| | CATTTTTATCTTGT | | |
| cg17588800 | GAAACTCCCTCTCGCGGGATGATGCCTTTGGAAGTTCAGGGTTTTTCTCTCCACC | 0.34708529 | Hypermethylated with Age |
| | GGACT[CG]TCTGCCCTCGGGGCCAAATCCGCGAAGCGAGGAGGAGCTCCCACCA | | |
| | CACAGCCTGCTGTC | | |
| cg17591832 | GACAAACAAAAGGAAAGGCGGAAACGCAGAAACGCAGAGGTAGCCAGTGGATGAG | −0.03873746 | Hypomethylated with Age |
| | GCATGĮCG]CAGAGCCCACGCTCAAAGCCTGGCGCTGTTGGCGCCCAACCTGGAA | | |
| | ACTACCTTTCCCGT | | |
| cg17796960 | CTGGGGGCCGCCGCCTTTGGCCCTGGCTCCGGGCCCGTGTGGCTGGACGAGGTGG | 0.02189651 | Hypermethylated with Age |
| | GGTGC[CG]GGGCAGCGAGGCGTCCCTGTGGGGCTGCCCTGCGGAGCGGTGGGGA | | |
| | CGCGGAGACCGCGC | | |
| cg17804348 | CTCCGCTCTAGCAACGCGGCCACCATCTCCATCGGCGGCTCAGGGGAACTGCAGC | 0.16593646 | Hypermethylated with Age |
| | GCCAG[CG]GGTCATGGAGGCCGTGCACTTCCGCGTGCGCCACACCATCACCATC | | |
| | CCCAACCGCGGCGG | | |
| cg17921331 | GAACCACGTGCCGGTAGGAGGTGGCCAGGTAGTCGAAGTAGTTGATGTTGAGTTT | 0.01251008 | Hypermethylated with Age |
| | CCGGG[CG]ATGTAACGGCCCAAGTATTCCATTTGCTGTGGGAGCAGGTGGCGCT | | |
| | CAACTGGGGCCGGG | | |
| cg17947364 | AGGGCCTGTGTTGCTCATTTAAATGCAATTTGACGTATGTTTGGGTCAGTGGCCT | 0.03500313 | Hypermethylated with Age |
| | CAGTC[CG]GAGGCTCTTACAGTAACTTGGCACTGTGGAAATCCCAGAACTTCGG | | |
| | CCAGGGTCTGGAGG | | |
| cg17980999 | GTGATGCCGCGTGTCTGTCGAGCACCTGCTGTGCTGGGCACCATCGTAGTTCTAG | 0.03133831 | Hypermethylated with Age |
| | GGCCT[CG]TCAGTGAAGCAGAAAATCCCTGCTCTCGTGGAGCTCACGTGCTGGG | | |
| | GGAGACAAACAGGA | | |
| cg18104354 | GTTTCCTCTTTGCTCTCTCTGCCACGCTTGAACAACTTGCCAAGGATGCCATCTC | 0.05371406 | Hypermethylated with Age |
| | TCTTA[CG]TCCGCCGTGGTGCTCCGGATTCTGACTCATTCGGACGAACGGGTCG | | |
| | GGGTCGCGGAACCT | | |
| cg18192222 | AGAGTGTAGCCGCCAGTCCTTCCCTCATCCGGAACCTTCTGGTCCACATTTCCTT | −0.03276835 | Hypomethylated with Age |
| | CCAAG[CG]TTAGTCCTCGGCTCTCCATGCTTGGCTCCCTAGTGTCACACACCGT | | |
| | CCTTTCTGGGTGGC | | |
| cg18262801 | GGGCCTGTCAGCTCACTGGGCCATGCCTAGTAGATCCACCCTTTCCCATCTACAG | −0.0239576 | Hypomethylated with Age |
| | ATGCT[CG]TACTTACCCAGGTTCCGGGGCACGCGCGGGGCAGCCCCCGCCTTCC | | |
| | AGAGAACGGCGGCT | | |
| cg18369516 | AGGGGCCGGCAGGGGGCGCCCGAGCCCGTCTCTGGAGCACAAAGACCCCCGGCCC | 0.03735523 | Hypermethylated with Age |
| | CTGCG[CG]ACCCGGACGAGCTCGCATTCAGCCAACGACTCCTGTGAGAAACATT | | |
| | CCAGCAAGCACTCG | | |
| cg18376860 | TTGCTCCAGGGGAGCCCCAGAGTTGGTGGCTGGCTAACCCAAGGCCCCAGCGGCA | −0.05008836 | Hypomethylated with Age |
| | GCCTC[CG]CCCGGCCAGCTCGCCATGGCACGGGGTCCACAGACCCTGGTGCAGG | | |
| | TGTGGGTGGGGGGC | | |
| cg18404335 | TGGACTAATGTGACCAGAGTCGGCTGTGTTTGGAAATAAACTTCCAACGCTCCAG | 0.056409 | Hypermethylated with Age |
| | ACTAG[CG]AAGCGTCGTTAAAAAACCGAAGGTACCCTGAGTGGTTTTTAGAAAC | | |
| | TGAAATTCTGCAGC | | |
| cg18448426 | GCATTACAATAAATATACAATAAGCATCCACAAGGCATGTGTGGGAGACTGTGCT | −0.16554624 | Hypomethylated with Age |
| | TATGC[CG]ATGGCACGGGCAGGAGTAAGGCAGGCATCAGGGGAATGTGGATGCA | | |
| | CGGGAGGATGGGGA | | |
| cg18480675 | TAAGCATATTTACAAAATATACAAGGAAAAGGACCTGATTCCCTGGCTGGGTGAG | −0.36115647 | Hypomethylated with Age |
| | TGAAA[CG]TTAATTCTTCACCTTGGCACCTCAACCTACAGCTGTAATAAGGGCC | | |
| | GTTATGCCCAAATA | | |
| cg18515624 | TTAGTGTTCTTCTGACCTGGCCTGTGTAGTCACAGCTCTGCCTGCCTGCCAGCCT | −0.50652021 | Hypomethylated with Age |
| | GGGCC[CG]TGTACTTCCACAGCTGCCCCACAGCAGGGCAGGCTTCTTTCTGCAA | | |
| | ACAGTCTAAAATCG | | |
| cg18557556 | TTTAGCAGGCGGTATAGGCAAATTGGATACTGTTTTATCCGTAGTTTCCTCGTGG | 0.03242952 | Hypermethylated with Age |
| | CCTCA[CG]GCCTAACCTGGACCATCGGACAGCGGAGTGCTACCAACCTGTCATT | | |
| | GATGTCATTCATTC | | |
| cg18567954 | GCGGCCCTACACGGCCACCGTGTGCCACCACATTGAGAACGTGCTGAAGGAGGAC | −0.00279857 | Hypomethylated with Age |
| | GCTCG[CG]GTTCCGTGGTCCTGGGGCAGGTGGACGCCCAGCTTGTGCCCTACAT | | |
| | CATCGACCTGCAGT | | |
| cg18710383 | CCCGCCGGAAGCCGACATCTCGAGTTCTGGCAGAAGCAATTTGCGCGGCGAGGAG | −0.04651437 | Hypomethylated with Age |
| | CGGAG[CG]GCAGGAACCCAATAAGCTGCTTCGCCTCGGAGCTGAAGCCCGTACT | | |
| | CAAGATGGCGGCTC | | |
| cg18776463 | GCGCCTCCCTTACCCAGCCCCTTGCCACCTTTACCTCGCCCAGACATTCTGAAAT | 0.12933125 | Hypermethylated with Age |
| | CACAG[CG]CCTACTCAGCAGAAAATCGGGCTGCCCACTGTGATTGCCAGTCGCA | | |
| | GACCAGGGCTTATA | | |
| cg18781966 | GGTCCCAGATTCCTGCTCTGGGAGGGGCCCTGGAATACAGGCTTCTCCCAGTGCC | −0.17695148 | Hypomethylated with Age |
| | CGAAA[CG]CCCCCTTTTCATCCCTTTTGGAACTGGCTTCCGTGGGGATTCTCCG | | |
| | GGTCCCGGTGCCAA | | |
| cg18792364 | GGACTGAAGCTCCCAGCACAGGGGAGGCGCCCAGCGTGGCTGCAGAGGAAGGGAA | −0.03505887 | Hypomethylated with Age |
| | CGCAC[CG]CAGGTGGGAGGGGCCCAGCTGATTTCTCAGCGTCACAGTGAAAGGC | | |
| | ACCCGTGATGAGAC | | |
| cg18822950 | AAAGGTTGGCTCCACGGTCCCGCCGGCCGCGCAGGTCTGGCTGAACTGCTTGGGG | 0.03905871 | Hypermethylated with Age |
| | TCGCC[CG]GCTCCTCTCGATTTTATGAAAATGGCCTAATTGAGGTGTGCTCTTT | | |
| | TCTTTCCTTCCTCT | | |
| cg18862597 | CCCTCAGGCCGTCTTGTCAGACTCTGAGAGCGGCGTCCAGCTGAGCGGCTCTGAG | 0.07063161 | Hypermethylated with Age |
| | CGCAC[CG]CGGATGCTTCCAACGGCAGCCTGCGGGGGCTCTCGGGCCAGCGGAC | | |
| | CCCGTCCCCACCGC | | |
| cg18898632 | GCGGAGAGAGACGCCGGGGCGGCGATGCGCTTCCCACAGCAGAGACGCGTCAGAG | 0.01113335 | Hypermethylated with Age |
| | TCCAA[CG]GGGAAAGGTGGAAAAAGACGAACCGTGTAAACAATAATCAAAAGGG | | |
| | ACATCGGGTGGCTG | | |
| cg18933331 | ACCTCCTGCTTGGGTTCAGCCACCTTCAAATACTGCATCAATGGCTCGTGCCTCT | −0.46388787 | Hypomethylated with Age |
| | GCCTG[CG]GGGCTGGGCCAGCGCGGGAGAGGCAGGCGGAGGGTTCAGGGAGCTG | | |
| | GGGATCTGCGGTAT | | |
| cg18948877 | AAATGTTTAATTGTAAAGCATGAGTCTGACCTAAAAACAAGTGTGCCCGCAGAAG | 0.06906789 | Hypermethylated with Age |
| | TGAGG[CG]GCACGCCCGTTACTCCTCACGCAGGAAGCGCAGCAATGAAACAAAA | | |
| | CGCCGTGCGTTTAA | | |
| cg19025497 | TTCCAGGCTGGAACTGCGAAGTTTCCTGTCTGATTTTCCAACAATGTAATTTCTT | −0.00096167 | Hypomethylated with Age |
| | TCTAG[CG]GAAGGACCCTCAGAAAGCAATCAGAGGGGTGCGGAGTAAAAATAAA | | |
| | TCAAGTTCTTGTGA | | |
| cg19065773 | GCAGGGGCAGCAAGGGGGGCTTGTGGAAGTGCTGCACCAGCTCCGCGGACAGCAG | −0.00478023 | Hypomethylated with Age |
| | CACCA[CG]ACACAGCGGGTGCTGAGGAAAAGGCTTAGGTCCTCTGCCGAGAAGG | | |
| | AGGCCTCGGGGCCC | | |
| cg19256400 | GGTCACCCAAGGGGGGGAAGGTCTTGGCTGGAGGATGAAGGGGCCTCTTGTCTCT | 0.00253747 | Hypermethylated with Age |
| | GGGG[CG]AGGCGATGTCAAGGGGAATGACAAACCAAACCAGTCCAAAGCAAGGG | | |
| | GACTCTGTGTCCT | | |
| cg19265972 | GCTGAACGTCTCTCTCAGGCCCCGCAGCTCCTCCTGCAGCTGGGAGTCTGGCCAG | −0.15695357 | Hypomethylated with Age |
| | GACAG[CG]TGCAGAGAGAAGAAAACGGGATCGCTGTGTCCGCCACCACTGCACC | | |
| | CACCACTCCCTTGC | | |
| cg19283806 | TCCGTAGTATTGTCTCTGGCTTTGAACGCTGTTGAGGGAGGGGAATGTTTGCACT | −0.32000571 | Hypomethylated with Age |
| | CATCC[CG]CATCCTTTTTTGGCTGCTATCTTTGCGGGGATTGTTCAAGGAGAAA | | |
| | TCCATCCTGACTGG | | |
| cg19513321 | AGGTGTCATCCGAATTCAGGCTCCTGGGGCCCGGGAGGGTCCGACTCTACGGACC | −0.05784855 | Hypomethylated with Age |
| | CAGGT[CG]CTGTGGCCCATCGCTTTCGATTTGACTTGGTTTCTGTCGCCACTCG | | |
| | CGGAAGGCGCGCCC | | |
| cg19539667 | GCCGCCCTTTTCGTGGTCCCAGGGCCCTTCCAAGAACCGGCTAAACCAACCCAAG | −0.02688503 | Hypomethylated with Age |
| | CCGCG[CG]CACACACACTTGTGCACACAGGGAGTGTGGAGCCAGATTCGATAAG | | |
| | GCACCCCGTGTGCC | | |
| cg19595402 | ACGTCCCCCACCCCTGGATGGCTCTCGGTGCCGCGGAGCGGGCCCCCATCTCCGT | 0.11810405 | Hypermethylated with Age |
| | GTCCC[CG]CCCCCCGCCCAACCCGAGGCGGCGATCCCGGCCCCCACAGTCGCTC | | |
| | CCCCTTACCTGCGG | | |
| cg19600115 | ATCCCGAGGTCAGTCCCTCCCATTCTGGGGCACCCCTGGAGCAGCTAGCTCCCTG | −0.04430982 | Hypomethylated with Age |
| | CCTAG[CG]GTGGCAAACTCCAGAGCCCCAAGGAGCAGAACGGGGATTCCCTGTG | | |
| | CGCAGGCTCCTGGT | | |
| cg19668234 | CCGCATGCCGCGGCCTCTGGTGAGCTGGGTGGGGATGCTCCTAGTGCCCCGCCTG | −0.04462914 | Hypomethylated with Age |
| | CGTGG[CG]CCCCCCAGGGGCCACCCGCCACGGCGCTCGTGGTGTCTGACCCGCA | | |
| | AGGGCGCCCCTAGT | | |
| cg19706682 | ATAACAATAATAATAATGGTAGCAAGCAACGCTCTGCAGTAGGGGCTTCTCTCGC | −0.00063053 | Hypomethylated with Age |
| | CATTT[CG]TACTGAGGAGGAAACATACTTAAGAGGTTACAAAACTTGCACCAAA | | |
| | CAGATAACCCTCGG | | |
| cg19753794 | CTCGGGCTCCGTCAGGCCGGCCAGCCGCGTCCCCGGCAGGGTGCGCAGCGTCGAG | −1.01886053 | Hypomethylated with Age |
| | CGGTA[CG]TCTCATGGCGCACGCCGCCCACGTTGATCACGATCTTGCCGCTGTC | | |
| | GCCACCGCCGCCGC | | |
| cg19761273 | GGACAAAGCCACCACCTTTCACAAAATGAGGCCAGACCACCTGCCTCCCTCCAGT | −0.16006978 | Hypomethylated with Age |
| | CCCTG[CG]GCCTGGAGACGGAGTCAACATTCTTATCTGTGTTGGATCTGAATGT | | |
| | TCCTCCTTGCAAAG | | |
| cg19772907 | CTGGCCGCCTAATAAAAGCTCATCCCCGATTGGCTGCCCCGGCAAATCGGAGTGT | 0.55113305 | Hypermethylated with Age |
| | AAAGC[CG]CCCCGGATTGGCTGAAACACTTCCTGAGCGATTATCTTTGTGAGGC | | |
| | TCGGGTGAGCAAGA | | |
| cg19803194 | CATGTTGTAGGTGGCCCTGGTCCCCATGATCCATGGAACAGAGGCGGCCCCAGCT | 0.070962 | Hypermethylated with Age |
| | GTCTC[CG]CAGGATTATGCACCGCGCGTCATGAGCCGAGGGGGACAGGACCCGT | | |
| | GGAGCACCACTCTC | | |
| cg19848940 | GAAAAAGTAGAGAAATGTAATATTTCTTTCCTGCTGTACTCACTGTAACTGTGAG | −0.12096991 | Hypomethylated with Age |
| | AGGAT[CG]GCTCTTTTAACCAACAGATAAGAAAGGAAATATTAGCTATGAAGAA | | |
| | ATGTCTATCAAGTT | | |
| cg19968421 | GGGGACTTGGCAATGCCAAGGTGTTTGCTGAGGCTGTGGACTCTCCAGCCCGGGA | 0.01000635 | Hypermethylated with Age |
| | GAGGT[CG]CCGGACCTTTGAGGGGCATTGGAATCCTGGGCTCCTCCTCTGCTGG | | |
| | GTGGAGCCGCGAAC | | |
| cg19996355 | AGCACCTGGCCTGTGCCTCGTCCAGGCTCTGGTCGGTGATGGCCATGATCTGCTG | 0.79961665 | Hypermethylated with Age |
| | CAGGA[CG]TCGCTCGTGTCGAGGCGCCGCGGGGGGGGGGGGGATGGCGCGGGGC | | |
| | GCGGCGGGGCGGCC | | |
| cg20143982 | CCCCTGAGGCCTCCACCTCTGAAATCTGCAGAACAGCCAGGAAACAAGCCCGGCC | −0.45561767 | Hypomethylated with Age |
| | CATGG[CG]CCACCTGCTGCCTGCCTGCCTCCGCTCATGCACCCTGGGCTGGGAT | | |
| | GGTACTTCTGTTCG | | |
| cg20160695 | GCCAGGCGCAGGCGGTCCTCACCCAGCTTCAATGCGCTGGAGGCTTGGCAGAGCT | 0.30280796 | Hypermethylated with Age |
| | GGTCGĮCG]AGGAAGTACAAGCAGGCTGCCAAATGCTTCCTGCTGGCTTCGTTTG | | |
| | ATCACTGTGACGTC | | |
| cg20210376 | GCTGGGGATGTGACCGGTAGCCGGGGTTGCAGTGGCAGGAGTAGTCAGGGGGGCC | 0.00594985 | Hypermethylated with Age |
| | CGGCA[CG]CACTCTCCGTGGCCACAGATGTTCTGGTTCAGTCGGCACTCATCAG | | |
| | TCTCTGCGGGCATG | | |
| cg20233029 | GGAGGTGGGACCAAGGCCGAATTTAGGGACCCCCAGCATGAGCTGCGGGTGGGTC | 0.00210136 | Hypermethylated with Age |
| | AAGGC[CG]CGAGGTTGGAGGGCACGGGGACACAGCATAGCGAGGAAGGAACGGT | | |
| | AGGGACCGTGGTGA | | |
| cg20278383 | ACCACCAAAGCGTTCTGACCGGACAGTGTCACTGGAGAAGGCGGCGCGACATGTC | 0.00226832 | Hypermethylated with Age |
| | CAGGG[CG]CAGATCTGGGCTCTGGTGTCTGGTGTCGGAGGGTTTGGAGCTCTCG | | |
| | TTGCTGCTACCACG | | |
| cg20322193 | CTAAACCGACTCCTGTATTAAAATAATACACTATTATTGTGAAGTATTATCTGAC | 0.01347078 | Hypermethylated with Age |
| | AACAC[CG]TTTTTTGACCTTGGATTTCTTTAGGGCAGGAGCCATGTCCTACTTA | | |
| | TGACTGTTTCTATG | | |
| cg20370909 | AGAAGGCGCTGAGCTCCAGCCACGAGGAGAATCTGAGGCGCACACGATGGTGTGA | 0.06419686 | Hypermethylated with Age |
| | GGCGG[CG]CGGGAGGGGCCTGGGTCAGAGCTTTGTACAAAACAGTGTTTGCGGA | | |
| | ATGACAACAGCCAC | | |
| cg20386580 | CGACCTGCCTTTCAGCCCTGAGGCCCTGGTGGACCGCAAGGAATTCTGGGCCGTG | 0.17597683 | Hypermethylated with Age |
| | TGCCG[CG]TGCCCGGGCCCCTGCACAGCGGCGACATCCTGGGCCTGGTGGTCAA | | |
| | CGCCGACGGCGAGC | | |
| cg20404336 | ACCGTTGAGCCATTGGTGTCAAGTATTTTAATTCTCTTTAAAATTTAAAACCTGC | 0.29049971 | Hypermethylated with Age |
| | AAGCG[CG]GGAGCTCAGGGACCTGGCCAGGAAGGCCTGAGCTTCCGGGTCATCT | | |
| | TAGCACGCCCCCTC | | |
| cg20417869 | AGAGAAGCAGGAAGGAGGGAGAGGAAAGAGAAGAGGGAGATGGACTGGCCTCAGC | 0.09414194 | Hypermethylated with Age |
| | CACCC[CG]GAGTACAGGGATGTCATCACACCAGCCCTCCAGCGGCTGAAAGAGC | | |
| | CAGTGAGAGGCAGG | | |
| cg20419623 | CCCCACCACCCCCCCGGAGTACTTAAGGGAGTTGGCGGCGCTGCTGCATTCATTG | −0.03798525 | Hypomethylated with Age |
| | CGCCG[CG]GCACGGCCTAGCGAGTGGTTCTTCTGCGCTACTGCTGCGCGAATCG | | |
| | GCGACCCCAGTGCC | | |
| cg20697767 | CCAGCCACGTGCCCAGCCCGTGGGTGCGGTTTCCCAGGGCGGCCCCGGCGCCGCC | −0.0376877 | Hypomethylated with Age |
| | GCCCA[CG]CCGTCCACCTCCTCCAAGCCGCTGTGGTTCAGGAAGGTCGCGTTCA | | |
| | TCGCCGCGCGGCGC | | |
| cg20744625 | TGAGTCCGAAAGAACGGGGGAAAGCCGAATTATGCAAATGCGGATCCCTGCGATG | 0.21122692 | Hypermethylated with Age |
| | GGGCT[CG]GGTTACGGCCCCCGCCGGCCCCTTAGGTGAGGCACCCACCGGCAGC | | |
| | AAGCGCGGGCGAGG | | |
| cg20782850 | GCATGGCCACCGCAAGCGCAACTAAAATCCAGGGCTTGTCGCAGGCACAGGCTCC | −0.21007724 | Hypomethylated with Age |
| | TCCTT[CG]GTGGGTGGGACGGCGGCGCGCACTTTCTCTACGCCCCACTGCTAGG | | |
| | ATGTGCGGCCACCA | | |
| cg20831777 | CTGTGAGGAGGGTAGTGGGGGGGCATTGATGTTCCCGTTTCTGAGTGAGGAGACC | −0.21684005 | Hypomethylated with Age |
| | TATGT[CG]GGGCCTTGGTGACCTCAAGCCGAATGCTCAAACTGCTACTGCTACT | | |
| | CCTAAGGTTGTGCC | | |
| cg20912517 | TGACAGCCGCAGACACGGCGGCTCAGATCACCCAGCTCAAGTGGGAGGCGGCCCG | −0.05364615 | Hypomethylated with Age |
| | GGGGG[CG]GAGGTTCATCCTCACAGGGATAGGCACCTATTAGATGTGGTGTGGT | | |
| | TTTCCTCTCTACTC | | |
| cg20916483 | TCACGGAACGCCGAACCTGGCCGGGCCCGGTTGCTGCTGCGGTGTTAGGTGAGTT | −0.02140525 | Hypomethylated with Age |
| | CAGGC[CG]CCGCCATCGCTTCTCAAGCGCACCAGCCGCGCCCCGCCCCGCGCCA | | |
| | CACGCAGCCCGCGC | | |
| cg20939114 | TAAATCCAACGTGACGCCTACCGAGCTGGGTACAACATTGGTTTGTTTCATAAAA | −0.01195967 | Hypomethylated with Age |
| | CAGCC[CG]GGGTGTTTGGGACTCTTACTCCTTTACAACTCTCATTTGACTGTCC | | |
| | AAAGATGCTTGCAC | | |
| cg21010407 | GCCGTGAGAACCCGGGGACAGCCTCCCCTCTTGCGGTCCTGCAGTCCCGGACCCA | −0.19818736 | Hypomethylated with Age |
| | CTGGG[CG]GATCAGAAAGTTTGCAGGGAGCCAGGGACTAGGAGACAGACAGACA | | |
| | GCGCAGGGACAGAG | | |
| cg21160852 | CCTCACCCTCGTAGGACGAGCGCCACTCGGCCTTGTGCTTCTTGTGCTTCTTGCC | −0.07214754 | Hypomethylated with Age |
| | CATGG[CG]GCGCCGGCGGCGGGCCCGAGGCGGGGGCTGGGAACAGCTGGCACCC | | |
| | GGTCGGACCTTGGC | | |
| cg21165519 | GGTGGCGCGCATTCGCCCACTCGGAGACCGAGAGGCAGGTTTTCTGCCTGCACAG | −0.22639149 | Hypomethylated with Age |
| | CCTCC[CG]CCCAAGGCCAGACCTGCTGGAGGCCCAGGCCCTGGAGATGGCTGTC | | |
| | TTCAGGGAGACTCT | | |
| cg21200656 | GTCGTGTGCTTTGGGCTCAACGACATGGCTCGGGGGGCAGCCAGGTAGGGGGGCC | 0.04698382 | Hypermethylated with Age |
| | TGGGG[CG]CGCGCCGGCAGGACGCGGCGGCCGCTCGTCGCCGCCACCTCGGCCG | | |
| | CGGCAGCTGAGCCT | | |
| cg21213853 | GTCCTCTGGTCCTTCTTTCTGTCTGTGCCTCCGTCTTTGTCTCAACCTCTCAGGC | 0.19922508 | Hypermethylated with Age |
| | TTGCT[CG]CTCCCTGCCCAGATTTTGTGGCCCAGGCTCCTGGCTGTCTGACTCC | | |
| | GGGTTTCTGTCCCC | | |
| cg21434114 | GAGGGGAGGGGAGAGAGTTGGGCGAGGGAGAGCCCCCGGCCGGCTGCCAGAAGAT | −0.0701061 | Hypomethylated with Age |
| | CCCGG[CG]GGAGGAAGCCCAAGTGTCACTTGAATTCCACCCAAGGAGCGGGCGC | | |
| | CTGGGATCAGAGCG | | |
| cg21514227 | TGGAAAGAGAGGCAGGGCCCACGGACAAACAGACTGGGATGGATGCATAGACAGA | −0.00640256 | Hypomethylated with Age |
| | CGGAT[CG]ATCGGGTGGATGGGCTCACTTGCAAGTGCGCTCGCGGCCACCGGCG | | |
| | TGGCAGTCGAAGTT | | |
| cg21529788 | GGAGCCAGGGGCACCGGCGGAGACACGAGCGCAGAAGGCACGCGCTCAAGCCCAC | −0.16576542 | Hypomethylated with Age |
| | GCCCG[CG]ACTGCCGGGACTGAAGGTGTTGCGAGCCCCGGCTCCACCCCTAGCC | | |
| | TGGGGTGCGCCGTG | | |
| cg21596317 | TGTAACGTGTTAGTCATCCATTATCTGCTTCTATTTTTGCAGGTTCTGAATGATG | 0.2114108 | Hypermethylated with Age |
| | ACTGA[CG]CGGGTTTGGGTGATACCCCTCACAGCCCCTGTCATTCCGGAGTCAT | | |
| | AAGGCACCCGCGCG | | |
| cg21596498 | CTAGGAGTGCATAGGCAAGAATGTCTGCTGCTCACAGAGATGGTGGCCTGGCTGG | 0.00283019 | Hypermethylated with Age |
| | GTCTG[CG]CTAGGCTGCCCTGTTCTGCCTTCTATCCTCTAGTCTTTGTTCCTTC | | |
| | AACTGATATTTCCT | | |
| cg21649277 | CTCCAAAGACCCATCCTCCCAGGCAGCCTTCCAGGCTGATCACTGTGCCTCCAAC | 0.01680472 | Hypermethylated with Age |
| | TCCGT[CG]TTCCTGTTCCGATCCCCATCACGGGCTGAGGGTGACCGTGTTTGTT | | |
| | TATGACCGCCCCCT | | |
| cg21868031 | GTCTAGACTCTGGATCTCTATTTTTAGGTCGAAGTGCTTTATTCTTGACTCCCGA | −0.03669604 | Hypomethylated with Age |
| | ATTCC[CG]GAAACTATTACCAAAGCAGCTTAGTTTTCTCTCCACCCTGCCTGGG | | |
| | TCACAAATATGACG | | |
| cg22025854 | CTCAGATCCAAGATTCTGGGTACCCAGGCCTTCTTCCTCTACCACTCAGAAGTGG | −0.11214444 | Hypomethylated with Age |
| | GGTAC[CG]AGGCTTCTTCTCCCTTAGGGACCCAAGACTCCTGGCCTCAGGCCCT | | |
| | CCTCCCTGAGACCC | | |
| cg22249752 | GGCGGCCCAGCAGGGCAGGGCCGGGCACCTGGCAGGTGGGGTCATCCCCACAGGG | −0.05509014 | Hypomethylated with Age |
| | CACCC[CG]GCCGAGGGCAGAGCTGGTTCCGCCGCAGGGTCGATCCTGGGCTCTG | | |
| | GGCTGTGCACACGT | | |
| cg22341865 | GCTCCACTGATCCGGCCCCCGGGGGTGGGCGGGCACGTTTGCCTCGGATGGTCTA | −1.08906271 | Hypomethylated with Age |
| | ACAGG[CG]TCGGGGAGAGCCAATGGTGTGGCTTCATGGCTCCACCCCCTTCCAT | | |
| | TCGATTGGCCGGCG | | |
| cg22353329 | GGCCCTCCGGACTGACGCGGCCTGAGCAGCAGCGAGTGTGAAGTTTGGCACCTCC | 0.36581392 | Hypermethylated with Age |
| | GGCGG[CG]AGACGGCGCGTTCTGGCGCGCGGCTCCTGCGTCCGGCTGGTGGAGC | | |
| | TGCTGCGCCCTATG | | |
| cg22514963 | AAAAACAAACTAGCTATGGAAGCAGGAAGTGAGGTCAGGCTGGAGTTGCAGGGAC | −0.02361098 | Hypomethylated with Age |
| | TGTCC[CG]GGTGGGTTACAAAAGCAGGACCTGCTTAGACTATAGACAGGTGTGT | | |
| | GTGTGTGTGTGTGT | | |
| cg22584681 | GACCGACTGTCAAGGTTTATGGTCGTTGAGGGAATGCCGTGGGAATCAGTGGTTG | 0.05234669 | Hypermethylated with Age |
| | AGAAA[CG]GGGTAGGAAAGTGTGTATCAGTGAATGAACTTGGTGTCCATTCCGG | | |
| | GTTTGCAATTTAAT | | |
| cg22796704 | TCCTAAGCCTCTCTGAGCTGGGCTTGGCCACCTTCCGGGGTGTGAGCGTCCACGG | −0.23229928 | Hypomethylated with Age |
| | GAGAT[CG]ACCACACCAGGCACCCAGGAGCAAGTGCTTTGAAATGCGGCTTTCT | | |
| | CCGGACCTTGCAGG | | |
| cg22901840 | GTGCAGGGAAAGCACACCGTGGCTGCAGCCCAGCAACTGGCAGTAGGTATTTTCA | 0.14302156 | Hypermethylated with Age |
| | ATGGT[CG]GCAGGTACTCATGACGGAAGTTGCCGCTCGCCCACTTGTGCAGCAG | | |
| | CGTACTTTTCCCCA | | |
| cg22943590 | TAAGACCACTTGCTGCTCCCTGGAATGATTCTAATATAGGAGGTACATTAGAGAG | −0.26000291 | Hypomethylated with Age |
| | AGTGC[CG]TAAGAATAGCCTATATTAAAAAGAACTAGGTATGTAGCTTTTAAAG | | |
| | TGTGCCCATTTAGA | | |
| cg23027329 | GGGCTGGAGGCAGGAGGGATGCTCCCTGGCAACCTCACAGGGTGCCGTCACATGA | 0.00236454 | Hypomethylated with Age |
| | CAGGG[CG]GACACATGACAGAATTATAATAAAATGCTTAAGAAGAGTAAGCAAC | | |
| | AGCATCTTCAGATT | | |
| cg23040782 | TCCCTAGGGCTTTTCCTGTACTAGAAAGGGAAACTTTGCTTTTTTTTTTTTTTTT | 0.58265094 | Hypermethylated with Age |
| | TGAGA[CG]GAGTCATACTCTGTCGCCCAGCCTGGTGTGTGTGCAGTGGCGCAAT | | |
| | CCCGGCTCACTGAA | | |
| cg23078123 | AAATGCCTTGGCCACAAGGAGAAAACATTCGCTGGCTGTGGCTTGAGCCAACTGG | −0.09788592 | Hypomethylated with Age |
| | GTCAG[CG]CCAGCCTGTGCAGATGTCTTTGTGGACAGAGAAGAAGTCCCTCTGG | | |
| | GAGAAGGCAGCCCA | | |
| cg23083277 | CCTCGAGTAGACAGGTGGATGTGAGAAGGATTCTCCTTTCTGGTGATTTGCACCT | 0.04493303 | Hypermethylated with Age |
| | GTTGA[CG]CCCCCAAATCTGAGCTCTAAGGAATTTTACAGATGGTTAAGACAGG | | |
| | TTCTGAAAGGTAGA | | |
| cg23115907 | TGCCCCACTGACAGTCACGCTGCGAAGGCAGCTCTGGCCAATACCAGAAGGATGG | −0.07572133 | Hypomethylated with Age |
| | GCAAA[CG]CAACATTGGCATGGATGCTTTTGCTACACCGAATTTTGTTTTTCTG | | |
| | TAATGGGGGAAAAA | | |
| cg23156348 | TGGGCCATTGGTCAGTCTAGCCTGAGGGGGGGTTGTTGGGCGGAAGAGAGAGACT | 0.994192 | Hypermethylated with Age |
| | TCTTC[CG]GCCTCACTCGCTGTCACCATAGAGATTGCCCATCCAGGCAGCGAAG | | |
| | CAGCAGGGCCAGGC | | |
| cg23195200 | ACAGGACAAGCTCTGAATTGCATTCCAGGAAAAGGGGACTGAGGCTTTGTATTTT | 0.06724461 | Hypermethylated with Age |
| | TCCTC[CG]CACCAAGATTCCCAAGGCTGCTGTTAAGGGTTTTTACCCAGGGTGG | | |
| | GGTCCACGGCGAAG | | |
| cg23235965 | TTTCAAGCCCTAGGTAAAAGTGTGTCCTGCCTCGTTACTGGGAAGCACCATCCAC | 0.15332756 | Hypermethylated with Age |
| | ACACA[CG]AGCCTACCCAGCCTGGGGCCCTGTGTGCCAGCACCTACTCTTTTTT | | |
| | TTTGAGACGGAGTC | | |
| cg23299919 | CTCTTCCGATAGTTCTGGCAGAGACGGCTGGGGGGAGACACGGGCGGAGGCGGGT | −0.05181833 | Hypomethylated with Age |
| | CCGGG[CG]GGCCCCACGTCGCGCAGCCTCAGCGTGTTGCGCCTCCCGCGGCTGC | | |
| | GGCCAGAGACTTCC | | |
| cg23480021 | TGGCACGACAACCCAGAGGGAGGAAGACCTTTCCAGTAGGTTTTAGAAAACATCG | −0.01736771 | Hypomethylated with Age |
| | TGAAC[CG]GAATTCAGTGGTCACCTGAAAGGCACATTTCACAGACCAGCTAGCA | | |
| | AACAGACTCAGCAG | | |
| cg23491424 | TTCACTTCGTATTTTTAGTTCCCAAACCTTGACATCCAAGCTCCCGCTCTGTGGC | −0.11805419 | Hypomethylated with Age |
| | TTCCG[CG]GCAGCCTGGCAAGCTTCCTGTGAAGTCGCCCGCTGCTGAGCTTCCG | | |
| | CACCCTTCCTCCCG | | |
| cg23538901 | AGGTCAGATACTGTCTGCCTGATGTAGCAATTCTCTCAGGCTGTATCTGCGATAC | 0.07953068 | Hypermethylated with Age |
| | CAGTG[CG]GCCGGTGAAGAGGGCGATGGTGAATTGCACCAATAATGCTGTAGGT | | |
| | GGCGCTGTGTACTT | | |
| cg23588049 | GGAGGGGGGACAAGGCTTGCTTGCGTCCTCCGTAGATTGGCAGGTCACTGGGACG | −0.01746583 | Hypomethylated with Age |
| | GCCAG[CG]CGTGCGCACTGGCCTGTCAGCGGCCGGTGGACCATGGAGGCCGCAA | | |
| | GGCCCTTCGCCCGG | | |
| cg23652182 | CAGGGGGTCCCGGGGACCCACGGGGTGGGTGGGGGCTGCGCTCACCTTGGCCTCC | 0.05379296 | Hypermethylated with Age |
| | TGCAC[CG]CCTCGTCCAGCGGCAGCACGGCGTGCTCGCGGTGCTCGCGGGCGCG | | |
| | GTCGCACACCACGC | | |
| cg23684204 | ACTCCCATATGCTGGGCCTTCAGTACGTTTGTCTTTATATTGGGAGTCCTCTGTG | 0.08640732 | Hypermethylated with Age |
| | GCCTC[CG]CTGAGGGCGCTGGGGCGGGGGTTGGGGTTGGTGGCCAGCTGAGCCT | | |
| | GGTTGAAGGGCGGC | | |
| cg23757489 | CAACAGGGACAGAAGGCAGGTCCCAGAAAGCAGGTTCCCCCAAAACTGGCTTCCC | 0.00124377 | Hypermethylated with Age |
| | TAGCA[CG]GAGTTAAGGCTGCAGCCGGCTGCCTAGAGAGAAGGGTGGGCAGGGA | | |
| | GACAACGCGGTGAG | | |
| cg23832822 | CTGACTCTCAGGAGGACCAAGCATTCTTGGGGGAGACAATTTAAACTACCAAGGT | −0.09451466 | Hypomethylated with Age |
| | ACACC[CG]CAACTCCCTCTCGGAGTGCCCTTACGTTCCTTCGGCTGGCCCCTCG | | |
| | CCCCAAAGGAGCCC | | |
| cg23854009 | TTCCCCTCCACCTTTCTCCATAGCAACGGGGTCATTCCTCCCTGAGGTCCAGGAG | 0.79611315 | Hypermethylated with Age |
| | AAGGG[CG]ACCTCTGCCAGCCCAAGAAGCGGTGAGAACTACATTACCCAGAGGC | | |
| | CCGTGAGCCAGGTT | | |
| cg23910392 | TAATATACGTGATAACTTACACAATGACGTTCAACAAATACTTGTGGAGTGAAAG | −0.02900844 | Hypomethylated with Age |
| | ATGCG[CG]ATTCATTGACAGATCTCAGGGATTTCAGAAAGCTGTATAAGGACCA | | |
| | GCTCTTCAGGCTCC | | |
| cg23980859 | AGCAGTGATTAGCCATGTAAACTCACTTTCTGCAGTTTTACCAGCATCTACCTCT | 0.03893805 | Hypermethylated with Age |
| | GAATTTACCTGTGAAAGTGGATAAGTCCCCTGTAGCGTGCTTGCACAGATGGCCA | | |
| | TACCA[CG]GATGC | | |
| cg24055029 | CCCGTGGACCTCCACGTGGTAGGTGGTGCCGGGCCTGAGGTCGGGCAGGCTGACG | 0.07680376 | Hypermethylated with Age |
| | GTGCG[CG]TGGTGCCCGGCACAGTCAGCTCACCGCCGGGGCCCTCTGCAGGGGG | | |
| | CTGAGGCCGCCAGC | | |
| cg24065957 | TGTTTTCAGTGTGGTCTCAGAGTGAAGGTTTCTATACAGGGTTTAGTCTGAGAAA | 0.04869741 | Hypermethylated with Age |
| | CAACT[CG]GGAAACAGATGAAAGGCATTTCAGATGATTACCCTAGAGACACAGT | | |
| | GGTTCTCAAGACTT | | |
| cg24114899 | TATGCTGGAACAGCACATTAAATGGATCCCCTTGGTCAGAGGTCCCAGAGGGGGC | 0.07065254 | Hypermethylated with Age |
| | CCAGG[CG]ACTTCCTCTCAAAGGACATGAGTAGTCAGACCTGCCTTCCATTTTT | | |
| | TTCTGCCCTTACCT | | |
| cg24119085 | CCATGGCGAAGTGGCGGAGGTGAGCACCTAGAGGCGACCCTGCCCGGGGAACAGC | −0.02048493 | Hypomethylated with Age |
| | TGGCG[CG]ACCGCGGACAGAGCTTCCCACCACGCCCTTCCCCGCCTTTGGCCAG | | |
| | CCTTTGCCGTATGT | | |
| cg24280439 | TCAGGGTCAGAGTGAGCAGAAGCCACTCATCCTGACCGAAGATGACGACGAAGAT | −0.08070786 | Hypomethylated with Age |
| | GACGA[CG]TCCCAGAGGGGGTGGAGCGTGTGATAGGTGCGTGGGGTCTAAGCGG | | |
| | CGGCCTCTGCTCTT | | |
| cg24350475 | GCTGTGTGCGTCTGAGCTTCGGAGGCAGCAGGGTCTGAACATTTTGGACTCAATG | −0.19301333 | Hypomethylated with Age |
| | CTGCA[CG]ACTCGGCCCTCCCAGCTTTGCACCAGGGAGCAGCTGCGAACTTTGG | | |
| | GGCTCCTCGGAGAA | | |
| cg24350628 | TTGACCCAGGGCCAGGGTCTCTCCGCCTTTGCGGGGAAGGGGTGGGGAAGTGTGT | −0.00575263 | Hypomethylated with Age |
| | AGGAG[CG]TGGGTAATTTGGGTAGGAAAAGGCAGGGAGCACCCTACCAGCCCTC | | |
| | CTAGGATACAGGCT | | |
| cg24403268 | TCTCCCCAGACTCCAGACTCTAGAGGGCGACCTCCTCCTGCTCCTGCTCCTGGAG | −0.00397007 | Hypomethylated with Age |
| | CGCAG[CG]AGCGCACAGCGTTTCCGCAGGAATCCTGAGAATGGCAAGGCCCCCA | | |
| | TACCCGCGGTGGTT | | |
| cg24515368 | GGTCTTGTCAGCTCTCTTGATATATAATGTACTAATTCAAGCGGTTCAATTCCTT | −0.00063805 | Hypomethylated with Age |
| | GTTGA[CG]ATAATGATACAATACATGTATTGAGTACTTAAAAGCCACTGACGTT | | |
| | CTGTCTGCAGGGGG | | |
| cg24617313 | TAGAACAGCAGGACCTGCGAAACTCTGAGGCCGCTTTGTGAGGTCCTCCTCTGCG | 0.0148718 | Hypermethylated with Age |
| | CAGCA[CG]CCCCCCACCCCTCTCTTGGTGCCGCCGCAGCTACTCCCTAGGGGGC | | |
| | TTTGCTCTTGGTGG | | |
| cg24731111 | GAACCTAAATCCAGTGATGGAATCTTATTGTCCACAGTCTTTTGTGGTATGTGCT | −0.47842368 | Hypomethylated with Age |
| | ACCAC[CG]AAGATCCTCTCCTAACCTATCATGTGTTCAATACGTCTCAGCTCCT | | |
| | AGACTTGCTGAAGC | | |
| cg24817430 | AAGGCGGCGGCGGCGGCGAACCAGCAGAAGGGACTTTCCTGGCAGCCCGGCGACG | −0.08025663 | Hypomethylated with Age |
| | AGGAG[CG]CGGACAGTGAGTTTGCTCTGCCCCGGTTCATGGTTCCTGCAAGCCC | | |
| | TCTAGGAGGCCGAA | | |
| cg24834889 | GAGAAACTTGTCAACGTCACTTGGGCATCTTAAGAGTGGGTTCGTAAACTTGGTT | 0.14078466 | Hypermethylated with Age |
| | GTGTG[CG]CTGTGCAGATGTCAGTCACCCTGTGTGGTGGGCAAAGCCGACTTCT | | |
| | CCGCCTCTGTAGCT | | |
| cg24852442 | CCCCTCCCCTCTGTGCCCACAGGTGTCCTCCCTGGGCAGCGGCAGTGACCACGTC | 0.00964421 | Hypermethylated with Age |
| | ATGGA[CG]CCATCTCCCAGTGCGAGCAGTACGCCAAGGAGCAGGGCGCCCAGGA | | |
| | GCGCAACGCCCCCT | | |
| cg24860938 | AGCCCCATTTAAGCAGTGCCTCGGGATTTGAGTTGCCTGTGAAACGCAGCAGCCT | −0.23198461 | Hypomethylated with Age |
| | TCTGC[CG]AGGACGCAGAAACCCCCGCCTCTCATCCAGGGCTGACAGGGCACGG | | |
| | GCCGCGAGCCGCGG | | |
| cg24862787 | ATAAGAGAATAAAAGCAGGGTGCCTCAGGTAGAAGTGGCAATCTGCTCGGTCGTC | 0.01486347 | Hypermethylated with Age |
| | TTCCA[CG]CTGTGGATGCTTTGTTCTTTCACTCTTTGCAATAAATGTTGCTGCT | | |
| | GCTCACTCTTTGGG | | |
| cg24900425 | ATGTTAGATTCACCCCACAGAGATAGCGGCAGAGCTGGCAGCGGACGGTCTTTGC | 0.03116228 | Hypermethylated with Age |
| | ATTGC[CG]CCTCCCCAGGGGGGGGGAAGCTGGTAAGGAAGCAGCCTGGGTTAGC | | |
| | TAGGGGTGGGGTCA | | |
| cg24939380 | TTTCTCCCAGCAAGTTTACTTGTAAGCATCTCCATATACTGTTGTAAATAATGAT | −0.17565617 | Hypomethylated with Age |
| | GGTCA[CG]TAGTATCAGTGGCTGTATGCTGAACTCTCTGCACTGACCTACCGGC | | |
| | AGACAGCAACCTGA | | |
| cg24998197 | GCCATGTGTAGGGGAGAGGGAGGTTCCTGGGTTGGCCACAGCCCCCATGGTGGTA | 0.12815987 | Hypermethylated with Age |
| | CAGAA[CG]CATGCAGGGCGGTTGCCCTGTCCTTTGAAGACTGCACACTGTGTGT | | |
| | CAGGCAGCCCTCTG | | |
| cg25067197 | CCAGGTGGCTGCCCTTCCAAGTCCCACACTCTTGTCCTGATGGCCTCCGACCCCG | −0.05879624 | Hypomethylated with Age |
| | GGCCT[CG]AGCCCAACCAAAGGCACCGAAGGAGAGAAAAGCCAACTCACTAGGG | | |
| | TGCCCTCTCCAGCC | | |
| cg25135004 | TCGAGCCACGCTCCACTTCCCGGGAAGAATTCTGGGGAGAGTGAAGGCTCGCTCT | −0.08454937 | Hypomethylated with Age |
| | GTGGC[CG]CCCCGCCCACAGCCCGCCTGGGGAAGCCGCGGGCCCCAGCTCACCT | | |
| | GCAGGTTGCTCACG | | |
| cg25142327 | GGCTCTGCACCAAGGAAGGCTGAACCGAGAGAACCTTGGACTAGAGGTCGTGGTA | 0.20481027 | Hypermethylated with Age |
| | CTCAG[CG]CTCCCTTCACTGACCATTTAAATGTAAAGCAATGTTTGTCCTCGCT | | |
| | GTCAGTCGCAACAC | | |
| cg25150440 | AAAGCATAGTGGATTTAGTCTAACATTTTCCCACCTGGAAAAACTGGGCTTAAGT | 0.00258241 | Hypermethylated with Age |
| | ATCTC[CG]ACAAACAAAAAGCAATACTGCGTTCTTCCCAATATGCCATGTCACC | | |
| | TTTGTAGCACTCAC | | |
| cg25216704 | CAGACGCCGCATGGAATGCGGGGCCGGCTCCACTCCTTCCGTAATGTGGTTTTTT | 0.01495102 | Hypermethylated with Age |
| | ATAGT[CG]GGGGACTCATTGTCCCGTGGCCACTGCCAGCTGTCTGTAAGCTCAG | | |
| | GATTAGAGAGCCTG | | |
| cg25267487 | GTTTCACTGCGTCTTCACCACAAGGACAGCCACTGGCTCCGTCTTTAGATGGGAA | 0.02944397 | Hypermethylated with Age |
| | AACTG[CG]GCCCGGCGACTTTTGCTAGTTGGAAAGGTGGGTCAGTGGTGGAATT | | |
| | AAGTCACACTTAGG | | |
| cg25515801 | GGAATGGGGGGCGGGTGTCTTGCCACTTGTGTGACAGGCTTAACCTTTTTGTATG | 0.28345984 | Hypermethylated with Age |
| | AAGTT[CG]TTTGCCTTATCGGCCTTACTGTTTGATAGTTTACTGTGTCTGATTT | | |
| | CTTCCCCCGTACTT | | |
| cg25553110 | TGGCTGACAGCTGCCTCCAAACACCCAGAGGAGCACATCAGAATCTGGTTTGCCA | 0.13645835 | Hypermethylated with Age |
| | CCCAG[CG]CTTAAAGCATGGCATCAGCTGGTCCCCAGAAGAGGTGGAGGAGGCC | | |
| | CGGAAGAAGATGTT | | |
| cg25771195 | GATAAGCGCCTAATATACATCCCTGCCTGTCATTATTCACATTGTGGCATGCAGT | 0.1700267 | Hypermethylated with Age |
| | CAAAG[CG]ACACTCTGAGGAAAATGTATCGCCTTAAATACATTGATTAGAAAAT | | |
| | AAGAAAGCCCGAAC | | |
| cg25828445 | GTGGTTTTGACGGTTGGGCCGTGTGAGTTGCTAGGACTCACCTGGGTCTCCAGTC | −0.02057556 | Hypomethylated with Age |
| | AGTGC[CG]GGCTGCCGCCCCTGCCGCCGCCGCCGCCGCCCCTGCCGCCGCCGCC | | |
| | GCCGCCGCCGCCCC | | |
| cg25954627 | TTTCCGCTGCACCCCTACCTACCCAATTTTTATCTCGCTACAATCAGGGGTTTTC | −0.0411702 | Hypomethylated with Age |
| | TCAAC[CG]GGGAAAAGGTGGTAGTGGCGGTGGGAGGGTCAGGATCCCTCGAAGT | | |
| | GGAGGCCGGGGCCG | | |
| cg25961903 | AATCTACATGTCTAATAACAGCAAATCTGCTAAGAAGCATTAGAAAGAGGAGCAG | −0.06767351 | Hypomethylated with Age |
| | CGCCC[CG]GAATCTCTGCAGGAAATGTTCTTTATATTAGACATATACAAGCAGG | | |
| | TCCTGTCAGATGCA | | |
| cg26002713 | ACAGCCTGGGCTCAACCTCTCCCAGACTTCCTGGACTCCAGTGGAGCCTTGGGCC | −4.05E−05 | Hypomethylated with Age |
| | GGGGG[CG]GGGCGTCCCTGGCCCCTCCCCGTCGTCCCGCCTGCCCGGAAAGGAG | | |
| | TGAGCGGCGCCTAG | | |
| cg26038582 | AAGCACCAAACTAGGCAGCTGGATAATGGGAGAGTCGGCTGGTTGTGAGCATGCT | 0.00562854 | Hypermethylated with Age |
| | CGCGC[CG]GGAACAGATCCACCCTCTGTTATTCCTCTGAATAAATATAAATCAC | | |
| | GATCAGAAACCCAA | | |
| cg26331945 | GTTGCGGGGCTCGGGGAAGTTACCCCCATCCGTGCTGGAGTAGCGGGGAAGCCCT | −0.36804163 | Hypomethylated with Age |
| | GGGTG[CG]TTACACTCGACCGTGATGGGGAGAGGGGACTTAGATGTTGTCACGC | | |
| | TGGGGGTCCCTTTA | | |
| cg26522278 | CTGCGATCTTCCCGTGCCTGAATATGAGGCTTGGAACAGACCCAGACCTTCCTGC | −0.14967169 | Hypomethylated with Age |
| | CTGCC[CG]TCCTGAGTGGCCCCGGGACCCCGCCCCATCTTTGGCCCCCAGCCCC | | |
| | TGCCTCTCTGCCGC | | |
| cg26614073 | CTTGGGCAACGTAGGAGACCTCCGTCTCCACAAGTAAAATTAATTAGCCGGCTGT | −0.23076484 | Hypomethylated with Age |
| | GGTGG[CG]CGCACCTGTGGTCCCAGCTACTCAGGAGGCTGAGGTAGGAGGATCA | | |
| | CCTGAGCCCGGGAG | | |
| cg26616148 | TTACCTTGGTGGCCCCCGCTTCCTCCCCACGGAGCCCGGCGCTCACCCCCGGGCC | 0.03350458 | Hypermethylated with Age |
| | GGGGT[CG]GGGTCGTGAGCCCACAGCTCAGCCACCATCTCCGTGTGAAGAAACT | | |
| | CAAACAACACAGTA | | |
| cg26657240 | CTGGCAGAGGGAAAGGGATTTTTTTTTTCTCCCTCCCTGCCGAGGAAAAAACAGT | −0.05168073 | Hypomethylated with Age |
| | TCTAA[CG]AGGAGTTACTTTGTAGTTTTAACTCATATTCAAATACTCCCAGCCG | | |
| | AGAAGCTGCCATCG | | |
| cg26685941 | AGCCCTGGTCACTGAGACAAAAGATACAGAGACACCGGGACACAGAGAGGTACAA | −0.28949261 | Hypomethylated with Age |
| | CGGGA[CG]TGGAGTTGGATCGAGCTGTCTCGTAAGGAATATCGGAAGGGGTGGG | | |
| | GACACTACTGGGAG | | |
| cg26767387 | GCCCCTCAACCCTCCTGGACCCAACTGTGCCCCCGCTTAGCTTCCAGTCCAGACC | −7.648−06 | Hypomethylated with Age |
| | TTCCC[CG]CAAATGAGTGTGTGCTGTGAGTGAGACCCCGCGTGTCTGCCCTTGC | | |
| | AGTCCGCCCTGAGG | | |
| cg26775176 | GCGGGGGCTTAGTCTAGGCCCGGCAGGGTTTTCTGGAAGACCAGAGGGCCACCAG | 0.0570563 | Hypermethylated with Age |
| | GTCAC[CG]AGGTGGGAAGTGAAGAGAGGTTCGACGCTGCCTCAGGCCTGGGCCT | | |
| | GGCCGGTGGGAGAC | | |
| cg26783079 | AGGGAGCGGCCACCCAGCCCGGGGCCTGCAAGCAGGCAGGCAGCCATTCGCCAGC | 0.00733259 | Hypermethylated with Age |
| | AGCCC[CG]GGCCCGGGCTGACTCACTGGGGGCCCCCTGCTGTGGCCTGGACCCT | | |
| | CACGCTATCCCGGG | | |
| cg26970841 | TGGCTGCCGGGGGGGGAAAGTGATTTCTCGGAAAGCAGAGCACTTCGAAGAAGGC | 0.14259673 | Hypermethylated with Age |
| | GGGC[CG]CGCGAGCCAAGCTGACGCTATTGGTCGGTGTGGCCGTCGCTCTGCGC | | |
| | ACCGCCCGTCCCC | | |
| cg26974111 | CAACTGCCCCGACCCCCAGAAGTGCAAAACGAACAGGCTGGCAAGTGACCAAAAG | 0.34377819 | Hypermethylated with Age |
| | AGACC[CG]GGGAGCATCTGGGCTTCCAAGGTCCTCGGTACGGCCCAAGGCAGCG | | |
| | AAGGACGCGCGGCT | | |
| cg26985354 | GGTCTCCACTGCCCTCCAGGCTCCATCCACCCGCCTGGTTTCCCGGGTCGCTGTG | −0.01068567 | Hypomethylated with Age |
| | GCCCG[CG]CTGGCGCTGCTGTTGATCTGCTGTGTGTGCTGTTCCCGAAGAGGCT | | |
| | CCGGGAGCCTGAGC | | |
| cg27045356 | GCACACAGAAAAGACCAATCAAGGACGGGTCATTCCCGCCCCCCGCGCGCCTTTT | 0.42866405 | Hypermethylated with Age |
| | GCGAC[CG]CCCACTCGACAGGTTGACAACCTAGACAGCTCCCCCGGACTTGCCT | | |
| | TACTTTTCCATCTC | | |
| cg27072218 | CTGAACCGGAGATCCAGAAGGACCCTCCAGGGATGACCTCCCAACTCTTTGCCTA | 0.2136334 | Hypermethylated with Age |
| | GAGAT[CG]GCAAGCACGTTGCCGGAGGCTCTGTCCAGAACATCCGCTCTGCGGC | | |
| | TCACTTTTCATGGT | | |
| cg27080085 | AATAAGTCATTATAAATAGTTGCTCCGTGGGCTTTCCGCATTTCACCCTGCTCCA | 0.00592303 | Hypermethylated with Age |
| | GGCAG[CG]TGGTCTGTATGGCTCCGCCCTGGGCAGGAGGAGAAAGAAGAGGGCC | | |
| | AGGCACCACGTGCC | | |
| cg27140880 | GTGATATATGTGAAATAATCCGGACATGCTTCTGCTATGGTTGTTTGTCGCCATA | 0.13534261 | Hypermethylated with Age |
| | GTTTG[CG]AATCTGGGTAAACCTGGATGAGAGAGACGCCTTACAATTGCAAACA | | |
| | TTTCTCAGGAGGCC | | |
| cg27200869 | GGATGTGCATAACTGAAGCTGCCTGAGGACTGGAGGACTGACCGGGGCCAGCACA | 0.06232967 | Hypermethylated with Age |
| | GCGGG[CG]GGGGCCTGAGGACTCGAGGACTGACCGGGGCCAGCACAGCGGGGGG | | |
| | GGCTCTGCCCATTA | | |
| cg27238852 | CTGCCCTTCGCTTGGTCTAGTTCGTGCTCGCCAAACCTGCCACCGTTTTGTGTCT | 0.22063082 | Hypermethylated with Age |
| | GCTCA[CG]GAACGTGATCTCTCTATACGCGTTAAGACGTTTGATTTGGTTCTTG | | |
| | TTCTGCTTATGGAA | | |
| cg27280366 | CGGAGGCCCGGGGTCCAGGGCGCCCCCTGCTGGGAAGCTGGGAACCAAGCCTTAG | −0.01845714 | Hypomethylated with Age |
| | AGGTC[CG]GCAGGTACAGATGGAACCAGGCCCAGGGCGACCCCTCCTAGGGAAA | | |
| | CCAAGGGTTTCTAG | | |
| cg27297851 | TAAATGCCGCGGGGGTGGCGCGCGGGGAGTGGGTTTGGGCACCCTCCTCGCCCTC | −0.57769479 | Hypomethylated with Age |
| | CCGCG[CG]GAAAAACTGGCACATAGATGCCGCCTGACTCCTCCAAAGCCATTAG | | |
| | AAGATTTGGGGTGG | | |
| cg27301488 | ACATTCCAGCCTGGGTGACACAGCAAGACCTGGTGTCAAAAACTAAAAACCATCT | 0.0158553 | Hypermethylated with Age |
| | CGAAT[CG]GCGCCCCCGCCACAAGCATTTTTTCTCGTCGGCAGCTCAGTTTTCA | | |
| | GCTGTGCTTAGTCT | | |
| cg27375378 | GCAGCGCTCGCCGCGCCTCTAGTGGGAGCCTCTGGCCTGGTGGTTTCCGGGGAGA | 0.20185114 | Hypermethylated with Age |
| | GAGCC[CG]AAGAGCAAGGGCCTCGGCAGCTTCCTCAGTGGGGCAGGGCCGGCGA | | |
| | TGCCAGCCAGGGAC | | |
| cg27442164 | TCCTCTTCTTTGTGAGAAAAGGATGAATCTTTCCTGATTTACTGTTGCCTCTTAA | −0.09279377 | Hypomethylated with Age |
| | ACACC[CG]TGGCAGGAATCTTTCTCACACCAGGGGCTTCTGTGTCATGCTGATA | | |
| | TGCCTGGAACTAGC | | |
| cg27636676 | ACCATGCCAAACCTGGCCGGGACCCGCTCCTGATCCCTTCACATCCACGGATTCC | −0.00320161 | Hypomethylated with Age |
| | CCCAG[CG]CCCCCCACCCCAGGGAACTCCTGCTAATCAACCAAGCTGAAACCTG | | |
| | GAAAAAATCAAGCC | | |
| cg27654505 | CAGCCCCTCGCGCGCCGAGGCCGCCGGAGCCGGGGTGGCCGCGAGAAAGCAGGGA | −0.32400067 | Hypomethylated with Age |
| | AGCCG[CG]CGCTGCAAGCTCAAGCCTCGCCGACGCTAGCCCCGAACACAAAGCG | | |
| | AGCGCCCGCGTCCG | | |
| cg27665659 | CGTTACCATGACGACCGGGCTCCTAGAAGCTGCAGTCAAGGACCTGGTTGCCATG | 0.45931104 | Hypermethylated with Age |
| | GTTTC[CG]CTTCTCCGCCTCCAGCCCCGCCCGCGCTCCCCGCGGCGTCGGCGCC | | |
| | TGCGCAGTGCGTGG | | |

What is claimed is:

1. A method of generating a differentiation-independent set of methylation sites comprising:
(a) determining the methylation status for different genomic DNA samples obtained from subjects having a range of ages for a large collection of DNA methylation sites to provide a dataset of methylation sites comprising methylations sites that exhibit age-dependent changes in methylation;
(b) determining the methylation status of genomic DNA samples from cells of the same lineage at different stages of differentiation from the large collection of DNA methylation sites to identify a subset the dataset provided in (a) that exhibit changed methylation with differentiation;
(c) removing the subset of DNA methylation sites identified in (b) from the dataset of (a) to provide a training set of differentiation-independent; and
(d) using a machine learning algorithm to identify CpG sites in the training set that have age predictive power.

2. The method of claim 1, wherein the cell of the same lineage in (b) are immune cells.

3. The method of claim 2, wherein the immune cells are cytotoxic T cells or helper T cells.

4. The method of any one of the preceding claims, wherein the subject are healthy subjects.

5. The method of any one of the preceding claims wherein step comprises performing dimensionality reduction of the DNA methylation profile of the lineage of cells along the differentiation pathway.

6. The method of any one of the preceding claims, wherein the machine learning classifier is elastic net regression.

7. The method of any one of the preceding claims, wherein the large collection of DNA methylation sites comprises at least 50,000; at least 100, 0000; or at least 200,000, or greater, sites.

8. A method of determining a biological age of a human subject, the method comprising analyzing the methylation status of at least 100 methylation sites set forth in Table 1;
comparing the methylation profile of the analyzed sites to a reference panel for the at least 100 methylation sites that correlates the methylation status with biological age; and
assigning the biological age to the human subject, wherein the biological age is the age associated with the methylation status of the reference panel.

9. The method of claim 8, wherein at least 150, at least 175, at least 200, or at least 250 methylation sites set forth in Table 1 are analyzed.

10. The method of claim 9, wherein at least 300 methylation sites set forth in Table 1 are analyzed.


SEQUENCE LISTING (536 sequences)

SEQ ID NO: 1 - DNA, 122 bp, Homo sapiens
  source 1..122: mol_type = genomic DNA
ccccttgggacaatgcgtaggggacctccgcgtccccgacacccgactgggacacggccgcgggctccttcgtccctcaccgccagccagggaggctctgcatgcccacgtccacttcacag

SEQ ID NO: 2 - DNA, 122 bp, Homo sapiens
  source 1..122: mol_type = genomic DNA
agcagggagcaacagacaagcccaaattgctgtgtttaaaggagcagggctgtctgtttgcgtggggctgtctccctgtaatgagaaccaccgctgagagctgtttagacaaacgggctcag

SEQ ID NO: 3 - DNA, 122 bp, Homo sapiens
  source 1..122: mol_type = genomic DNA
agtccggggtcgccgcagcccgggaggagtgtctggtctccggcctgcctgtgctgtccccgcgccctgtccactggactcccgagacccttggaacccaggtaacccggggcgggactccc

SEQ ID NO: 4 - DNA, 122 bp, Homo sapiens
  source 1..122: mol_type = genomic DNA
gtgtgagggagggggaaccggagctcaggagagggatctggccacaaagatgggtgggggcggtgcacagaggattctaaagacacagagtggcacagagggcagagagcctgtggagatga

SEQ ID NO: 5 - DNA, 122 bp, Homo sapiens
  source 1..122: mol_type = genomic DNA
caggactgcagaactggcccagacctctgtattggaaaggtctttatggaccagggagtccggtgtcttttttacgggggacccctgggctgcgagttgcacagtccaattcgctgttgtta

SEQ ID NO: 6 - DNA, 122 bp, Homo sapiens
  source 1..122: mol_type = genomic DNA
caggctccagcaaaatggcgccggcgccgccagaaatctcctggcctcctcagagcacgacgtaaagggggcgggcgtctctgtgacgtcacgaggctccacctcccgcgaggctttgtgtc

SEQ ID NO: 7 - DNA, 122 bp, Homo sapiens
  source 1..122: mol_type = genomic DNA
tcacaccgcagaacctttctgccttttactacttttcaaagtggacctaggctcctggctcgagcctgaggggatacaagggatcacgaggagcgccatcaccgtgcaaggtcgacagcttc

SEQ ID NO: 8 - DNA, 122 bp, Homo sapiens
  source 1..122: mol_type = genomic DNA
ctaatgtccaaaagcattcctaataccaagcataaagaatgtttcttaattcaccagactcgccaataagatagtgacatacactaagtcttcctggttatttaaaaaaggaggggaggggg

SEQ ID NO: 9 - DNA, 122 bp, Homo sapiens
  source 1..122: mol_type = genomic DNA
cgcggggcggggaggaggtcccaggagccggttcgaaagctccctccgtgatgaagtaggcgagaagggaggaggtgaaggagggcgagctgagcacacgcgcttcatgccacaggagggtg

SEQ ID NO: 10 - DNA, 122 bp, Homo sapiens
  source 1..122: mol_type = genomic DNA
agagcgaaacttcatctcaaaaaaaaaaaagaaggaaataaatgaaataagccacaagagcgatagaggagtaggacagatggagtgtaaagaaggagaagacattatatattttctctttt

SEQ ID NO: 11 - DNA, 122 bp, Homo sapiens
  source 1..122: mol_type = genomic DNA
cgtggagagtggagacaaagatgctcaaaagccaggaaatcgcaggctcggaagcccccacgcatgcctctgaacgcagcgccatctcggggctgcggcgggaccaagcgggacgcttgcag

SEQ ID NO: 12 - DNA, 122 bp, Homo sapiens
  source 1..122: mol_type = genomic DNA
aagtttgagtggggatttgctttctggaattgactgtctcctgtcttcagatgactgccccgtgtcagcccggcggcaatcgttagtctccgggccaacccaggacgatgctttttggcgtt

SEQ ID NO: 13 - DNA, 122 bp, Homo sapiens
  source 1..122: mol_type = genomic DNA
caacgccggctctgggcgggctctgggcgggctctggctgggctctggctgggctctgggcgggggcgggaggtttgtggcattggcgccagggttcttagccctcgcgacacagggccctt

SEQ ID NO: 14 - DNA, 122 bp, Homo sapiens
  source 1..122: mol_type = genomic DNA
ttcctggcggtcctcggcggagcgggagcagtgggacgtttccgggggtcgggtgggtagcggcgagcgctgtgcggtcagggcggggctcctgtgccctgtcggtggcgcagggagctgga

SEQ ID NO: 15 - DNA, 122 bp, Homo sapiens
  source 1..122: mol_type = genomic DNA
tttgtgctttttctcatacagagtatctccctgtcatagaacctacacaaagcagtgtgccggcccccaagaagtctgaacacctgtggcagacagcagcgtcaagcagctgtggttaaagc

SEQ ID NO: 16 - DNA, 122 bp, Homo sapiens
  source 1..122: mol_type = genomic DNA
tccctcaggactgggcacctcgctgcccccgctgctgcccactctgcgactgtgcctgtacgtgccagctccccgactgccagagcctcaactgtctctgcttcgagatcaagctccgatga

SEQ ID NO: 17 - DNA, 122 bp, Homo sapiens
  source 1..122: mol_type = genomic DNA
agggcggcactgagatttttgtcctgggcggcagacgaccttgtgttgcacttcctcccccgccttctgcctctcccggggcggcggcggatgggcgcggaggcggatgggcgcggctccct

SEQ ID NO: 18 - DNA, 122 bp, Homo sapiens
  source 1..122: mol_type = genomic DNA
tcctctgccatcatttgatcctctacccgctaaaaagcgggttttccttctgggacttggcgcaagcgctcctaggccaggcgcgcgcttaggtctgagaccggccgaggagcaggggcgcc

SEQ ID NO: 19 - DNA, 122 bp, Homo sapiens
  source 1..122: mol_type = genomic DNA
tgaccctctgactaaggatcaccgcagatacttaatcgccaagctgcccttgccttgggacggcacccaatcccaaagtaatctcttgctgctcctaaactggccacagccaagaacctcct

SEQ ID NO: 20 - DNA, 122 bp, Homo sapiens
  source 1..122: mol_type = genomic DNA
ccagcgcgccggggctggaacacaatgtcccgagcgggcgggcgggcgggcgagcgcgagcgagaacagcctgactcagcagctgggtaagtgggtgtgctcgctcaccagatcaaccgctc

SEQ ID NO: 21 - DNA, 122 bp, Homo sapiens
  source 1..122: mol_type = genomic DNA
agagtgtggcgagaacccccagggaccccctcccagggctggccttccaccctccgcccccgccaaccccacccgcattccagcgggatcagggggattaaagggcacagcatgtgtgggca

SEQ ID NO: 22 - DNA, 122 bp, Homo sapiens
  source 1..122: mol_type = genomic DNA
ttcactgccggggacctcagtttgcccatctgttaaaggagcatgttgaaccagaggacccgccaagccccttccgagtgcctacatgtaatcctccctcctctctcctggaccacagcgcc

SEQ ID NO: 23 - DNA, 122 bp, Homo sapiens
  source 1..122: mol_type = genomic DNA
caggagggaccttgaggaggaagggactcctcggtcataccaactcaggaagtggaactgcgtcacggtgcgcgtctcgttggtctgcaggttcttcagatagaagctcctcaccaggaagt

SEQ ID NO: 24 - DNA, 122 bp, Homo sapiens
  source 1..122: mol_type = genomic DNA
cagccatctctggagggttgaccccaataaacttcacatgaaaacaaatcatccaaaagacgcaggtgaaagtatataccacttatactgaagtctttttaaagtaaatcaccatatagtca

SEQ ID NO: 25 - DNA, 122 bp, Homo sapiens
  source 1..122: mol_type = genomic DNA
tttcttgttcttgccgcccatgttgcagctgtggcagaagatccttcgcggcccaggccccgacggtaccactgcacagccgagagctcttcacattccccggctccggggctgccaccctg

SEQ ID NO: 26 - DNA, 122 bp, Homo sapiens
  source 1..122: mol_type = genomic DNA
acggccagcagccgcgggaggggcacccagccttggtctgccagccacccgcagaccagccgggcgcccgcatcccccatcgcagccacggccaccaccagcgctgctccgtatccccccag

SEQ ID NO: 27 - DNA, 122 bp, Homo sapiens
  source 1..122: mol_type = genomic DNA
ccaagggcctgacatcacaaggggaggggaaggcagctgaggttgtggggggaggtgccccgccccttggcaggcccctacagccaatggaacggccctggaagagacccgggtcgcctccg

SEQ ID NO: 28 - DNA, 122 bp, Homo sapiens
  source 1..122: mol_type = genomic DNA
cacgtagcgccggctgcgcgacagggcgcgcgcgacgctgccgacggcgcatgcgcggtccggcatgccgggcggaagccccatttgatttctaatgctatttatttatgtatcccttgtct

SEQ ID NO: 29 - DNA, 122 bp, Homo sapiens
  source 1..122: mol_type = genomic DNA
ctgggcaggggatgagcttgtgtcgcgggcggcagggggaagggagtcggagagctcctgcggtccagccggatgactgatgaggttgaaagcacttccgctgcggcccccgcaggaagttc

SEQ ID NO: 30 - DNA, 122 bp, Homo sapiens
  source 1..122: mol_type = genomic DNA
taaaggaggggggacgccagcaaagcgggggcacaggggaggggcgcacgcacacgcacacgcacacatgcccgcactcacagatggagattctgtattggcaacttatccggaatctcaac

SEQ ID NO: 31 - DNA, 122 bp, Homo sapiens
  source 1..122: mol_type = genomic DNA
gggtgtctctccctgttttacggttgagggccttggtcaagccctgcagccttgaggttccgttcctcatccaggaagcccgcgaggcacgctgcaggcaggtccaccggcccgcgcatccc

SEQ ID NO: 32 - DNA, 122 bp, Homo sapiens
  source 1..122: mol_type = genomic DNA
tgcctggaatggacccttggagggtatggcactacaccttattaactgttgctaaactgtcgctgaaacacacatgtccgtttacattcctgcctcagtttctgtttccccaggcccccgcc

SEQ ID NO: 33 - DNA, 122 bp, Homo sapiens
  source 1..122: mol_type = genomic DNA
tcatcaccttgtggccagacaggatattgctgttagagactccaagagcctgtttgggttcggagctattctggtcaattttatcaccccatgcactgcctccacttactcatgggccaggg

SEQ ID NO: 34 - DNA, 122 bp, Homo sapiens
  source 1..122: mol_type = genomic DNA
ctcaaaggtaattttcagctgtgcctcatgaggggctggtgtaggaactgagaaccaccacgatggtgtcctggcttctctgtctgggtctgctcatgtcacgtctattctctatggctttt

SEQ ID NO: 35 - DNA, 122 bp, Homo sapiens
  source 1..122: mol_type = genomic DNA
ctcgggtgatccagccacctcgggttcccgaagtgctaagattacaggcatgagccacggcgcctggccaggatttttaaggttgaagcatccacaacaattttgtgtgtgaaatgaaatga

SEQ ID NO: 36 - DNA, 122 bp, Homo sapiens
  source 1..122: mol_type = genomic DNA
cgtctgccctggccagggaaggtgcccagaggcgggaggccggccggatcacacggacctcgcaggcccttcccagacgctgggttctgaccccgtgggggcccctccctgcccagttctgc

SEQ ID NO: 37 - DNA, 122 bp, Homo sapiens
  source 1..122: mol_type = genomic DNA
ttccttttagggtgagcctttggttctcctgtcccaaaccgaggggggccacgtgggagccggcagcacacactggccaccactgaccgctcctgctgtcctggcacttccgccccctccca

SEQ ID NO: 38 - DNA, 122 bp, Homo sapiens
  source 1..122: mol_type = genomic DNA
aacgttcctttctggcacgaccagtatctgaacatctctcacaatacccacgccacagtacgagacactgtagcctaagagtaaagcctaacagtcagctcctagcattagctttggagtta

SEQ ID NO: 39 - DNA, 122 bp, Homo sapiens
  source 1..122: mol_type = genomic DNA
aaacttttatttacactgggtggcacacccctgaggcccacacccatcacccaggagtaccgcaatcttcctgatcccctggactcacctgggcggggtgcagactgcgaaggcgatagtac

SEQ ID NO: 40 - DNA, 122 bp, Homo sapiens
  source 1..122: mol_type = genomic DNA
gcgagggaccgagtctgggagagccttccgagtccggcctcctgccttcccgccatgggacggctcgccccaatcccgcgaggctcctgcgcctccgcaacccaggcccgcacccctttccc

SEQ ID NO: 41 - DNA, 122 bp, Homo sapiens
  source 1..122: mol_type = genomic DNA
ccggtgcgaagccaccgagccgggtccgcagccctactgtgcacctgcatcctcagtccccgagatcagtccccagggcgtcagctggggagtcaaggagtcagcctgctccgggctgacct

SEQ ID NO: 42 - DNA, 122 bp, Homo sapiens
  source 1..122: mol_type = genomic DNA
aggcattcatggacattcttcttagaaggcctagtgctaagtcgcagttgtcaagggaaccgcgtgtctggagggagaacaggctctccggagtttcccgggaaaccaccccccgcagaggc

SEQ ID NO: 43 - DNA, 122 bp, Homo sapiens
  source 1..122: mol_type = genomic DNA
tacacttcacaaacgcactagtgcccactgtctttatctggaaggggttgagtaggttggcggggggcaacgctggtgtctacacagccaagagggaacattcacgcacacagaatctatga

SEQ ID NO: 44 - DNA, 122 bp, Homo sapiens
  source 1..122: mol_type = genomic DNA
gccatggcgacagtctctaggcagcgtgggctgagctctccgtaggaataaagggtgggccgccgctggaccctgcgcgcgcccgccaccaactttccctccagatccgagaggggcggcgc

SEQ ID NO: 45 - DNA, 122 bp, Homo sapiens
  source 1..122: mol_type = genomic DNA
tggctcggcagcccccagccccgccctgcggccaggcacacatgcgggcacaggcaggggcgccagaaactcaactagaggacacagcagcttcaggaacactggtgaattccgccggactt

SEQ ID NO: 46 - DNA, 122 bp, Homo sapiens
  source 1..122: mol_type = genomic DNA
actccatggacgcagcgacgagagtgatcagcaggagtccctgcacaaactgttgacatccggaggcctaaacgaggatttcagcttccattatgcccaactccagtccaacatcattgagg

SEQ ID NO: 47 - DNA, 122 bp, Homo sapiens
  source 1..122: mol_type = genomic DNA
gtcaagcccctgtcaagccacctggtgctccgtgtcctgtggatggtagctgccctgaagcgggactttgcagactgaagtgctgtctcttcagagggagtgcaggtgtccggctcctggtg

SEQ ID NO: 48 - DNA, 122 bp, Homo sapiens
  source 1..122: mol_type = genomic DNA
tccctaagccccggcagccgattcggagactcgggaggccacaggctcagcgcgacaccacgaccacaactaggaggcaccatcgtcgatctacctggggaggcacctacaaagccagcaga

SEQ ID NO: 49 - DNA, 122 bp, Homo sapiens
  source 1..122: mol_type = genomic DNA
ctcctcgatgatggcgtccagctcctccttggtgggtgtctggcccagcatcctcatcaccgtgcccaactccttgacgctgatgtccccaccaccatcagcatcaaacatgtcaaaggcag

SEQ ID NO: 50 - DNA, 122 bp, Homo sapiens
  source 1..122: mol_type = genomic DNA
agagctcaaactccatcccttggctcaggtttgcccctttcatccttctccagtgctctgcgatggagaaagaaggcaccaccataaatgaggttgaacacctggggctctgactgggttta

SEQ ID NO: 51 - DNA, 122 bp, Homo sapiens
  source 1..122: mol_type = genomic DNA
ggtggcactgatagcctgggagagggagggagaggaggacgggggcacctactcaggctccggctgcctcaccacaagaggaaaacagggtgaagttgcaggaaaggagccccaggccgggg

SEQ ID NO: 52 - DNA, 122 bp, Homo sapiens
  source 1..122: mol_type = genomic DNA
gggccagcagctgctgtgctggatttctggggtcttagctgtcccagcggggcagggttccggacctgcacccggccatggccatgcctgagcctccccctgctccccccgctccccccacc

SEQ ID NO: 53 - DNA, 122 bp, Homo sapiens
  source 1..122: mol_type = genomic DNA
ccagagagcacatcttgccggttcgcaggacgtctgcagtcggcaaactcctggccggaacggcacagaccgcactcccgcaactcggttcccgggctagattcgtatgcggacgggtaccg

SEQ ID NO: 54 - DNA, 122 bp, Homo sapiens
  source 1..122: mol_type = genomic DNA
ggcacccgtctccacctccccagcagctgttccctgtcatgtcggtgcatttacaatgagcgccagtcgcctgtctccaggtggtcagaggttgaaatcccttttgaaaagttctttaaaaa

SEQ ID NO: 55 - DNA, 122 bp, Homo sapiens
  source 1..122: mol_type = genomic DNA
ccgtccgttttccgcccacttgggcccagccgtccaatcgacactcatcatgctctgcctcgccgctctctccggccaatccgcatgtgccactgcctctgcccgcaatcggcgctcaccaa

SEQ ID NO: 56 - DNA, 122 bp, Homo sapiens
  source 1..122: mol_type = genomic DNA
caggatcggccgggaggcgagagggattttgttgaggagcaaggtcttccacaggaactgcgacttggaaagtattcaccaagggctgtgccatgcgaaaccctctttaaaggaaccgcatc

SEQ ID NO: 57 - DNA, 122 bp, Homo sapiens
  source 1..122: mol_type = genomic DNA
atttactcctctttcctgaagctttgcctctagacttacttaactctttcctccagcctacgttcatcgaaactatcacttattgtcatatagacctttatttctgaagggagatctgggag

SEQ ID NO: 58 - DNA, 122 bp, Homo sapiens
  source 1..122: mol_type = genomic DNA
cagggtcaccacctggtggtacttcttgaccagggccacgatcagggccttctgcttgggcgtcacgcggcagcagatgaccgcctggcacttggacgccaggtccacgaaggcgcgctcct

SEQ ID NO: 59 - DNA, 122 bp, Homo sapiens
  source 1..122: mol_type = genomic DNA
ccacacctctcccctgcccagtatctcccccattctaccccagcccatggccttgcgccacgctgcttcatccatcctgatgcccagtatgtaaaaggcgcctaacccgccatcatgtccct

SEQ ID NO: 60 - DNA, 122 bp, Homo sapiens
  source 1..122: mol_type = genomic DNA
ccacaaagtctgggggcggagcagattgggtaccaaggaggactgcctggaggtggtggccgcggtgactgctccaaacccgttcggccctgtgcagaagtctgaagaacagaggtgccctc

SEQ ID NO: 61 - DNA, 122 bp, Homo sapiens
  source 1..122: mol_type = genomic DNA
ctcctaggatacaggctgggcaaggcagataagtggctcttggcctggtgacctttccagcgtccagttctttggagtaaccacttcgcagagcctcatccagccggaggagccccaggcta

SEQ ID NO: 62 - DNA, 122 bp, Homo sapiens
  source 1..122: mol_type = genomic DNA
ctatattagggctttgttgctgacaacagtgaaaacttgtttgtgtcaggaagtgaggtacggagatatgacctggaaggtacagacaaaaccaaagtggcagtttttgcattacttttctg

SEQ ID NO: 63 - DNA, 122 bp, Homo sapiens
  source 1..122: mol_type = genomic DNA
ccagcctggcaggggattttaaaatcggccaatcacagcgggcgccaggcctccgctttccgcttactggtcctgccgtaggaggcgggtacgtgagcgcaccaatctgtggccggcgagcg

SEQ ID NO: 64 - DNA, 122 bp, Homo sapiens
  source 1..122: mol_type = genomic DNA
agagagacacagcaaagtggggtgccaggcagaggccaggggctttcaaagaccggggctcgagttccgtcacagccacccctgtgacatcggccgtctttctgaccctccgtgtccccggg

SEQ ID NO: 65 - DNA, 122 bp, Homo sapiens
  source 1..122: mol_type = genomic DNA
tagcactcgtgacgtcaggtccaaatgagaaattgactggcactccgggccaatgggaggcggcgagagcggccgcgattagcataatagtatagaaacaaggaaacttttcggagctgtca

SEQ ID NO: 66 - DNA, 122 bp, Homo sapiens
  source 1..122: mol_type = genomic DNA
ttctctgatccgtaagagcctcctgcccgttcatccatcaacagcctctggggacaccttcggcagcaggaatcactggtgttgggtactgagtttgaatttctgtgcccacctttgcgctg

SEQ ID NO: 67 - DNA, 122 bp, Homo sapiens
  source 1..122: mol_type = genomic DNA
ttggtggccgcgcccgtcgctctttttacataagacgcacatggaactccatgttcacctcgtcggttcctcaatggagacgcggcgcgttcgtgctacccgtcgtcctccctagtggtctc

SEQ ID NO: 68 - DNA, 122 bp, Homo sapiens
  source 1..122: mol_type = genomic DNA
acacgcccgttccgcgtccgtctctggctccgcacttgctgccctctcgccgctcacattcgcaaagggggacagacactcatcggataatgacacagctggacgcagagccccggagagtg

SEQ ID NO: 69 - DNA, 122 bp, Homo sapiens
  source 1..122: mol_type = genomic DNA
gattggtgaacgtcgcctggcggtgtgtatgctttggaattcgtggtttcctctgggcctcgatacgccatccatttttgtgattgttccatgggcacttgaaaggccttcgatgaatattg

SEQ ID NO: 70 - DNA, 122 bp, Homo sapiens
  source 1..122: mol_type = genomic DNA
aacaaaccctccggcctggatcccaaaacaacagtcgcggttctgcaacagaaaaggctgcgctggccctgggacctgtctcggaaatactcctcatccatctagtttctcccaggacaact

SEQ ID NO: 71 - DNA, 122 bp, Homo sapiens
  source 1..122: mol_type = genomic DNA
atcagacttaggtcacagaattcaatggtttctgactattttatttaaactggaaatcggcgggatggcaaggaatactacttgcttctatagtgtgtgatccacattagtgatttgtggaa

SEQ ID NO: 72 - DNA, 122 bp, Homo sapiens
  source 1..122: mol_type = genomic DNA
ccacacacccttaaggtttttcacagcactctgacggtattatgtgtgttttgcaaatgacgaatcaacagtatgctgaataatcagcaatgaaacacaggagataaattaaatgtgttttt

SEQ ID NO: 73 - DNA, 122 bp, Homo sapiens
  source 1..122: mol_type = genomic DNA
gaaacagaatccccgcgtgccccttcctcactaccctccaaatcccgctgcagccattgccgcagacacgatgccgaaacgaaagaagcagaatcatcaccagccaccgacacagcagcagc

SEQ ID NO: 74 - DNA, 122 bp, Homo sapiens
  source 1..122: mol_type = genomic DNA
agtggacagtgccgtacagtaatgtctacggggagttccaggagagctcggctactcctgcgcaggataacctctcccccaccacccgagtcccgtgctcgcgggcaggacttttccgaact

SEQ ID NO: 75 - DNA, 122 bp, Homo sapiens
  source 1..122: mol_type = genomic DNA
gaagggggttcttaccgctgaggagaggccggttgtgccggtaggaggcgggcagctggccgacaccctccatgcggtggctcatgacgcgtgcgaggtggcccgtgtggtgcaggctgccc

SEQ ID NO: 76 - DNA, 122 bp, Homo sapiens
  source 1..122: mol_type = genomic DNA
gttgaagctgttccggaaggatctggactcgggcggcgcagcagaggggtcggggtcgggcggcggcggcagagcctccggcctgaggccccggaggaacgacggtctcggggagcggcccc

SEQ ID NO: 77 - DNA, 122 bp, Homo sapiens
  source 1..122: mol_type = genomic DNA
ctccgcgctcagcgggaggaggcgctcggtcccgcttcttacaaccagcggcgctcacggcgggcccggggatcagcatcccgggagcttctcaggaatgcagattcccaggccctcactgc

SEQ ID NO: 78 - DNA, 122 bp, Homo sapiens
  source 1..122: mol_type = genomic DNA
tccttcctcttgtcggggcctgggcttaggacacgcccgttccgcgtccgtctctggctccgcacttgctgccctctcgccgctcacattcgcaaagggggacagacactcatcggataatg

SEQ ID NO: 79 - DNA, 122 bp, Homo sapiens
  source 1..122: mol_type = genomic DNA
agtatgggagtttttaagaaagcttctaaaaggctgaaaagatacgcaaatgaagaaactcggggctcaacacaccactttaaaggactacaggatcctgtttgcagagtttagaatgaaga

SEQ ID NO: 80 - DNA, 122 bp, Homo sapiens
  source 1..122: mol_type = genomic DNA
cgcagccgctggcctgggtgctaagcccctcattgcccgcggcggcagggctggcagggccggctgcgctgccggctgttccgagtgtggagcccaccaagcccatgcctacctggaactcc

SEQ ID NO: 81 - DNA, 122 bp, Homo sapiens
  source 1..122: mol_type = genomic DNA
cccactgtggctgcaccccgaggaggagtccgtggcaggaggcccacacccaggacccggcgccaacagtgaggagacgaacctggaagacatcccgctgtccagacaccgcgagggaggcg

SEQ ID NO: 82 - DNA, 122 bp, Homo sapiens
  source 1..122: mol_type = genomic DNA
gtcggcacacagaggtcgctgcagaagcctagctgtggctgttcttagagcagcaagcagcgttctcgccctcatcctgattactaaggcatgaatagtgctgcttctgatggccgtttccg

SEQ ID NO: 83 - DNA, 122 bp, Homo sapiens
  source 1..122: mol_type = genomic DNA
cggtaggaagacttgaaaggggtggtcgaggcacgtttgtggtttgaaaggaaaaaaacacggagcagcagatgtcccccccaagggtgaaagtgcctcccaatcgcacatgcgagggtctc

SEQ ID NO: 84 - DNA, 122 bp, Homo sapiens
  source 1..122: mol_type = genomic DNA
gtggtcgggacaatttgcaactagagggtggtcctcatgggtaccctgtggggtgtcgcgcgaggtgaagggccaggccatttccgtcgggtcagcgatttccgccttcgccccgctctcgc

SEQ ID NO: 85 - DNA, 122 bp, Homo sapiens
  source 1..122: mol_type = genomic DNA
tgggtgacaaaaatgaaactgttttagaaaaaaataaattttactttgttagaataccaacgcaaggttccttagaaggttgttgcagcatctgccctctatgggcagctgtacagtgacta

SEQ ID NO: 86 - DNA, 122 bp, Homo sapiens
  source 1..122: mol_type = genomic DNA
ctgggcagagggtgcggcaaggctcacacagcaggcaggctgcagctccaggagaaagcccgcaggcctcaaacagaagacgcagtgatgcatccacaccaggaaaaccaagcagccttctg

SEQ ID NO: 87 - DNA, 122 bp, Homo sapiens
  source 1..122: mol_type = genomic DNA
gtcagcatgaatagtgctaaggagtcaagccaagacttgcttcagaagagagctgatgtgcggaccttctgcatttccacgcggggatccagtgcttgggtctaaaccccagcgccctgcca

SEQ ID NO: 88 - DNA, 122 bp, Homo sapiens
  source 1..122: mol_type = genomic DNA
agagggagcccggcccctgggtgtgctcggcagctgctgagatgatggcaagggggagcgcggcacctgtcgtcgtcacctttgtgacgtcccaagcactggggggccctgcggggggtggg

SEQ ID NO: 89 - DNA, 122 bp, Homo sapiens
  source 1..122: mol_type = genomic DNA
tgaagaattgagagagaatgaaagtgccacaaaaacaaaagaaaaaaaattgaggcgagtcgtggacatgatagacatgattttgcaaacaaggcacatctaggagaaaaggcgggagaaaa

SEQ ID NO: 90 - DNA, 122 bp, Homo sapiens
  source 1..122: mol_type = genomic DNA
gttggggacgaactctttgctaaataaacatttaggtgaaacaaagcttccatttccaatcggcctaactctggtattagcgttggcaggggccatgcaggaggggcagtgagcagtggtgg

SEQ ID NO: 91 - DNA, 122 bp, Homo sapiens
  source 1..122: mol_type = genomic DNA
ggctgggaaacgctgattccctgtgggcaggaacgcttgtctaattgcgacggccctgcacggctcctgcccaggtatgaagtgcagctggcttgcaccagtgcgagggcggagacgcgctg

SEQ ID NO: 92 - DNA, 122 bp, Homo sapiens
  source 1..122: mol_type = genomic DNA
ccgtattttccaccgcgctgtattagtgagggctctttgtgtcacttctgtgcataactccgccccaagttaaaaggctcgctgctctcgacaggtcctcctcctcctcccgctaggtcctc

SEQ ID NO: 93 - DNA, 122 bp, Homo sapiens
  source 1..122: mol_type = genomic DNA
aaaccccgttttaccccaagggtggatagaagggaaatgctgagttttcatgggatctcacgccaggagaaaatcaggagcatggaaggggtgcaagttcatggcaagacgggacaggactc

SEQ ID NO: 94 - DNA, 122 bp, Homo sapiens
  source 1..122: mol_type = genomic DNA
gcggcggcccgcggcgcagcggggcccgggccgggaccgccgtcggggggcgcggcgacgcggagcccgctggtgaagccgccctactcgtacatcgcgctcatcaccatggccatcctgca

SEQ ID NO: 95 - DNA, 122 bp, Homo sapiens
  source 1..122: mol_type = genomic DNA
tgggcctcaagcacttcacaacatgattattttattcctaacggtcagtagcgaataagacgtaaaatgacatagttctgctgtgggtaaactcaaggttttaaaaaggaaaaacttcattg

SEQ ID NO: 96 - DNA, 122 bp, Homo sapiens
  source 1..122: mol_type = genomic DNA
gcccaaacgctggctcaggctaaggccaggtgggctaatctcaatgttggcctgtggtggcgaaagtcttgtgcaaatggcatatgttaggaatcactcccagaggcaggacccttgaaggc

SEQ ID NO: 97 - DNA, 122 bp, Homo sapiens
  source 1..122: mol_type = genomic DNA
cggtctcttttcggggtgggaggtctcccactgggaccgacacagacgcactggatgccgcggggtcccgggctccgagcggcggtgtccctgtccccttactcctacccacccccacccac

SEQ ID NO: 98 - DNA, 122 bp, Homo sapiens
  source 1..122: mol_type = genomic DNA
tgcaggtcagcaaacatttcggagcacccaatttgtgtctggtcctcgcttattgcaggacgacccgggttatcggacacccccccttcccaccccaatcccacccaactcctgtcacctta

SEQ ID NO: 99 - DNA, 122 bp, Homo sapiens
  source 1..122: mol_type = genomic DNA
acttggaatgaacatgttggaaataaacgctctcattttgcaggcagataaactgggaatcgtgcgtgtaaagcagcttgctcaaagtcttataactatgaattggaaagtcagattcgagc

SEQ ID NO: 100 - DNA, 122 bp, Homo sapiens
  source 1..122: mol_type = genomic DNA
gagaagaaaccgaggatgttcaagctggagaagggggagggggagggcggaggaaggactcgatcactgtcttcgtagctgaagggctgtcaggtggaggtaggatagacttgttctagctt

SEQ ID NO: 101 - DNA, 122 bp, Homo sapiens
  source 1..122: mol_type = genomic DNA
gaaggagccggaaccggagcgggcaggacctgaggcttccctcgccggggcaacggctgccgccgcaacccgggtcccaccagcgccgctccacctgcaacggtccctcaggctttaggaga

SEQ ID NO: 102 - DNA, 122 bp, Homo sapiens
  source 1..122: mol_type = genomic DNA
ccgcctccacccttgacttgaatcactgttggcgggggacggggcgtgacccattcatgccgggaatcggatccagatgttcccgcggcgtgtgcagctgcatccttgccttttttggcaaa

SEQ ID NO: 103 - DNA, 122 bp, Homo sapiens
  source 1..122: mol_type = genomic DNA
gcccctctgctccggctcggggcgggcactggcggagggactggccagtcccctcctccgcgccggccccaaccctgtcgctgccgccgcgctccgagtccccattcccgagctgccgctgt

SEQ ID NO: 104 - DNA, 122 bp, Homo sapiens
  source 1..122: mol_type = genomic DNA
tgctggtggcggctgcagctgcagcgcccgtgggctgacgtggcttcccggagctgcggccggccagcgcccaagggcccacaggctgcgctgcccttgccagctgcttctgacccgcgccc

SEQ ID NO: 105 - DNA, 122 bp, Homo sapiens
  source 1..122: mol_type = genomic DNA
tgcacgcagcatcggccctacacctgcttccactggcacttcgtgaaccagcggcgccgccggtccatccgccgtcgggacggcaccttcaattacagccctgacgtctactgcaccaagta

SEQ ID NO: 106 - DNA, 122 bp, Homo sapiens
  source 1..122: mol_type = genomic DNA
gagcggccccgcggaggagccaccggaggctgtagttgccggggagtcccgcattcagtccgctcagcctctgggccgggcctcggcggcccccagagccccacacccgcaggcccagggcc

SEQ ID NO: 107 - DNA, 122 bp, Homo sapiens
  source 1..122: mol_type = genomic DNA
ctctgcggggacagaggtctcaggaaagtagcctttatttatgtggcaccgatcggaacccgcggccggccaggcggacctggacggagcgtccctgctcggaacctggcgcggggcgccgc

SEQ ID NO: 108 - DNA, 122 bp, Homo sapiens
  source 1..122: mol_type = genomic DNA
ggagatcttttcagaccaataaccttccctgcctccaaaacaaaatggggaggtagagggcgctagcgatggaacagatgttcctgcacgtcttacggatggtccagggtgggttttgtgct

SEQ ID NO: 109 - DNA, 122 bp, Homo sapiens
  source 1..122: mol_type = genomic DNA
cggcgcgcgccgggctgtagctctgcgacgacagcgagcggttctgctgcgggtacgtggcgcacggccgcagcgcccccacggccggcgcgcacgcctcgtcccgcgcgcccgacgcctgc

SEQ ID NO: 110 - DNA, 122 bp, Homo sapiens
  source 1..122: mol_type = genomic DNA
gggacgccgagcggagctctcggagctctcggggctctaggggcctggggctagctgctccgcggcgcggggagctccgggggtccaaggaggagccgccgccgccgccgccgtgacgctgg

SEQ ID NO: 111 - DNA, 122 bp, Homo sapiens
  source 1..122: mol_type = genomic DNA
tctgcagaatctggaccggctgacatttgaacccctagcaaacctgcagctgctgcaggtcggggataacccctgggagtgtgactgtaacctgcgtgagttcaaacactggatggagtggt

SEQ ID NO: 112 - DNA, 122 bp, Homo sapiens
  source 1..122: mol_type = genomic DNA
cctgggtggcggcggcggtgagggctgcgagcaagggtgctgcgtttgcattcgggggggcgggtatgtttctgcttcaggtggagctctgtagcgtttcattatcaccccagaaatcctca

SEQ ID NO: 113 - DNA, 122 bp, Homo sapiens
  source 1..122: mol_type = genomic DNA
gcaccgagcgcccacggctccctacgggagctgggccccccgggcctccaggtttcggcccgccccctggcaggcagcacaggtggctgagcaccgctacagcggcctctcaccggccgctt

SEQ ID NO: 114 - DNA, 122 bp, Homo sapiens
  source 1..122: mol_type = genomic DNA
ctggcggtagaagcggcactgcataccaacacaagacgttattttaagcgcgtgtccccacgagagaacccatccgatctactggagcaagcatctcccacccgccgggaattttccaaagc

SEQ ID NO: 115 - DNA, 122 bp, Homo sapiens
  source 1..122: mol_type = genomic DNA
gtattgggactcgctggcgtagggatgctgcgctcaagggtgcgacgccaactgggctcgcgcaggcgcgcgccgtcgagcgggagcgggacacctgggctcctccttggcccctccccgca

SEQ ID NO: 116 - DNA, 122 bp, Homo sapiens
  source 1..122: mol_type = genomic DNA
tatactttaagttctagggtacgaggccagggagaaggagaagccaccctgaggaaggtgcggaatgtcgcgtggagcccggctctctgcctttgaagcaggattttcatgcactcgccagc

SEQ ID NO: 117 - DNA, 122 bp, Homo sapiens
  source 1..122: mol_type = genomic DNA
cctcgcgctactcaatgacgaggcagcggggcaggtgctgcgagaaatacttgaagagctcgggggtggccccggggcagttggtcagctccagctcctccagctcctgcagctgcaccagg

SEQ ID NO: 118 - DNA, 122 bp, Homo sapiens
  source 1..122: mol_type = genomic DNA
accggagcccgcgggggcggcagagacccgccccggcccgcaggacaccccctcggaacgcgcggccccccggctaagtcatgtttaacagcctcagaaattatcttgtctccgcgttcttt

SEQ ID NO: 119 - DNA, 122 bp, Homo sapiens
  source 1..122: mol_type = genomic DNA
aaggaggagatggccaagggcgaggcgtcggagaagatcatcatcaacgtgggcggcacgcgacatgagacctaccgcagcaccctgcgcaccctaccgggaacccgcctcgcctggctggc

SEQ ID NO: 120 - DNA, 122 bp, Homo sapiens
  source 1..122: mol_type = genomic DNA
tcagccatgagggcatcacggcagccctgaggcctgtgcgggtgcccggctatgccgactcggatcccaccttctcgctgagtgtggatgaggactatgacctccgcctgtctggcctctcg

SEQ ID NO: 121 - DNA, 122 bp, Homo sapiens
  source 1..122: mol_type = genomic DNA
gggctgcgtcccttttcagcagctgtgttcggagctgagcagcttggagccacacacgtgcgttatctcagggttccgggacacgcgtgctgggggcgctgcccgggccctcactaggcggg

SEQ ID NO: 122 - DNA, 122 bp, Homo sapiens
  source 1..122: mol_type = genomic DNA
cacgtcccaggtgtgtgtccgaccgtgtgtgtgagtgtgagagaagaaataaaaagcccccgtctcccaaaagccctggcaaaccagcccagctggaaaatcctaaatgcaggctcatcaga

SEQ ID NO: 123 - DNA, 122 bp, Homo sapiens
  source 1..122: mol_type = genomic DNA
gaaaaaaagtaattttttaaaaaacataataaatcctcaattgtccccactgaaagggctcgagtcttttttaaattccaaagtgaatttatgttctgtaatttgcattacagccaagcgat

SEQ ID NO: 124 - DNA, 122 bp, Homo sapiens
  source 1..122: mol_type = genomic DNA
attgaggtaccatatgcccagtttcttcctagaatctattctcactgcgttcaccgtttgcgtcccagcactcccttcgtctgatttgtagcactccctgtacctgccccggctgcgttgca

SEQ ID NO: 125 - DNA, 122 bp, Homo sapiens
  source 1..122: mol_type = genomic DNA
cagtatcactattggcattcctgagccactggctcagaatttcagtacattatctgcccgcgggacacacctcagaggaaaggggatgaagcgtggtccatgaccatggcaccccctggtct

SEQ ID NO: 126 - DNA, 122 bp, Homo sapiens
  source 1..122: mol_type = genomic DNA
tagagcagcagctttgtttctttgcttcactgctgtctccctagcacttagaacatagtacgtgctcaataaatgaatgagtgcctgactgaatgaatgaggatacatagatgagattcctc

SEQ ID NO: 127 - DNA, 122 bp, Homo sapiens
  source 1..122: mol_type = genomic DNA
cgacccgatctttctgcccttgattcaaaacaatctgaggtccctaggcccttccctttccgcctctgcgctccccatggggtccggtgtagttttcccgccccttccctgcagctcccgag

SEQ ID NO: 128 - DNA, 122 bp, Homo sapiens
  source 1..122: mol_type = genomic DNA
aattctcctatggttctggagctcagaagtctgaatggtctttcgaggctaacatcactgcgctgtcagagctgtgtttctttgggaggctcttggagagaacctgtctcctcctcaccttt

SEQ ID NO: 129 - DNA, 122 bp, Homo sapiens
  source 1..122: mol_type = genomic DNA
ccagtcccactctgcttaactgctctggcatgcttgaaggcctagcttagcgtagcaggccgttgcagccgttctcgctctgtggcattgctctttgccttcttggtccagctgcctccagc

SEQ ID NO: 130 - DNA, 122 bp, Homo sapiens
  source 1..122: mol_type = genomic DNA
cagcaaagctgcccttagacgagagctttcgcaccgctggcgcctcctgttgcgcgcgctcgatggagaagtggtcccgacgcgcgcgtcgactcttccagccttgagaggctagcggcgcg

SEQ ID NO: 131 - DNA, 122 bp, Homo sapiens
  source 1..122: mol_type = genomic DNA
gaaagccgccaaggtggcgcccatctgagcgacaggagggagcggccctggcaggacggacgtgggaactgcaggggcacaggccctacgtgagctgcgtgggtggaaaccgaggctgggac

SEQ ID NO: 132 - DNA, 122 bp, Homo sapiens
  source 1..122: mol_type = genomic DNA
cggggctggccaggggggaagggaggggagaagagggagccgggcgtctcagcgcgggagcgggtttcagggtccccgggcccctcctcgcgccccgccgctgactataggggcggggccgc

SEQ ID NO: 133 - DNA, 122 bp, Homo sapiens
  source 1..122: mol_type = genomic DNA
ccgctttccaaaaggctttagtggaaaacaggtccagggtgggcccagtggagtgggccccggaggcatggggcacggggcttaggagaatattcggatggcttgcgtggctgtgatgtggc

SEQ ID NO: 134 - DNA, 122 bp, Homo sapiens
  source 1..122: mol_type = genomic DNA
gctcaaacacactcaagccccaggacacacactggcacagacacgtacatgcatcctgagcgctgggactctcacgcatgccacctgccattgcaacgccctcccagctgagccaggggcct

SEQ ID NO: 135 - DNA, 122 bp, Homo sapiens
  source 1..122: mol_type = genomic DNA
cttccggcggcgtgacctgaccgcaagaggccaatggagtgtgggagctgaaagggtcttcgctggcggccggtaactggcggcggttgggaacggccgagtgtggctcttctggtgtttca

SEQ ID NO: 136 - DNA, 122 bp, Homo sapiens
  source 1..122: mol_type = genomic DNA
ccgttagggctgggagccggctgggcgcgggggtagtgagggtgcctctcgccgtggctgcgcggcggcctcgtccgagaggccggcgccggggcagtgaccggcccgtgcccagccgccgc

SEQ ID NO: 137 - DNA, 122 bp, Homo sapiens
  source 1..122: mol_type = genomic DNA
gcggttccccatcccagggccaccagggcccccgggcccccccgctgcaccggcgtcatccgccatttgctgggaaaagcgacaagaaggaactagtcagtgtggcctacgcatctggcagc

SEQ ID NO: 138 - DNA, 122 bp, Homo sapiens
  source 1..122: mol_type = genomic DNA
ttcactgggccctctgactgtcccaaggcccccgccgccactccagcgccgtgcagccaccgccgcacagccaccgtggccaccaccatgacgactgcgcgtccccctcgcaggtgcgccag

SEQ ID NO: 139 - DNA, 122 bp, Homo sapiens
  source 1..122: mol_type = genomic DNA
gtctttttccaaaaataaaaaataaaaacatgctttcaataagttctttcccccctctggcgagggctactaaatttgctcagcatttaatacgtaaaattggctaacagtgtctgcacagc

SEQ ID NO: 140 - DNA, 122 bp, Homo sapiens
  source 1..122: mol_type = genomic DNA
cctgccgggccggggcgggcggggccgctggtaaacaggctgggttctggtgacaccgggcggcggcggaaggcggcccgagggtcccgcgcgtcccacaaccctccagtcccgctctcctg

SEQ ID NO: 141 - DNA, 122 bp, Homo sapiens
  source 1..122: mol_type = genomic DNA
ctgaagttcccaggagaggcaaaaaatgaggctgatgaaggtggaagagcctggatcatgcggatctttgaatgccaaactgaaaagctggttttattctctggatgacggggaaccagtag

SEQ ID NO: 142 - DNA, 122 bp, Homo sapiens
  source 1..122: mol_type = genomic DNA
tgtcccatgtcagttagcaagccaccaaagtccataagggatcctgtggggtggaaggtccgcggggcctgcttccctgttgctggtgcaggcggagtgtctgaaggctgcacgcatctggg

SEQ ID NO: 143 - DNA, 122 bp, Homo sapiens
  source 1..122: mol_type = genomic DNA
aacaagggctccaaacctagtttcagagtctgacacacaggaactttcgtatacagcacccggttatacacagctttctccctcgtccgccggattcagtgtctgtcgttattgggttcata

SEQ ID NO: 144 - DNA, 122 bp, Homo sapiens
  source 1..122: mol_type = genomic DNA
gatccctcccatctcacagtacctcacaggtctcttcccccgagcagtgcattgctggagcgaggagaagctcacgaatcagctgcaggtctctgttttgaaaaagcagagatacagaggca

SEQ ID NO: 145 - DNA, 122 bp, Homo sapiens
  source 1..122: mol_type = genomic DNA
tcgggtcacggcccttaacaatagcttactcgggtgactcggcatgtgccaccatcagagcggttggcattcatcattactctcagatgtccctaccaacacaggcttcatcagaggcaggg

SEQ ID NO: 146 - DNA, 122 bp, Homo sapiens
  source 1..122: mol_type = genomic DNA
cgctcagcaaacgctttacttgcacaagctcttcatagcaggcctctgcaaaccagcgggcgccggggagaagggctgcttcttcactagagttggcggcgagggagcccgcttcgaggggg

SEQ ID NO: 147 - DNA, 122 bp, Homo sapiens
  source 1..122: mol_type = genomic DNA
gcgatgggtcccagtcattaactggctgtcaggttcctcagatgatggagctaaaaatagcgcgctatagatagaagcttctcccacgcaggcaggcgccggctgcaaatggaagtgggggg

SEQ ID NO: 148 - DNA, 122 bp, Homo sapiens
  source 1..122: mol_type = genomic DNA
cctcccgggttggccaatgaaaagctggcactgggtcggaggcgccagccaagtggggggcggagcttccaccaccggccaatggggatctggcttcgggatgtgggcggggtccacccggt

SEQ ID NO: 149 - DNA, 122 bp, Homo sapiens
  source 1..122: mol_type = genomic DNA
ctcggtgggtgggagttggtggcctctcgctggtgccatgggactcgcatgttcgccctgcgcccctcggctcttgagcccacaggccgggatcctgcctgccagccgcgtgcgctgccgtt

SEQ ID NO: 150 - DNA, 122 bp, Homo sapiens
  source 1..122: mol_type = genomic DNA
tccctgcctggctgaggtggcagcagggggcgggacgcgcagcgctatggcagagggcagcggggaagtggtcgcagtgtctgcgaccggggctgccaacggcctcaacaatggggcaggcg

SEQ ID NO: 151 - DNA, 122 bp, Homo sapiens
  source 1..122: mol_type = genomic DNA
gcggcccccgactttgcgccccgtagttgagttccgtttatggtctgatttccggcctctcgcctgctcgccccgccgcccgcctgtcccgctccctccctcccggggacccggaggagagg

SEQ ID NO: 152 - DNA, 122 bp, Homo sapiens
  source 1..122: mol_type = genomic DNA
ttgctaaaccgtaacccattgttcccgctgttaactcatggacatgccgcgtttcatccacgctgaacggtaacccgttgttactactgtctttttgttttgttttgttttgtttttttgag

SEQ ID NO: 153 - DNA, 122 bp, Homo sapiens
  source 1..122: mol_type = genomic DNA
cagcgccggccgagggccccagcggagctcggcgggggtgcggggcggttccaggagcctcgccccctgctggggacccagcttgtgccctggcgtcgtggccgccggcaggcagcaaggaa

SEQ ID NO: 154 - DNA, 122 bp, Homo sapiens
  source 1..122: mol_type = genomic DNA
cgcactgccataaggagcttcatccaaccctatgaataagctgttacacttccattttaccgatgagacgctgcaaagttgagtaacacagtcgcagcgctcattggtccattgggtagcca

SEQ ID NO: 155 - DNA, 122 bp, Homo sapiens
  source 1..122: mol_type = genomic DNA
ttatccccatttttcagagtgagggctgaggcctagtgtcttgcccaatgtcacaaatggcgaggtcaaaaatcgacagtctccagagtctgctctcttaaccacttaactattctgcctta

SEQ ID NO: 156 - DNA, 122 bp, Homo sapiens
  source 1..122: mol_type = genomic DNA
taaaaatggcccaacccattacattttcttttaggtagatgggggagctggggggcggggcgggggcagtcagggaacaaacagctgcccttagaaatgacacgccctgtgggcaatggcgg

SEQ ID NO: 157 - DNA, 122 bp, Homo sapiens
  source 1..122: mol_type = genomic DNA
tcgttctgggcctgaggctgtggtagcagcaacacctgctctggcttcacctgcagcagccgccgcaccgcgggctgtaagccggacgccactgcctccccgcacgacgaggccaaggtcgc

SEQ ID NO: 158 - DNA, 122 bp, Homo sapiens
  source 1..122: mol_type = genomic DNA
atgacaaaaaagaaagaggtttctcctcaatctaacggagccattaacatctattaataacgccgacagggtaagtaacggagccgcgctcctcggggtggtcaccgggctgcgtggtcctc

SEQ ID NO: 159 - DNA, 122 bp, Homo sapiens
  source 1..122: mol_type = genomic DNA
cgcgttatagagaactgccccctcgctgccccaataccagcgccggggccgcgagcccgccgctgattgggccgcaccgcccgtgacgttagcccggaccccacccctccggcggcaccgcc

SEQ ID NO: 160 - DNA, 122 bp, Homo sapiens
  source 1..122: mol_type = genomic DNA
ccagtaagtttagtcttgtgaagtccgaacgtttgaataatttactcgctgcaggcaaaccgcctacaactaaatccatcaggcccccgtatccgaatcttccttcacgcgagaagccggcc

SEQ ID NO: 161 - DNA, 122 bp, Homo sapiens
  source 1..122: mol_type = genomic DNA
gctcctcatgtgagaaggaccataggaatctcccgtttcacaggtgggcacaccaaggcccgacaatgggtccaggctgccaagggtggagccgagatgcaaaggggcacctcagagcctgc

SEQ ID NO: 162 - DNA, 122 bp, Homo sapiens
  source 1..122: mol_type = genomic DNA
ggcgtccagcagaggccggtcagggcaagaatgcccgaccctcagggtcctcctcagagtcgctgcgggatcacttaggcgcctccggaaacaacactgtctttgcactggaattttcaaaa

SEQ ID NO: 163 - DNA, 122 bp, Homo sapiens
  source 1..122: mol_type = genomic DNA
aatagagcagctcatgggcgtatttgcgctagtgttgggtgttccgctgtgctgtttttccgtcatggctcgcactaagcaaactgctcggaagtctactggtggcaaggcgccacgcaaac

SEQ ID NO: 164 - DNA, 122 bp, Homo sapiens
  source 1..122: mol_type = genomic DNA
tacagggcttaactcattttatccttaccacaatcctatgaagtaggaacttttataaaacgcattttataaacaaggcacagagaggttaattaacttgccctctggtcacacagctagga

SEQ ID NO: 165 - DNA, 122 bp, Homo sapiens
  source 1..122: mol_type = genomic DNA
agagcccaattaagaacttccagagtttagaaatgacttgggttgattatgtgtgcatgacgtgacctcactagacccagcacgaaagggaagcaggcctgggagccctcccccttgccctc

SEQ ID NO: 166 - DNA, 122 bp, Homo sapiens
  source 1..122: mol_type = genomic DNA
ttagagaagcatcctggagtggttgttgttagtaatactgtctgtggagcacagtagcgtcgcagtagaagttagaccaatcccacctaagtagtcagagaatcttaaaaagtaaagcccag

SEQ ID NO: 167 - DNA, 122 bp, Homo sapiens
  source 1..122: mol_type = genomic DNA
aagacttgggaatccaccatcggagagggaaggagctaggactgttttcccatccgttgacgcttttgtgaccatcacccactagtctggcttctgggcccttgactcttagaatgatttga

SEQ ID NO: 168 - DNA, 122 bp, Homo sapiens
  source 1..122: mol_type = genomic DNA
accccacgcccagggcctcacccacccccaaacggcaggagttcataggacccgcggccacgactgcccgcgagcgcctaccgtgggccacgccccccaacgacccagcagggcaagtgtag

SEQ ID NO: 169 - DNA, 122 bp, Homo sapiens
  source 1..122: mol_type = genomic DNA
ttaaaatcctctctcctgaagttgtgtggtccagccgtttgctgaaggaggaagcaaagccggtagtaactcactacatatttgggcagtggaatgaaccctggaagctgacaaagtcgaag

SEQ ID NO: 170 - DNA, 122 bp, Homo sapiens
  source 1..122: mol_type = genomic DNA
aacccttcctggccccctccatcctaacaaagcctgagtcgaacacgaaaggaagatggtcgctgaagcgaaggggagtcatttgtgtccgttccataaatcaagactgtcgcctttcgaaa

SEQ ID NO: 171 - DNA, 122 bp, Homo sapiens
  source 1..122: mol_type = genomic DNA
ggagaagagaagacgtgcagccagacacctgccgccttgtcaggcctgtgtcgccgcctccgcagcccgaaatcatcctgccctccaaggcaccgccctgatgctccaggtgaaggctgaag

SEQ ID NO: 172 - DNA, 122 bp, Homo sapiens
  source 1..122: mol_type = genomic DNA
gtcaggaagactgacagaggcgggcccagcggcagcgctaagtccagtctgggccgcatacgccgcccgcggccaggagtcagcaggtcatcacgttacagctgcaggggagagaccaagag

SEQ ID NO: 173 - DNA, 122 bp, Homo sapiens
  source 1..122: mol_type = genomic DNA
ggaacctaccttggcagcagattaaagacaacccgccacatttagtctcggccccatgaccgatagtgggttcagttcctccaggggcgggggagcctagtggccccgccccctgactcatg

SEQ ID NO: 174 - DNA, 122 bp, Homo sapiens
  source 1..122: mol_type = genomic DNA
ccttgctccgctccacgaggaggccgccaaccgcagggccgcgacacggacgggaagcaacggacactctcccagcaagacgcgtctagagaaagaccgcgtttcggtgcggggggaattta

SEQ ID NO: 175 - DNA, 122 bp, Homo sapiens
  source 1..122: mol_type = genomic DNA
gcgcctggcccgagtttgtcccgcaggctgcaggcgacaggactgcagggccggcaggagcggggcacacggggacctcaggggatcttggtagccgagggccttcctctgagagctgcaac

SEQ ID NO: 176 - DNA, 122 bp, Homo sapiens
  source 1..122: mol_type = genomic DNA
gtggcgccctgagctgctcagttaccagagccgttggggccgatgatgcaggtgaacctccggaaggggccaatgacctggcggccccgccacgacttgaaattttccacaagcagcagctc

SEQ ID NO: 177 - DNA, 122 bp, Homo sapiens
  source 1..122: mol_type = genomic DNA
tggcaggcggcctgggcctcttcctctcctatgtgtggaagtgggtcaggctctccctcccggggcctgggtttctagctctgggcagcgcccaggccttactcatcctcttgcttatagcc

SEQ ID NO: 178 - DNA, 122 bp, Homo sapiens
  source 1..122: mol_type = genomic DNA
gttggcctcctgggcacaggcgtcggacacctgcaggaggtaggccagggcagcgcggtgcgcgggaccagtcagcctggccagggccccgctctccatggcctcctccacgctgcgggcca

SEQ ID NO: 179 - DNA, 122 bp, Homo sapiens
  source 1..122: mol_type = genomic DNA
ccggctaagtcatgtttaacagcctcagaaattatcttgtctccgcgttctttcttctgccggcgagccaggtaatggtaacagagcgaaactccccagtcggaacttctgggttgcagcag

SEQ ID NO: 180 - DNA, 122 bp, Homo sapiens
  source 1..122: mol_type = genomic DNA
gcatccgggcagacaaagccagaaaagcctagaacaggatgcagagtggtaacattagagcgcaccttgtcatgctggccactgggtggcaggggccggtttcagcgaaggtactcacaccc

SEQ ID NO: 181 - DNA, 122 bp, Homo sapiens
  source 1..122: mol_type = genomic DNA
ttgcagcaaaccacttcaagagggagggaataaagcctgcgcttgtttctctaccttaggcgaaggtgacattttggaatttaacttcatagggatttaaaagaaattctaaactgtcacct

SEQ ID NO: 182 - DNA, 122 bp, Homo sapiens
  source 1..122: mol_type = genomic DNA
accccaggggaccggctgaacgagcgcgtggcctaccaccggctggccgccctgcaacaccgactgggccatggcgagctggcagagcacttctacctcaaggccctgtcgctctgcaactc

SEQ ID NO: 183 - DNA, 122 bp, Homo sapiens
  source 1..122: mol_type = genomic DNA
aaagcagcagcgtctacagtctgcctttatgtccagcgggtgaaagccagaaagcacagacggaatctagccgatagggctccatgctctgcagaaaacatcctgacccgaggcctgcaggc

SEQ ID NO: 184 - DNA, 122 bp, Homo sapiens
  source 1..122: mol_type = genomic DNA
ccccgcgggacgccggtgcccggtctcggtcccagcccagagccgctcgcgcctggacgccggccgccccgtcgaacctttgggtctccgagctccccgcccccgcccccaatcaggaccgg

SEQ ID NO: 185 - DNA, 122 bp, Homo sapiens
  source 1..122: mol_type = genomic DNA
cctgaatgtagcaacagaaagggaacaggaggggcaggggcagagaagcctcccgtcccacgtaaataattacaaacagagcacatgacccctggcggtttctgaacgcgcctggcaacagc

SEQ ID NO: 186 - DNA, 122 bp, Homo sapiens
  source 1..122: mol_type = genomic DNA
attccatattgcaactaacctttaagaagtcagcacctgttagtggaaccgcgactgctccgcagagctgctggtatgagcgcccgtcgccaccccacatcccaggcccagccattctgaca

SEQ ID NO: 187 - DNA, 122 bp, Homo sapiens
  source 1..122: mol_type = genomic DNA
agcgtgcggtgtacctcctccttagcaaagctttctcaatgcctcttaggttagacccgccgcagggatgaaggggttgctggcggattgcaggtgcctgcagcacagggcccagaactaag

SEQ ID NO: 188 - DNA, 122 bp, Homo sapiens
  source 1..122: mol_type = genomic DNA
gcctgtccagacagaagctggggcccaccggaggtagctgcagacgcctgagagcgaggccgaggcccctcaggggtaggtggggggaggctggctggggggatgggcagcggggtggcagg

SEQ ID NO: 189 - DNA, 122 bp, Homo sapiens
  source 1..122: mol_type = genomic DNA
tgttctggcggcaaacccgttgcgaaaaagaacgttcacggcgactactgcacttatatacggttctcccccaccctcgggaaaaaggcggagccagtacacgacatcactttcccagttta

SEQ ID NO: 190 - DNA, 122 bp, Homo sapiens
  source 1..122: mol_type = genomic DNA
gtctcagcctctcagcctggactggacaactgggcttcgggaattcatttaaattctacccgctacacgccttccctggattcagggcggcgtccagtgcattcatcacgcgtgtgctgccg

SEQ ID NO: 191 - DNA, 122 bp, Homo sapiens
  source 1..122: mol_type = genomic DNA
cgtcgctttcgtcgttacttgtctgcaggacggctggtccgggcccaggccctcctccagcgacacccaggcctcgatgtagatgctgggcagcccccaccactgcaccgggcctgtgcccg

SEQ ID NO: 192 - DNA, 122 bp, Homo sapiens
  source 1..122: mol_type = genomic DNA
tgagcgctttaatatatattaaatggtgataaataaggggtccaggcagccggcctgacacggcatttgtcttggagggagcagagattgatatctcgtgggtggcattaaaaactcccgcc

SEQ ID NO: 193 - DNA, 122 bp, Homo sapiens
  source 1..122: mol_type = genomic DNA
ctctgtaggtacaagtcaggataaaggcgttgtttactcctgaggccctcccgctgcgtccgaggcagctgctgctgtagttctgtcagggaaggaaggcgggtagcggtagcagagtttga

SEQ ID NO: 194 - DNA, 122 bp, Homo sapiens
  source 1..122: mol_type = genomic DNA
ccgaggcatgagcgggggaaggtgaccaggacttggaatttcataaacgtccccgtcaggcgtgacgggtcgtcagggctgctatcaaagtcagtccgcccatctaccctcaaacaagccac

SEQ ID NO: 195 - DNA, 122 bp, Homo sapiens
  source 1..122: mol_type = genomic DNA
ggaatgaacatgttggaaataaacgctctcattttgcaggcagataaactgggaatcgtgcgtgtaaagcagcttgctcaaagtcttataactatgaattggaaagtcagattcgagctagg

SEQ ID NO: 196 - DNA, 122 bp, Homo sapiens
  source 1..122: mol_type = genomic DNA
tgagtccgctttccggccaacccctcctctcagtcttaggccccactgcaagcctactcccgctcaccgtacagaacctaagcacaggatcagagatggggacaggttgactcagtcccatg

SEQ ID NO: 197 - DNA, 122 bp, Homo sapiens
  source 1..122: mol_type = genomic DNA
cgcctcgagtgccccctcgcgcccaggggtgggagtacagagccaggctcgccattccatcgtattaggtcagtaagattgacaggcacgatacgtatcaataacattgctgtgcacaacac

SEQ ID NO: 198 - DNA, 122 bp, Homo sapiens
  source 1..122: mol_type = genomic DNA
caaactccaactatattcttggcttccctccctcctctggtggaaggaatgcaaaactgtcgacagttctggctattgctacctgtttgcactgtctgtcccaaaggtctcctcgacttgca

SEQ ID NO: 199 - DNA, 122 bp, Homo sapiens
  source 1..122: mol_type = genomic DNA
gcaacgaaggccgcgagagtcgagtgagggcttgagtctggtgggggcgggagtgtctcccgccgccgcgcttgtgccgccgcttctccacacgtgcactcgggtctctcggctccctcccg

SEQ ID NO: 200 - DNA, 122 bp, Homo sapiens
  source 1..122: mol_type = genomic DNA
tccaaacccgagagccgaaacgcacaggtctcggggctgaactcgcgccaggaacacgcccgaggcaaaccacttgacaaccagcttaggttctcagcagaaaggccgacaggcgggggccg

SEQ ID NO: 201 - DNA, 122 bp, Homo sapiens
  source 1..122: mol_type = genomic DNA
ttttatctgccctcggtacgctgatttccaaaacccagcctcatattctatactccaaagcgcactgccaggtgggccaactccagcccccacaatccgatgccaaggccacttcttgccac

SEQ ID NO: 202 - DNA, 122 bp, Homo sapiens
  source 1..122: mol_type = genomic DNA
gaggtggcgtccctgtccccagccaggcggcagcgcgaagctgcctccccgcggggggagcggaagtggcccagctgctcgagtgacttactagttaaaaagctggggttggagctgccacg

SEQ ID NO: 203 - DNA, 122 bp, Homo sapiens
  source 1..122: mol_type = genomic DNA
tttgctctttaggccaaaataccaaacctagacatcctggctatctctattctttaagatcgttcatgcaactaatgcccatattctgaagacccaggtcatcatgatttgaccaccatctt

SEQ ID NO: 204 - DNA, 122 bp, Homo sapiens
  source 1..122: mol_type = genomic DNA
cccccggcggcgaccccgggaagctgcggcaggagggtcccgacaaccctggggggcaggcgcagcgcggcccgcggggcgtctgctggcatgggacgcccaccgggcactgcagctcccgg

SEQ ID NO: 205 - DNA, 122 bp, Homo sapiens
  source 1..122: mol_type = genomic DNA
ttcccgcgcccagaggcatggatcccaggccctgcattttcccagagaatggcgttggtccggagagggagagacaggagcctgcagtcacacagcagggtgggccaggtcccgtgcagccg

SEQ ID NO: 206 - DNA, 122 bp, Homo sapiens
  source 1..122: mol_type = genomic DNA
ggtccagcaccttctgggtggacttcttcacatccccgtggctccttcgggagaacatcccgcggcaggaagcccgggccccgccggcggggcagtaggcgcctgcgccacgcgaatcaaag

SEQ ID NO: 207 - DNA, 122 bp, Homo sapiens
  source 1..122: mol_type = genomic DNA
ctgcttctgtttcgcggatgtccgggaggtgcagtggctcgaggtcacgctgggcttcatcgtgcccttcgccatcatcggcctgtgctactccctcattgtccgggtgctggtcagggcgc

SEQ ID NO: 208 - DNA, 122 bp, Homo sapiens
  source 1..122: mol_type = genomic DNA
ttcttcgggctggtgctggcactcatcggcctcatcttcctcatggtgctctacctaaaccgccgcggcatccagcgctggatgcgcaacctgcgcgaggcgtgccgggaccagatggaggg

SEQ ID NO: 209 - DNA, 122 bp, Homo sapiens
  source 1..122: mol_type = genomic DNA
cacactgcaaaggcgggtgctttcaaaattcactttttcccacaagcggattcagaaatgcggaggttgcctgccgccttttctaccggacaacattcccgagttattggtgagctggaatg

SEQ ID NO: 210 - DNA, 122 bp, Homo sapiens
  source 1..122: mol_type = genomic DNA
ccgtgctgccccaggcaggttcccccacagaggtgtcctgttgagattcctcccatcagacgccgctcccagagctgtggcccgcagccctcctggggcgcctcctgccctgagctgaggcc

SEQ ID NO: 211 - DNA, 122 bp, Homo sapiens
  source 1..122: mol_type = genomic DNA
ctggcacatagaggtgcctggtacgtgtttgttgaatgaatgaatgaatgagtgaatgagcgaacatgccatttcaccttatatatcttgtgaacctgccaggcccgggcctgatgtcatag

SEQ ID NO: 212 - DNA, 122 bp, Homo sapiens
  source 1..122: mol_type = genomic DNA
atggggattagaaacaattcatgtcaagtgcttggcacctaagaagtgctcaataaatagcgactgtaccacacctcctaggagccctcagcgtactgaattagagttctctataagtctcc

SEQ ID NO: 213 - DNA, 122 bp, Homo sapiens
  source 1..122: mol_type = genomic DNA
ccccagagagctttcatctagaaggtttgactctggccagacaaccagcgagcatcttctcgcaatctgttgcttcttccatggcaaactccagagaattaagaagccaaactcaacatcgc

SEQ ID NO: 214 - DNA, 122 bp, Homo sapiens
  source 1..122: mol_type = genomic DNA
ggatctgattattgaggtgtggaaggaataaataatcagtccacaaataaacaaactgtccgggattcctagagggaaggagaaatccttgaaggagatccaagtcgctccaggtctgcctg

SEQ ID NO: 215 - DNA, 122 bp, Homo sapiens
  source 1..122: mol_type = genomic DNA
cagcgtctccaccttgctcagcttcttgctggcgccgccgtgcggcacgtgctgccgcagcgcctggaagcccaagttcaccagcttcacgcggttgcgctcgcgctcattgcgccgcgcta

SEQ ID NO: 216 - DNA, 122 bp, Homo sapiens
  source 1..122: mol_type = genomic DNA
tgcttgaggcggtgctacaaatgaagtccttctccaaatgcattgagccccaagaaaaatcgctgattctgaggaaccttccaactttaacaatttcaggacgtccctaagctgagctagga

SEQ ID NO: 217 - DNA, 122 bp, Homo sapiens
  source 1..122: mol_type = genomic DNA
gggccaaacaccgctcagcctggtacccctaagacctcactcaaaaagtcagggagttttcggccacttcagaaagactttgttgttacccctccgtcttaacccaggtgagttaagtgaca

SEQ ID NO: 218 - DNA, 122 bp, Homo sapiens
  source 1..122: mol_type = genomic DNA
cgtcgaagagaacggccctcgggcgcgtcgcggccgcggctccagagcccctgggcctcgcgctctgagagcctgagccgcttgaggaggccaagcgcccacgaaccgcattccctgccttg

SEQ ID NO: 219 - DNA, 122 bp, Homo sapiens
  source 1..122: mol_type = genomic DNA
tctgcagaacagccaggaaacaagcccggcccatggcgccacctgctgcctgcctgcctccgctcatgcaccctgggctgggatggtacttctgttcgtctggcattattgcccttggtcat

SEQ ID NO: 220 - DNA, 122 bp, Homo sapiens
  source 1..122: mol_type = genomic DNA
aaagacagccttgactcaagcatgcgttagagcacgtgtcagggccgaccgtgctggcggcgacttcaccgcagtcggctcccagggagaaagcctggcgagtgaggcgcgaaaccggaggg

SEQ ID NO: 221 - DNA, 122 bp, Homo sapiens
  source 1..122: mol_type = genomic DNA
gaggcgggcagcgggggccgcgagcgggcggactggcggcggcggcagctgcgcaaagtgcggagtgtggagctggaccagctgcctgagcagccgctcttccttgccgcctcaccgccggc

SEQ ID NO: 222 - DNA, 122 bp, Homo sapiens
  source 1..122: mol_type = genomic DNA
ccccagccagcccatgctgccccgaggcgggagccatccctttccccagccccagctctgcggccactcacctgctccgagaccgaagatcaaacagaatgttctcagtaagacccgagact

SEQ ID NO: 223 - DNA, 122 bp, Homo sapiens
  source 1..122: mol_type = genomic DNA
tatttgagctcaaaccaagcgactgttgactttagcacacaaagcaaagatttcactgcccgctagtttaaaaatgaatattttaccaagatatcgatcagcgttataaaattcagttaagt

SEQ ID NO: 224 - DNA, 122 bp, Homo sapiens
  source 1..122: mol_type = genomic DNA
gcagtgagctgagattgtgccactgtactccagcctggcaacgcagaggttgcaatgaaccgacacggtgccactgcactccagcctgggcgacagagtgagactctctcaaaaaaaaaaaa

SEQ ID NO: 225 - DNA, 122 bp, Homo sapiens
  source 1..122: mol_type = genomic DNA
cgcctaccgccctagagcaggaaatagcggtcagcgccagctgtgaggagcacagcattgcggccaacacaggaggcactgaccacggggcaggcgctatttaaaaatcgtggcaaaggatc

SEQ ID NO: 226 - DNA, 122 bp, Homo sapiens
  source 1..122: mol_type = genomic DNA
gcagcctcctgggaacacagcgtcccattcccaggggctcagcgggctggcgggagggggcggcgggggccgtgggttttgtttggcggccgggccgttaggattcccagcgccgggcggct

SEQ ID NO: 227 - DNA, 122 bp, Homo sapiens
  source 1..122: mol_type = genomic DNA
gttcgcctacgatggcaaggattacatcgccctgaacgaggacctgcactcctggaccgccgcgaacacagcggctcagatctcccagcacaagtgggaagcggacaaatactcagagcagg

SEQ ID NO: 228 - DNA, 122 bp, Homo sapiens
  source 1..122: mol_type = genomic DNA
ctcgctgcttctcccctagtcttcgggtcccttgaacgcaggtcgcttgtttgccttacgcgtagtcagcggccagtggctatttatggcagtaaggaatattatccacatttcacatggag

SEQ ID NO: 229 - DNA, 122 bp, Homo sapiens
  source 1..122: mol_type = genomic DNA
gcaatgcaggggagctggaaataggcagaagctagatcaggtggtgactgacgtggcagtcgactaaaggagtttcttcaggtttctttgtaaaaggcaacggcaagcctttgaggggtttt

SEQ ID NO: 230 - DNA, 122 bp, Homo sapiens
  source 1..122: mol_type = genomic DNA
cggaatgtgccatttggaccggtcggcagcagctacggttgccggtcccgcactgaaaaacgacagtggtgacgggtgagctcccagaagcagaagaatgacaggcaacacctgaagccacg

SEQ ID NO: 231 - DNA, 122 bp, Homo sapiens
  source 1..122: mol_type = genomic DNA
tcgcgcctgccctgcgcgctgtggttgcggacgccccgaacccggaagcgcggtcccgcgcgcggctcgcccccagctttgaccatatatagtcaagcgctcggctcggcggctgcggtccc

SEQ ID NO: 232 - DNA, 122 bp, Homo sapiens
  source 1..122: mol_type = genomic DNA
ggccgtcttcctcctctttcctttcaccctagcctgaccggaagcagaaaatgaccaaatcggtatttttttttaatgaaatattattgctggaggcgtcccaggcaagcctggctgtagta

SEQ ID NO: 233 - DNA, 122 bp, Homo sapiens
  source 1..122: mol_type = genomic DNA
ctccacgtggtaggtggtgccgggcctgaggtcgggcaggctgacggtgcgcgtggtgcccggcacagtcagctcaccgccggggccctctgcaggcggctgaggccgccagcgcagcacca

SEQ ID NO: 234 - DNA, 122 bp, Homo sapiens
  source 1..122: mol_type = genomic DNA
cacacacacacacaaggctcctccgcaggggactcgggtgggagatctgcaggtgggtgccgtggggagggacagctgcctgcttgtaaatccgccccctgccttcttctgggctgcctctc

SEQ ID NO: 235 - DNA, 122 bp, Homo sapiens
  source 1..122: mol_type = genomic DNA
gcagtggctcaaaggaccgagcgggcggtgcaggttggaacccgcggggcggaccaatcgcggctcggccacagcctcgcccgctgattggtccctccaggccccgcccccgctcgccccgc

SEQ ID NO: 236 - DNA, 122 bp, Homo sapiens
  source 1..122: mol_type = genomic DNA
ctccacacctgccccatctgcagcaggtggatgaccgactgccagatccacacggagaggcgacacaggcgctgcgcccccggcgtgggggagaccctcacgcctgggccaccgcgggccgc

SEQ ID NO: 237 - DNA, 122 bp, Homo sapiens
  source 1..122: mol_type = genomic DNA
ggtgggactggggtgcagaacctaagatctggctattgtgttcatggctatgtggtgacccgcttcacctcacagaagaaagagctgactccccaaagaggggctggaggtccccctagtcc

SEQ ID NO: 238 - DNA, 122 bp, Homo sapiens
  source 1..122: mol_type = genomic DNA
tccgactctacggacccaggtcgctgtggcccatcgctttcgatttgacttggtttctgtcgccactcgcggaaggcgcgccccccgccctcgctcggcggcccgccccgccccgcccctgc

SEQ ID NO: 239 - DNA, 122 bp, Homo sapiens
  source 1..122: mol_type = genomic DNA
tgcccaagagcgctacgtcgccggcgggcagcagcagcgcctacaaactggaggcggcggcgcaggcgcacggcaaggccaagccgctgagccgctctctcaaagagttcccgcgtgcgccg

SEQ ID NO: 240 - DNA, 122 bp, Homo sapiens
  source 1..122: mol_type = genomic DNA
gcggtcgccgccgcggcgggcggtgggccgggttctcctttgaaggggcggtgggaccggcggactctctgggcactggctaccacggagacgccgctacgcttcggggcggggcccgtctt

SEQ ID NO: 241 - DNA, 122 bp, Homo sapiens
  source 1..122: mol_type = genomic DNA
cccttaaaagctggggcctgggacaggaacgacagacaatgcagccaatggcgtcacgcgcggtgccccgctacccaatcgaaaggcgtggctgagggaaacgcggtgggaaccgcccccga

SEQ ID NO: 242 - DNA, 122 bp, Homo sapiens
  source 1..122: mol_type = genomic DNA
tcctcctccctcttcctgggggtgctgctacttccccggccttgtgtgcaggactggggccgccgttacctttcctcgacccaccagacccctcaaccacacaacccgagacgaattcccgc

SEQ ID NO: 243 - DNA, 122 bp, Homo sapiens
  source 1..122: mol_type = genomic DNA
ggaactccgctggtgggagtaggtgtcttctgtgcattttttttccaaaaccactttggccgttagatggctgtgggccggcactccatccatccatccatccatccatccacccacccacc

SEQ ID NO: 244 - DNA, 122 bp, Homo sapiens
  source 1..122: mol_type = genomic DNA
ccgggagctgggttataaaatgccgggttaagcggcaactcagactcaggatcccgctcacgacatggcctcgggcgctcagctcccgccgcagccgtcgagctcagaggtcagcgccgtcc

SEQ ID NO: 245 - DNA, 122 bp, Homo sapiens
  source 1..122: mol_type = genomic DNA
tgggggcccagggggtgtcagctcggggccttgcctcttgcagctactctgtggtcaggccgggtcctccaccatcaggaagatcccatcctgagctctgtctcctgcccctcctgctgtgg

SEQ ID NO: 246 - DNA, 122 bp, Homo sapiens
  source 1..122: mol_type = genomic DNA
ggtcttgtgtgttcagaggctggttttacaggtgaagagaagaaacagccgcagaagttgcgattgtccaaggtcacttaataagtggcaagaattaggatgttaagtgttctcacccccag

SEQ ID NO: 247 - DNA, 122 bp, Homo sapiens
  source 1..122: mol_type = genomic DNA
cggagattcccaagtcagattcacaaacacatggggcgtcctggtggataaacctttccccggaagacacatttgtgaagagtcttggcccccagtgttgagactgattcggcgtcctgaaa

SEQ ID NO: 248 - DNA, 122 bp, Homo sapiens
  source 1..122: mol_type = genomic DNA
cccagacggcagcctcccgcggacccagcccctaacacaggtgcagcttctggtgctgcgcgaggtgcgttttatagcggaagcctttgccgcagcccgcacacttgtgcggcttgttgccc

SEQ ID NO: 249 - DNA, 122 bp, Homo sapiens
  source 1..122: mol_type = genomic DNA
cgcagcggcgtttcattagagccccgggcccgggccgcgcgccaggaacttccccgcacgcggcgagatcgacgatcccccgcccccagccccagcccggctccaggcctcgcaatgtcagg

SEQ ID NO: 250 - DNA, 122 bp, Homo sapiens
  source 1..122: mol_type = genomic DNA
gtctgcaggcaacattcaactgcaaggcatcggccaatgggaactattgctgggctcgttcgaaagtaaacggtggacggcgcggcccgaggcaggtggcgggagtcagtttaaggctggcg

SEQ ID NO: 251 - DNA, 122 bp, Homo sapiens
  source 1..122: mol_type = genomic DNA
aggtgacaatgacaacaaaattgacgcggacgctccagtcaaaggcatctcccctttatccgatgactcaccctcttaggaagtcggcccgagaggcaaatctcaaaataccttgacatgaa

SEQ ID NO: 252 - DNA, 122 bp, Homo sapiens
  source 1..122: mol_type = genomic DNA
attaactcatgctcctagcttcgctaggaatgtgatagagaatttcccctgtaggttcttcgtatggtgcgctccgctggatcacgtgagccagttccaaaatggcggcaggggtggccggg

SEQ ID NO: 253 - DNA, 122 bp, Homo sapiens
  source 1..122: mol_type = genomic DNA
tcctgttccacctacgtaggatctgtgaaacaggctcagtgcctttgagggaggagggaacgtttagattgagaccaccccactcccgggtgattaaataaatatgtctctcccccacccca

SEQ ID NO: 254 - DNA, 122 bp, Homo sapiens
  source 1..122: mol_type = genomic DNA
aacctcctctttgaagcccacaagatacggtgaggattactgcctttttctgatttccaacgtgggtttttcattaagcaaagaacaattagaaaacccacacataacatagggtattcagt

SEQ ID NO: 255 - DNA, 122 bp, Homo sapiens
  source 1..122: mol_type = genomic DNA
gccacacagggggaaaggaagcagctggagagtccgctgcccacaccaccccgggctccacggcctcacccccaggccgtcacagagctccagtctcccgccacttcagtgcaaccctcgct

SEQ ID NO: 256 - DNA, 122 bp, Homo sapiens
  source 1..122: mol_type = genomic DNA
ctggaggaccttcttacctgggggaacccgtctcacctggaagaccttcacctgggtaatcgccgtggcctcccactacggcgcagccgggtcggctgcccgggcttcaccctaaaataagg

SEQ ID NO: 257 - DNA, 122 bp, Homo sapiens
  source 1..122: mol_type = genomic DNA
ccagggtcacccccgaaccaacaaagcacacacacacccgagatgcgccccaggccgcagcgccaaccccccttcagatgtcccggagacaggcgagcagcggtccccagggctcccgtcgc

SEQ ID NO: 258 - DNA, 122 bp, Homo sapiens
  source 1..122: mol_type = genomic DNA
ggtcaagctcaccctgacgtcagggccttgttgctccctttacatgaactttcttccccacgaggcaatacaggactctctcccacccttgcttcaggtagctgcttgaatttcaccttctg

SEQ ID NO: 259 - DNA, 122 bp, Homo sapiens
  source 1..122: mol_type = genomic DNA
ccggggggatcactgctgttgtcccccacccagatctcctgagggtccggcaggaggtggcggctgcagctctgaggggccccagtggcctggaagcccacctgccctcctccacggcaggt

SEQ ID NO: 260 - DNA, 122 bp, Homo sapiens
  source 1..122: mol_type = genomic DNA
gaaacgcctaccttggcacttagggaccagaagcctctggatgtctagcaacaggggtcacgggatcactgcgtggggtctctgtaagcagtcccctgaggcagtgcaaaaccggaaacctg

SEQ ID NO: 261 - DNA, 122 bp, Homo sapiens
  source 1..122: mol_type = genomic DNA
ggcggcggcggcgccaactgttttcaaacagtggcggacaaacagggcttggggctggcccgcacgctgcctgatcgtttccgcccgccgctccacctccccgcgggccccgcaccccgaga

SEQ ID NO: 262 - DNA, 122 bp, Homo sapiens
  source 1..122: mol_type = genomic DNA
atcaggctgcacattcagcatcgacagccctggccagtaggccttctgggctcccagcaacgcctgcattttgcacattggtttgcatgactggtaatgtcccttcacagggccaccctcac

SEQ ID NO: 263 - DNA, 122 bp, Homo sapiens
  source 1..122: mol_type = genomic DNA
ggaagggtccggagaggggccacaggctcctggcctttctaagcacaccaagtgcccagtcgcggacccccgggaccaggatgcgctgacgacccggctggcaggcgggtcctcgtgggcga

SEQ ID NO: 264 - DNA, 122 bp, Homo sapiens
  source 1..122: mol_type = genomic DNA
agagacctagcgcagagcccaggtggaagttccaggttaccccagacctggcctaggactcggcgctctgagccaccgcagccagtcttttatgcatccggcgggagtttcggtttcctttc

SEQ ID NO: 265 - DNA, 122 bp, Homo sapiens
  source 1..122: mol_type = genomic DNA
tgaaacaaaccgggagggccgtgaggagaccgccgcgtttctcttccgacgcgggtagggcgtgcttgtcccattcccaggaacccaactcatctgaaacaacagggcacaaccgccggcct

SEQ ID NO: 266 - DNA, 122 bp, Homo sapiens
  source 1..122: mol_type = genomic DNA
accaaaaacaataaaccttttatgacttaaccaaggaagcacaaattatctccaaagaggcggaaagcaggccttacaagatccaggaccacccccaaagacagttcaaagaaagcaaagtt

SEQ ID NO: 267 - DNA, 122 bp, Homo sapiens
  source 1..122: mol_type = genomic DNA
gcgagtggtctgcgggcagcagctcccagaggcagccttggaattccagctcggactgggcgggaaggcgcaggcggcccaggtcgccgacacgctcacgcaccctccctgcctggccgcgc

SEQ ID NO: 268 - DNA, 122 bp, Homo sapiens
  source 1..122: mol_type = genomic DNA
tttccctgctgccatgccctttggcaggcagccgtccccacgcccggaaagccccagcttcggctcagcccacagcacaagggcatatccttctgcctgcgcaggccagggtgctcgacgcc

SEQ ID NO: 269 - DNA, 122 bp, Homo sapiens
  source 1..122: mol_type = genomic DNA
gcaggcacctccaggcctggctggcagcgccctgggagtggtcccctggtacctctgcggcggaaagagggtaacaacaggcttcctatctgaggctaacccctaggtcgacgtaccaggtc

SEQ ID NO: 270 - DNA, 122 bp, Homo sapiens
  source 1..122: mol_type = genomic DNA
cttgattctcattgttcccgggagcgagcgccttggctgcgctgggcatacccaccctggcgccattcacaggcagtgcctgccctggccctgtgctcaccccatcaggcctcctcttccgc

SEQ ID NO: 271 - DNA, 122 bp, Homo sapiens
  source 1..122: mol_type = genomic DNA
tggggccggggccatcgccgtggcgtcttgaggacctgacgcgagctctgtggtcctggacgccagcccagggagggcagatgtctgcactgactgggatccggggctgtgagggcggcgct

SEQ ID NO: 272 - DNA, 122 bp, Homo sapiens
  source 1..122: mol_type = genomic DNA
gccttgccgccagtcttgccgcggcccgacatgctagcgaggtagaccggtgaagcacgacggctcaaacactagaacagacgcccgccgcagtgtaactgctgtcgcgcgcgcgccgcgag

SEQ ID NO: 273 - DNA, 122 bp, Homo sapiens
  source 1..122: mol_type = genomic DNA
cgcgggccccgggcccggggatgacgccgcggagacccccggcctgcccccggcccacagcgggaccctcatgatggctttccgggacgtcacggtgcagatcgccaaccagaacatatccg

SEQ ID NO: 274 - DNA, 122 bp, Homo sapiens
  source 1..122: mol_type = genomic DNA
gcagcgactgtggcgcgcggagtccgaactgcagatcccgctgccgccggagccccgccccgcgtggggcgagctccccaagctccgcctccaggctccccggcctctccccacccttaagc

SEQ ID NO: 275 - DNA, 122 bp, Homo sapiens
  source 1..122: mol_type = genomic DNA
ccaaagctccctctacaccaagctgctccctgtacggcaacagaactcctggaacttgtccgcagagaatgagcagggcccttgcttttttatggtcttggccaggtttagcctgccttgta

SEQ ID NO: 276 - DNA, 122 bp, Homo sapiens
  source 1..122: mol_type = genomic DNA
caaagtcactcagctcgccaggggcagagccagggtgctcacagggtggccaaccttccacgtctgccctggacacgggactttcagtactaaaatgtgcggacgtccttctcccggacgtc

SEQ ID NO: 277 - DNA, 122 bp, Homo sapiens
  source 1..122: mol_type = genomic DNA
catcctcgccagtaaccaaagaagcgcaaattagcgcaaaaagcaacgccgctttgctgccggagcagcaaaatattaaacactaaagcccccgtgctggtgaggatttggagaaaccagga

SEQ ID NO: 278 - DNA, 122 bp, Homo sapiens
  source 1..122: mol_type = genomic DNA
accgggcctccgcaggtgcagctgggagccctgtctgggcgtggccctcttttttggggccgcggcatagccttgagtgagacgggtggggtaggcaggtttgggtggggtggggggttctc

SEQ ID NO: 279 - DNA, 122 bp, Homo sapiens
  source 1..122: mol_type = genomic DNA
ccgagggtgagctcgctggcagaggaacttgcgccttccaactcttaggtttgttgcgttcgggtcgcagtcgactttaatgggaaccaagcaaagcacccaatggcccctgccatcaggtg

SEQ ID NO: 280 - DNA, 122 bp, Homo sapiens
  source 1..122: mol_type = genomic DNA
gaggacgtcgaagtgcgcactagccctccatggaagacgggagcagaggacggtggagtgcgcaccagtcctccgtggtagacaggagcagagaatgcctggagtgtgtactagccctccat

SEQ ID NO: 281 - DNA, 122 bp, Homo sapiens
  source 1..122: mol_type = genomic DNA
agcgtgtcccgcttgaggctgctgcccttgttcaccaccttgatcctgagggccagtttccggacgctggcgggccccaggccgtcgaagaagaaatcctcgttgaagacggggcggcggct

SEQ ID NO: 282 - DNA, 122 bp, Homo sapiens
  source 1..122: mol_type = genomic DNA
gggccgctgggctcgcggagctggctttggctggtagctggaagggcaaactggggaacccgccggtgccttggcgcgcaggggcgcggcgaaggatcagcaccgcggacagcgcccaggcc

SEQ ID NO: 283 - DNA, 122 bp, Homo sapiens
  source 1..122: mol_type = genomic DNA
gcgcgctgggcactggcgggcgggaggggaggggaggggcgggcggagccgttaccagggcgcccggccctgccccgggcagtgccactgtccgattccaggatgccgagtggctgccggtg

SEQ ID NO: 284 - DNA, 122 bp, Homo sapiens
  source 1..122: mol_type = genomic DNA
cccgtaaccccagccagcacgacattcagacacccctccaggcccaattagcttcacagacgctcaagacttgggaaaacaaaagaggagaagataactgaacccctctccctgtgcccacc

SEQ ID NO: 285 - DNA, 122 bp, Homo sapiens
  source 1..122: mol_type = genomic DNA
ggaatggttagtgagcccaggcaagaaactcatcagccgcatccttcagaacacactgggcgtcctgaacttaggcttctggggacagacacctaccctcgatattgttattccaacgagcc

SEQ ID NO: 286 - DNA, 122 bp, Homo sapiens
  source 1..122: mol_type = genomic DNA
gcctttccccgcaaaccgcaggtccggcgaggactcgggaccccgaactcacccagctggcgagggagaagacacccagcacagcccccatggtgacgccagtgatggaggtggccggtcct

SEQ ID NO: 287 - DNA, 122 bp, Homo sapiens
  source 1..122: mol_type = genomic DNA
agacaactaaattatgcacaccccactttgttttattagcttattagctactcgcactatcgactgtgtagaagtgcaaacacttctcagccccaaccataaactgcttatttataaataac

SEQ ID NO: 288 - DNA, 122 bp, Homo sapiens
  source 1..122: mol_type = genomic DNA
aactgcactgcccagaaccgggtttcccaacctgtagaggccgcatccgcgtctcctgggcgggcagtgccggtaatccccaacaagccccagcctgcttggaattaacgggtctgactgtg

SEQ ID NO: 289 - DNA, 122 bp, Homo sapiens
  source 1..122: mol_type = genomic DNA
taaaggcagagctgagaagatacacgtgctgcagagggcgtagccctggaataacatgcacgatctctcatccctccagatccatcttcggttgtgaatccggcccggaatgcagccacccc

SEQ ID NO: 290 - DNA, 122 bp, Homo sapiens
  source 1..122: mol_type = genomic DNA
ggcgggcgctgccttccaggggtgaagtgttttcggaccccggaatctgtgggcggcctgcgggaggggctgaggcgcagttccctactcacccaggtccgaatccaccgcggtgctgtttc

SEQ ID NO: 291 - DNA, 122 bp, Homo sapiens
  source 1..122: mol_type = genomic DNA
cacccccattgcccagccacccaggtgagaggctgtcgggaaagctgctcaggcgaagcgcgaccagctcagaacgactctccaaccactgctctcaggcacgggagggaaggcctgggcgg

SEQ ID NO: 292 - DNA, 122 bp, Homo sapiens
  source 1..122: mol_type = genomic DNA
tggggtaggggccttggcagtcactgggactcctaacaccgggccagcaggagactaaggcgcagtagcggggcccagagcatacaagggattgggctttggcttctctgctgcagccctga

SEQ ID NO: 293 - DNA, 122 bp, Homo sapiens
  source 1..122: mol_type = genomic DNA
cactgcactccagcctgggtgacagagtgacactccatctcaaaaaaaaaaattttgaaacggccgcaccctcgccggccctgcgtcgtccccgaaaaccagacgccctggggcgcggggcc

SEQ ID NO: 294 - DNA, 122 bp, Homo sapiens
  source 1..122: mol_type = genomic DNA
tcgtgattatatgtcccaaataacccgtagaaataataactgtcatgaaaggagaagccacgtgctctatttgtccacaggctgaggaccaccttgctgcggttgaccgctgctggccggga

SEQ ID NO: 295 - DNA, 122 bp, Homo sapiens
  source 1..122: mol_type = genomic DNA
cagtgggaggggtgggtggaagaaggctggtctctgtctgaccaagcccccccagaataacgcaggctgcccccctaggtggaaacaatgacacaatcagctcccaataccaagggcctgac

SEQ ID NO: 296 - DNA, 122 bp, Homo sapiens
  source 1..122: mol_type = genomic DNA
ggaccgcccggccttggacccatccggagccacaggttggaggagataagtagctgtccccgtgctcatcgccctgtggagcagatcctgtctccttgctgacggtggagcccgggagttcc

SEQ ID NO: 297 - DNA, 122 bp, Homo sapiens
  source 1..122: mol_type = genomic DNA
gcttaggagagaaattggccacgatgaatacactagaggtaatactttagagttttgctgcgaaaggcggcaaggaaaaaggatagtacctgttagggaaagcagagttcagattcttttag

SEQ ID NO: 298 - DNA, 122 bp, Homo sapiens
  source 1..122: mol_type = genomic DNA
ctatccctgtgacaggaaaaggtacgggccatttggcaaactaaggcacagagcctcaggcggaagctgggaaggcgccgcccggcttgtaccggccgaagggccatccgggtcaggcgcac

SEQ ID NO: 299 - DNA, 122 bp, Homo sapiens
  source 1..122: mol_type = genomic DNA
tttggaaaatgagactaccacttggcaatttggtgtcctcattccactgcatcaaaacaccgagaagcagggccaggcacggtggctcacgcctataatcccaacactttgggaggccaagg

SEQ ID NO: 300 - DNA, 122 bp, Homo sapiens
  source 1..122: mol_type = genomic DNA
taaacttcctgaaaaaaaggatgacaggtaaggattaggcagagattaaatctgagtgatcgatcctattcagttaatgggttggcaagtcctggaatgagatacagccatctaaaaattaa

SEQ ID NO: 301 - DNA, 122 bp, Homo sapiens
  source 1..122: mol_type = genomic DNA
tcactctcagctttcaaaagcagaatactgttctattaggtgtttctctctgcatgttgtcggcagtgttctgaataggaggagatccctgcctttaaagggggcgagattggggtggaggg

SEQ ID NO: 302 - DNA, 122 bp, Homo sapiens
  source 1..122: mol_type = genomic DNA
ctgccggccctggggcattgagcctcaggaggccctcgggctcaaggggccctcctggtgcgctcttcttcccaggggagcgggactacggcccccccattgacctgtggggtgctgggtgc

SEQ ID NO: 303 - DNA, 122 bp, Homo sapiens
  source 1..122: mol_type = genomic DNA
tattcagagccaggcagcaggagggagctttgccccagaggaagctcagccatgctgttacggagagggcgcgctcccctcgatgcaccagccgttgtcagaggaggcccacggcagcgggc

SEQ ID NO: 304 - DNA, 122 bp, Homo sapiens
  source 1..122: mol_type = genomic DNA
cctggcctgcccggcccgcgtggtgtcccagtggctgcggccacgccaggcattctgccccgcggcggctgcacagggacgagaactgagaacccctgctcaaccccatccggggtgactgc

SEQ ID NO: 305 - DNA, 122 bp, Homo sapiens
  source 1..122: mol_type = genomic DNA
cagtcccgggaacacacttgcataacctttggtaattggaaatatatctcatattggccacgtgcacaataattcagtgtgaatatggccaataaacatgccttgtttacaggtcattagtt

SEQ ID NO: 306 - DNA, 122 bp, Homo sapiens
  source 1..122: mol_type = genomic DNA
cacaggttagcggcagacttgatcccgagtctcctaactggcaacccaagactctatccccggaactggcaagaatcttcctgaactaccccgataaaattttgagtgccaaagaaagtccc

SEQ ID NO: 307 - DNA, 122 bp, Homo sapiens
  source 1..122: mol_type = genomic DNA
aatagtggaaaggaaagtcgtggagaaggccagcacctgcccggtgtggggagcagggcccgggcacgtgaacctttccctgcggagctggtgcctgtgggtgcacgggtgtggtgcgtttt

SEQ ID NO: 308 - DNA, 122 bp, Homo sapiens
  source 1..122: mol_type = genomic DNA
gccgctagccgtaccccaaagtgggcagaagcccatgaggggaaggtgaggcacctggggcggagagaaaaggaaaaaaccttgccacggagaagggaggcctgggttccccatgaaagaaa

SEQ ID NO: 309 - DNA, 122 bp, Homo sapiens
  source 1..122: mol_type = genomic DNA
ttagacacagtgggggttgaatggatgcgcgcgatataagcaaattaaatggttcgtggccgcatcctagagccctttaattagagttaccatttgtagaaggcctgccgtgcacaatccct

SEQ ID NO: 310 - DNA, 122 bp, Homo sapiens
  source 1..122: mol_type = genomic DNA
aatgttttccacaccgcgtgtgaaatgacgagagcttttgtgtcacacatgtagctgctacgagacagacgctcttgttccggatcagagcagcagacagaaccggcacttttagggtccaa

SEQ ID NO: 311 - DNA, 122 bp, Homo sapiens
  source 1..122: mol_type = genomic DNA
aaatatgagtttggtaagtcctgagctcccgtgataaggacttggtactggtgaggatgccgatgttgagttgcatgacagctgtttccactctgcttaaaaacactcagctccctcctaag

SEQ ID NO: 312 - DNA, 122 bp, Homo sapiens
  source 1..122: mol_type = genomic DNA
tcaattgttttgtgaagggaaaaaaatctcaatttgctttggaaggctgggagacaataccgcaatttgggggctccaggaaaatcgtttttgaatctaaagatctagggaagttgctcaga

SEQ ID NO: 313 - DNA, 122 bp, Homo sapiens
  source 1..122: mol_type = genomic DNA
ttggctggccctctgatatgaggtggtggtgaattggtgggcgtcagtgcagtaagtgagcgagccggacgtcgattgactggtcttcagaagcaggttcggttttgttttgttttggtttg

SEQ ID NO: 314 - DNA, 122 bp, Homo sapiens
  source 1..122: mol_type = genomic DNA
cctccccctccccccgaggcagctgattggctgttgaaagttccgcccctcctaattggccgtggcccgtgccgtggcccgccccttgctgtgtctcctctgattggttgcctttgaggctg

SEQ ID NO: 315 - DNA, 122 bp, Homo sapiens
  source 1..122: mol_type = genomic DNA
aggaaggaaggaaggaaggaaggaaggaaggaaggaaggaaggacacacgtaaatcccagcgattttatttaattttgagacggagtctcgctctgtcttgcccaggctggagtgcggtggc

SEQ ID NO: 316 - DNA, 122 bp, Homo sapiens
  source 1..122: mol_type = genomic DNA
ggaaaacaataatggccagttacccacaattggggcgggggcaggggaaggtgacggaaacggctagttacccagaattctctgggggaaccagaaaaatcggttatctagaattctcccac

SEQ ID NO: 317 - DNA, 122 bp, Homo sapiens
  source 1..122: mol_type = genomic DNA
gaattcatgtcaagctatccctcctcggtggagactcagtttctccacttagggaacccgcgggatggaggtggggagcaaaagcgtcggcgcgccccctcccccttcgcagactgggcccc

SEQ ID NO: 318 - DNA, 122 bp, Homo sapiens
  source 1..122: mol_type = genomic DNA
gcgagcgctcgggggctcaaactgcctggagacaggcggggctcctgctgaggcttggtccgcccggacgagtaaggagaggagccagggaggacctgctggcgtgagacgcctctgcccgg

SEQ ID NO: 319 - DNA, 122 bp, Homo sapiens
  source 1..122: mol_type = genomic DNA
tagatcttatattacataccaactcggctattcatttaagggggtgatagccccttttcccgcccctctgcacaaagcttgcaggggagcttataatgaaggaatgaagcaaagtgtcccat

SEQ ID NO: 320 - DNA, 122 bp, Homo sapiens
  source 1..122: mol_type = genomic DNA
tcctacccccagggctggcagggcagagagagctgatgaaattggcttgggtggagttcccgcggttacagataagaccttggggacaggtagtcctcagtccccaaatgcttacgcagcgt

SEQ ID NO: 321 - DNA, 122 bp, Homo sapiens
  source 1..122: mol_type = genomic DNA
cgcctggggtggagcgggtgtgttccgccgggctccgggatgcacttgcgcagtttcacccgaggctggagtgacgccaacctgttaatgttcgttttcggattctggacttcgggttccgc

SEQ ID NO: 322 - DNA, 122 bp, Homo sapiens
  source 1..122: mol_type = genomic DNA
cggcgcccccgcgaggccggacggggaactgcggccgacgggccctcggcatggcctcgccgccgccgctctggccctggccctggcccggaccgcccactacgcctgatgccgggatggga

SEQ ID NO: 323 - DNA, 122 bp, Homo sapiens
  source 1..122: mol_type = genomic DNA
cacctcggcccccgacggagccggcacagagcaagaatgaggtggtgctcatcccacacacgacgttccctgtgctggcgtcgaagcacctgcacaacgttgcttctttgggaacacgcatt

SEQ ID NO: 324 - DNA, 122 bp, Homo sapiens
  source 1..122: mol_type = genomic DNA
ctcggcctcctcgtcagtgacagatctgtactgcacccctcacagcagtaggtcagacctcgtcctgcccggcacggccggggacttcagcctgagcgccagcctgtcggcctgtacgctgc

SEQ ID NO: 325 - DNA, 122 bp, Homo sapiens
  source 1..122: mol_type = genomic DNA
cacatgtaccctagaacttaaagtataataataattaaaaagaaaaaagaaaaagagaaacgagggtcttgctctattgcccaggttggactcctgggctgaagtgatccctctccttctca

SEQ ID NO: 326 - DNA, 122 bp, Homo sapiens
  source 1..122: mol_type = genomic DNA
aacacagtatctgtgacacagtaagtgctcaataaatatcagcttttatttttcaaggggcgaggaaggtgtattttgaaaaagcatatactatttatttttttaaaggcctacagaaagta

SEQ ID NO: 327 - DNA, 122 bp, Homo sapiens
  source 1..122: mol_type = genomic DNA
cctgggcccctctcccgctgcccgggatctcttctgtccaggtcaccccaggctggtcaccgtccgcccctgtctgtgcccattctcaaggcctcgtgtctgccctccccgcgcggaggtga

SEQ ID NO: 328 - DNA, 122 bp, Homo sapiens
  source 1..122: mol_type = genomic DNA
aggatccagcgcagtctacagggaagctgctttgctttcgatgctgccagcacggccaggcgcctcattcccagctctcgctcccacctctctgagccacagaccccgtgtgggtgaggcgg

SEQ ID NO: 329 - DNA, 122 bp, Homo sapiens
  source 1..122: mol_type = genomic DNA
gtctgtctgccctcccctagcttcaagtttctggagcccctcccttaactttcatgtctccgagatttctcatttcgaattctcaggctcgtctgacacatttccacttctttattcctact

SEQ ID NO: 330 - DNA, 122 bp, Homo sapiens
  source 1..122: mol_type = genomic DNA
gggagagagagagagaagggaaaagagactagatggagatggagaggctgagaaaaagggcgtgagtgacagaaaatgcattcatcctgtctgctgtagcagaggccagaagtagcagccgg

SEQ ID NO: 331 - DNA, 122 bp, Homo sapiens
  source 1..122: mol_type = genomic DNA
ttataagtttaaagtaacaagaaggaccctgagctcccacggagatgattctggaagcagcgatgcccccgggttggagcagaggagcagcgagggttttctgcgctcggcatcgcggcggg

SEQ ID NO: 332 - DNA, 122 bp, Homo sapiens
  source 1..122: mol_type = genomic DNA
gatcacaccctttacggacgcggcacctgcgacagggatgcgcgaggagtcagggggcctcgccggatcgaacctaagctggggaagagtatttcttgtatttttaggagaaattctcagcc

SEQ ID NO: 333 - DNA, 122 bp, Homo sapiens
  source 1..122: mol_type = genomic DNA
gcttgcattccaacccagactttgctactttgtagctttgacaagttatttttcctttctcggtcttaatttctctgtgtgtaaagtcaagataacaccaatgtgccttccgggttaaaatg

SEQ ID NO: 334 - DNA, 122 bp, Homo sapiens
  source 1..122: mol_type = genomic DNA
atacgtacgtgttaatggtgctaaataactgatcacctgacacggggggcgggggcggtgcggagagtagaaaggaagagaatgttgatatcgcgcacggagtcctgtgtccctcctttccc

SEQ ID NO: 335 - DNA, 122 bp, Homo sapiens
  source 1..122: mol_type = genomic DNA
ggctgtgcgggaaggggcgggctggctcgggtcctcctgcgagctgcggccggcgcctaacgtgcagaccaggccccaggccgaggctttatcctggaaccaccagtgtgagagacagcttt

SEQ ID NO: 336 - DNA, 122 bp, Homo sapiens
  source 1..122: mol_type = genomic DNA
cggagacgggagcaaaacacagagaatcggggctacaaagccggtgggcaggtttggctacgctcaaaccgggcagtgccgcggtttaggcgtctccttccttcccagcgactgcacaaaat

SEQ ID NO: 337 - DNA, 122 bp, Homo sapiens
  source 1..122: mol_type = genomic DNA
cctgatcagggaacctgggttctataactgcttctactactgatttgtcctgtgacttcgcgcaccaaatttaggcttgtaaattaaactcccagatttctgttttccattttgcagctcta

SEQ ID NO: 338 - DNA, 122 bp, Homo sapiens
  source 1..122: mol_type = genomic DNA
ctcaagtgatccgcccgcctcagcctccgaaagtgctgggattacagacgtgagccatcgcgcacggcttagtcccaagattttagagccaagaagcgtttgcaggaggacgtagggaaaga

SEQ ID NO: 339 - DNA, 122 bp, Homo sapiens
  source 1..122: mol_type = genomic DNA
atgcagatcagtcaggttgacagctcacattttcttgcaaaactcttaggaattttcctccgcaaatccctccgcagtcagtgataagtactaaatgccacaaagaaaacaatttcccattg

SEQ ID NO: 340 - DNA, 122 bp, Homo sapiens
  source 1..122: mol_type = genomic DNA
ggttgccccaggactattttgcatcccacaaggtacccacacaaggtgcctagcatagcacgctacatacactcaacaggtgctcagtatatgtattgcactaggtcagctgaaagctctca

SEQ ID NO: 341 - DNA, 122 bp, Homo sapiens
  source 1..122: mol_type = genomic DNA
gcaggatgacgatggccgccaggtgggacaggtccccagtcagccggaaaatgttcatggcggcggcggcggtggcggtcggcgcagcgcggcggccccggggctgggcggctcaggaggcg

SEQ ID NO: 342 - DNA, 122 bp, Homo sapiens
  source 1..122: mol_type = genomic DNA
caggcagggaactagcccgaagacccagcgtgggcaggccaacgcccctagcctgattctcgaatcttcgccaaaccctcttacaggccaacagtcggggcctttgtgaagggaaactcgct

SEQ ID NO: 343 - DNA, 122 bp, Homo sapiens
  source 1..122: mol_type = genomic DNA
ccctatgaactctgaccccagctcccacactgtcgcctatcaccggcccactctccgtgccgcgtgtcccttaaaagctggggcctgggacaggaacgacagacaatgcagccaatggcgtc

SEQ ID NO: 344 - DNA, 122 bp, Homo sapiens
  source 1..122: mol_type = genomic DNA
ctgctgcaaaaaaaacaacttttggcgcaaagaatgttgcggccagagagcatccgctgtcgctgacaaaggagtagcaatggcaatgagaaaccgccggcgccacggccgaccgcggcggc

SEQ ID NO: 345 - DNA, 122 bp, Homo sapiens
  source 1..122: mol_type = genomic DNA
aagagcaaccctgggaggccgggggcagcgccaggggcaggcggctcccccttgagccttcgtctgacatgggccgtttgtgaagaagggaagaatgtgttagagggagaaggtggtcaccg

SEQ ID NO: 346 - DNA, 122 bp, Homo sapiens
  source 1..122: mol_type = genomic DNA
ttagagcttggaagttccaagaaagacacagattcgtcccccgctaagccagcacttttacgggatcgctgtggggccggatggcttctagacttccgggtgattccgggagatgggggacg

SEQ ID NO: 347 - DNA, 122 bp, Homo sapiens
  source 1..122: mol_type = genomic DNA
actgttttgggccaagcctggctttcaagagtcccctctagtggagataacctgctacttcggcttctcagttcagcccagagcctccctggccagtgaagattcagaaaacccccttctgc

SEQ ID NO: 348 - DNA, 122 bp, Homo sapiens
  source 1..122: mol_type = genomic DNA
caataaataacattttattaatcacggttagctctgtctcctttactttcctgcaggtatcgcggtatcttatgatgccaatccgtataaatattcaacaccccaacatttatttggagaac

SEQ ID NO: 349 - DNA, 122 bp, Homo sapiens
  source 1..122: mol_type = genomic DNA
ggggcgctggtccaccgactggcccggcagttccccagtagcaccgggggtttcctgccgcgcgcgtcacctggttcagtttagagcctccaaagctgcaaggcaaggcgaagatgggcagt

SEQ ID NO: 350 - DNA, 122 bp, Homo sapiens
  source 1..122: mol_type = genomic DNA
gcaataagattttcatgcaccataaactttcctgagtatctcaaccagttttgttgatgccggggtttgttcaaagctgcagattactgggcctcaccccagacctactctacttaaatata

SEQ ID NO: 351 - DNA, 122 bp, Homo sapiens
  source 1..122: mol_type = genomic DNA
agcgagcaacaggcggcgcaggagctggagccggagaacctagtgtgcttagcggtcgcccgctgggttcctcgcgcgcctggccgcccctcccctagcaacggcccggccccgccccgccg

SEQ ID NO: 352 - DNA, 122 bp, Homo sapiens
  source 1..122: mol_type = genomic DNA
gattttatctatagctcaatccaatatttaatgacaacttcagaatatgatatgtattcccgacacacttgctgacatgttttggatcactaagataatgaatcatttatattttgcgaatt

SEQ ID NO: 353 - DNA, 122 bp, Homo sapiens
  source 1..122: mol_type = genomic DNA
ctccggccaggccttgctgcgctgactgcaacacaggctgaaggctcccctgccttcagtcggccgagtgacctgcacccctcccacaggcccggtgacggctatgactgtgctcccgaaca

SEQ ID NO: 354 - DNA, 122 bp, Homo sapiens
  source 1..122: mol_type = genomic DNA
cagaacaagcgcccgatttcaggggagcccagccgaagggttccacagcgctcagcgagccggctgggagggagcaaactctctaactgcagaaaaaaacaaatcttgattccgctttaagg

SEQ ID NO: 355 - DNA, 122 bp, Homo sapiens
  source 1..122: mol_type = genomic DNA
tctacaaatttagccgtgcaatctaatagcgagcaggccgagacgcccgcgattgaaggccggggtggagaaagtaataagggccctggaaattacccccatcccatgtcacccactctttc

SEQ ID NO: 356 - DNA, 122 bp, Homo sapiens
  source 1..122: mol_type = genomic DNA
cgccgcccgacagccacgcagaacagacgcggcagtgcgacgcctcccccactggggacacgagacagcgacagccacgcggtgagccggtacaaggccctctaggcttcagcgggtctgga

SEQ ID NO: 357 - DNA, 122 bp, Homo sapiens
  source 1..122: mol_type = genomic DNA
gccgagcggggggcggggctgcggtgcctgcagaaccttggacagaagctccctagctgccgccgccgccgccgccgccgtcgccgccgccgagcgcgagcccagccgatccccgccgagcg

SEQ ID NO: 358 - DNA, 122 bp, Homo sapiens
  source 1..122: mol_type = genomic DNA
ctatcaccggcccactctccgtgccgcgtgtcccttaaaagctggggcctgggacaggaacgacagacaatgcagccaatggcgtcacgcgcggtgccccgctacccaatcgaaaggcgtgg

SEQ ID NO: 359 - DNA, 122 bp, Homo sapiens
  source 1..122: mol_type = genomic DNA
gccggccgctgtcagctccctcagcgtccggccgaggcgcggtgtatgctgagccgctgccgcagcgggctgctccacgtcctgggccttagcttcctgctgcagacccgccggccgattct

SEQ ID NO: 360 - DNA, 122 bp, Homo sapiens
  source 1..122: mol_type = genomic DNA
ccggaaggcgggaaggggaaccgctgccgggggtcagtcaggtcgttaccctccgtcagtcgcgggctgcccggctccctgcttctctcggcggcgcccatgtccagctcccggacgggaga

SEQ ID NO: 361 - DNA, 122 bp, Homo sapiens
  source 1..122: mol_type = genomic DNA
cgagacccagaagggtgagagggggaagccgcaccctcttcgtgggtgcgatgctgtgaacgtcttccttcaaggaaagagctgcttctggattttccttgaactccaggaggtgctaagaa

SEQ ID NO: 362 - DNA, 122 bp, Homo sapiens
  source 1..122: mol_type = genomic DNA
gggcaatccagggccctcctcgagggaagcggggtttgcgccagggtccccagggctgtgcgaacaccggggagctgttttttggagaaggctctaggctgaccgtactgggtaaggaggcg

SEQ ID NO: 363 - DNA, 122 bp, Homo sapiens
  source 1..122: mol_type = genomic DNA
aggcctccagccactcccatccatctttctgtccctctcaaagtcacttgcctgaccctgcggatgacaaatccgtccacagtcagccatgtgtctgtgcattcgtcagccactgggtcatc

SEQ ID NO: 364 - DNA, 122 bp, Homo sapiens
  source 1..122: mol_type = genomic DNA
atcaggtaaatcagccctggataaaatagcaggaacctgttccgccgaaaccaaatgagtcgtcaggtaaatcagcccttgataaaataacagcacttgttgaccagaagccatgggaccag

SEQ ID NO: 365 - DNA, 122 bp, Homo sapiens
  source 1..122: mol_type = genomic DNA
ttgtcccattttacaaatggcgagttctctgtttctagtcagtaaataatcaagaggagccgggatcaagaacccagtccacctagctccagaggcaatgttcttatggcttcagtgacatg

SEQ ID NO: 366 - DNA, 122 bp, Homo sapiens
  source 1..122: mol_type = genomic DNA
gaggccaccctcagttggttgtcatgtggccctctccatattctaactcacaagatgctccgacctcccgcctgtgcgccccagtggcgcgaaccggagtgcgcctgcgcgcgggccacaaa

SEQ ID NO: 367 - DNA, 122 bp, Homo sapiens
  source 1..122: mol_type = genomic DNA
tgggcaccacacgtgaggagagagcaaagatcccgacctgggaggcccagcgaggccagccgtccccgctggactcgtcagctgctcggccccgcccacaggctggctgccccgccccgcct

SEQ ID NO: 368 - DNA, 122 bp, Homo sapiens
  source 1..122: mol_type = genomic DNA
aaccaccaccgccgccgccgccgccgccgccgcccgcaacccgcctctccctacgggtcccgactgggcaccacttccggtccgacacggccacgtgttacatctaaatggcaccgtccccc

SEQ ID NO: 369 - DNA, 122 bp, Homo sapiens
  source 1..122: mol_type = genomic DNA
tgtcaggttgggggagaggcccaggttcctcacctggcctctgtgaacacttgagggagccgtgctccttgttattgctaggtgtggatgggggttcaggcttcccactaggtctctgctga

SEQ ID NO: 370 - DNA, 122 bp, Homo sapiens
  source 1..122: mol_type = genomic DNA
agaagagggagtactgtccaaaggattagcagacacgcactatgatcactagaaagcaatcgtggtctgtatgtaaggcaatccccaaaataatttgaaaattgctgccacagcctcccaca

SEQ ID NO: 371 - DNA, 122 bp, Homo sapiens
  source 1..122: mol_type = genomic DNA
acctcccccactgtctgctcccccagctctaacaatgcaaccatgatttctaagactgtacgagtttaaaataaggattcttggcagatctgacgaccacaaaccttctgtcattgatgatg

SEQ ID NO: 372 - DNA, 122 bp, Homo sapiens
  source 1..122: mol_type = genomic DNA
atgccagcctttcccaggcacctgccacatgcagggcaggtggacagaccagcaatgacacggtacagtgaggcaagtctttccacccaatcccacaaaacataagccaatgctgggaccct

SEQ ID NO: 373 - DNA, 122 bp, Homo sapiens
  source 1..122: mol_type = genomic DNA
ttcaacagttaggacaatgtgaactgtagctcagctctgctaggtaccttcacaagacctcgaatgctgccccttactatgcctcggttttcttatctataaaaacggcatttttatcttgt

SEQ ID NO: 374 - DNA, 122 bp, Homo sapiens
  source 1..122: mol_type = genomic DNA
gaaactccctctcgcgggatgatgcctttggaagttcagggtttttctctccaccggactcgtctgccctcggggccaaatccgcgaagcgaggaggagctcccaccacacagcctgctgtc

SEQ ID NO: 375 - DNA, 122 bp, Homo sapiens
  source 1..122: mol_type = genomic DNA
gacaaacaaaaggaaaggcggaaacgcagaaacgcagaggtagccagtggatgaggcatgcgcagagcccacgctcaaagcctggcgctgttggcgcccaacctggaaactacctttcccgt

SEQ ID NO: 376 - DNA, 122 bp, Homo sapiens
  source 1..122: mol_type = genomic DNA
ctgggggccgccgcctttggccctggctccgggcccgtgtggctggacgaggtggggtgccggggcagcgaggcgtccctgtggggctgccctgcggagcggtggggacgcggagaccgcgc

SEQ ID NO: 377 - DNA, 122 bp, Homo sapiens
  source 1..122: mol_type = genomic DNA
ctccgctctagcaacgcggccaccatctccatcggcggctcaggggaactgcagcgccagcgggtcatggaggccgtgcacttccgcgtgcgccacaccatcaccatccccaaccgcggcgg

SEQ ID NO: 378 - DNA, 122 bp, Homo sapiens
  source 1..122: mol_type = genomic DNA
gaaccacgtgccggtaggaggtggccaggtagtcgaagtagttgatgttgagtttccgggcgatgtaacggcccaagtattccatttgctgtgggagcaggtggcgctcaactggggccggg

SEQ ID NO: 379 - DNA, 122 bp, Homo sapiens
  source 1..122: mol_type = genomic DNA
agggcctgtgttgctcatttaaatgcaatttgacgtatgtttgggtcagtggcctcagtccggaggctcttacagtaacttggcactgtggaaatcccagaacttcggccagggtctggagg

SEQ ID NO: 380 - DNA, 122 bp, Homo sapiens
  source 1..122: mol_type = genomic DNA
gtgatgccgcgtgtctgtcgagcacctgctgtgctgggcaccatcgtagttctagggcctcgtcagtgaagcagaaaatccctgctctcgtggagctcacgtgctgggggagacaaacagga

SEQ ID NO: 381 - DNA, 122 bp, Homo sapiens
  source 1..122: mol_type = genomic DNA
gtttcctctttgctctctctgccacgcttgaacaacttgccaaggatgccatctctcttacgtccgccgtggtgctccggattctgactcattcggacgaacgggtcggggtcgcggaacct

SEQ ID NO: 382 - DNA, 122 bp, Homo sapiens
  source 1..122: mol_type = genomic DNA
agagtgtagccgccagtccttccctcatccggaaccttctggtccacatttccttccaagcgttagtcctcggctctccatgcttggctccctagtgtcacacaccgtcctttctgggtggc

SEQ ID NO: 383 - DNA, 122 bp, Homo sapiens
  source 1..122: mol_type = genomic DNA
gggcctgtcagctcactgggccatgcctagtagatccaccctttcccatctacagatgctcgtacttacccaggttccggggcacgcgcggggcagcccccgccttccagagaacggcggct

SEQ ID NO: 384 - DNA, 122 bp, Homo sapiens
  source 1..122: mol_type = genomic DNA
aggggccggcagggggcgcccgagcccgtctctggagcacaaagacccccggcccctgcgcgacccggacgagctcgcattcagccaacgactcctgtgagaaacattccagcaagcactcg

SEQ ID NO: 385 - DNA, 122 bp, Homo sapiens
  source 1..122: mol_type = genomic DNA
ttgctccaggggagccccagagttggtggctggctaacccaaggccccagcggcagcctccgcccggccagctcgccatggcacggggtccacagaccctggtgcaggtgtgggtgggcggc

SEQ ID NO: 386 - DNA, 122 bp, Homo sapiens
  source 1..122: mol_type = genomic DNA
tggactaatgtgaccagagtcggctgtgtttggaaataaacttccaacgctccagactagcgaagcgtcgttaaaaaaccgaaggtaccctgagtggtttttagaaactgaaattctgcagc

SEQ ID NO: 387 - DNA, 122 bp, Homo sapiens
  source 1..122: mol_type = genomic DNA
gcattacaataaatatacaataagcatccacaaggcatgtgtgggagactgtgcttatgccgatggcacgggcaggagtaaggcaggcatcaggggaatgtggatgcacgggaggatgggga

SEQ ID NO: 388 - DNA, 122 bp, Homo sapiens
  source 1..122: mol_type = genomic DNA
taagcatatttacaaaatatacaaggaaaaggacctgattccctggctgggtgagtgaaacgttaattcttcaccttggcacctcaacctacagctgtaataagggccgttatgcccaaata

SEQ ID NO: 389 - DNA, 122 bp, Homo sapiens
  source 1..122: mol_type = genomic DNA
ttagtgttcttctgacctggcctgtgtagtcacagctctgcctgcctgccagcctgggcccgtgtacttccacagctgccccacagcagggcaggcttctttctgcaaacagtctaaaatcg

SEQ ID NO: 390 - DNA, 122 bp, Homo sapiens
  source 1..122: mol_type = genomic DNA
tttagcaggcggtataggcaaattggatactgttttatccgtagtttcctcgtggcctcacggcctaacctggaccatcggacagcggagtgctaccaacctgtcattgatgtcattcattc

SEQ ID NO: 391 - DNA, 122 bp, Homo sapiens
  source 1..122: mol_type = genomic DNA
gcggccctacacggccaccgtgtgccaccacattgagaacgtgctgaaggaggacgctcgcggttccgtggtcctggggcaggtggacgcccagcttgtgccctacatcatcgacctgcagt

SEQ ID NO: 392 - DNA, 122 bp, Homo sapiens
  source 1..122: mol_type = genomic DNA
cccgccggaagccgacatctcgagttctggcagaagcaatttgcgcggcgaggagcggagcggcaggaacccaataagctgcttcgcctcggagctgaagcccgtactcaagatggcggctc

SEQ ID NO: 393 - DNA, 122 bp, Homo sapiens
  source 1..122: mol_type = genomic DNA
gcgcctcccttacccagccccttgccacctttacctcgcccagacattctgaaatcacagcgcctactcagcagaaaatcgggctgcccactgtgattgccagtcgcagaccagggcttata

SEQ ID NO: 394 - DNA, 122 bp, Homo sapiens
  source 1..122: mol_type = genomic DNA
ggtcccagattcctgctctgggaggggccctggaatacaggcttctcccagtgcccgaaacgcccccttttcatcccttttggaactggcttccgtggggattctccgggtcccggtgccaa

SEQ ID NO: 395 - DNA, 122 bp, Homo sapiens
  source 1..122: mol_type = genomic DNA
ggactgaagctcccagcacaggggaggcgcccagcgtggctgcagaggaagggaacgcaccgcaggtgggaggggcccagctgatttctcagcgtcacagtgaaaggcacccgtgatgagac

SEQ ID NO: 396 - DNA, 122 bp, Homo sapiens
  source 1..122: mol_type = genomic DNA
aaaggttggctccacggtcccgccggccgcgcaggtctggctgaactgcttggggtcgcccggctcctctcgattttatgaaaatggcctaattgaggtgtgctcttttctttccttcctct

SEQ ID NO: 397 - DNA, 122 bp, Homo sapiens
  source 1..122: mol_type = genomic DNA
ccctcaggccgtcttgtcagactctgagagcggcgtccagctgagcggctctgagcgcaccgcggatgcttccaacggcagcctgcgggggctctcgggccagcggaccccgtccccaccgc

SEQ ID NO: 398 - DNA, 122 bp, Homo sapiens
  source 1..122: mol_type = genomic DNA
gcggagagagacgccggggcggcgatgcgcttcccacagcagagacgcgtcagagtccaacggggaaaggtggaaaaagacgaaccgtgtaaacaataatcaaaagggacatcgggtggctg

SEQ ID NO: 399 - DNA, 122 bp, Homo sapiens
  source 1..122: mol_type = genomic DNA
acctcctgcttgggttcagccaccttcaaatactgcatcaatggctcgtgcctctgcctgcggggctgggccagcgcgggagaggcaggcggagggttcagggagctggggatctgcggtat

SEQ ID NO: 400 - DNA, 122 bp, Homo sapiens
  source 1..122: mol_type = genomic DNA
aaatgtttaattgtaaagcatgagtctgacctaaaaacaagtgtgcccgcagaagtgaggcggcacgcccgttactcctcacgcaggaagcgcagcaatgaaacaaaacgccgtgcgtttaa

SEQ ID NO: 401 - DNA, 122 bp, Homo sapiens
  source 1..122: mol_type = genomic DNA
ttccaggctggaactgcgaagtttcctgtctgattttccaacaatgtaatttctttctagcggaaggaccctcagaaagcaatcagaggggtgcggagtaaaaataaatcaagttcttgtga

SEQ ID NO: 402 - DNA, 122 bp, Homo sapiens
  source 1..122: mol_type = genomic DNA
gcaggggcagcaaggcgggcttgtggaagtgctgcaccagctccgcggacagcagcaccacgacacagcgggtgctgaggaaaaggcttaggtcctctgccgagaaggaggcctcggggccc

SEQ ID NO: 403 - DNA, 122 bp, Homo sapiens
  source 1..122: mol_type = genomic DNA
ggtcacccaagggtggggaaggtcttggctggaggatgaaggggcctcttgtctctggggcgaggcgatgtcaaggggaatgacaaaccaaaccagtccaaagcaaggggactctgtgtcct

SEQ ID NO: 404 - DNA, 122 bp, Homo sapiens
  source 1..122: mol_type = genomic DNA
gctgaacgtctctctcaggccccgcagctcctcctgcagctgggagtctggccaggacagcgtgcagagagaagaaaacgggatcgctgtgtccgccaccactgcacccaccactcccttgc

SEQ ID NO: 405 - DNA, 122 bp, Homo sapiens
  source 1..122: mol_type = genomic DNA
tccgtagtattgtctctggctttgaacgctgttgagggaggggaatgtttgcactcatcccgcatccttttttggctgctatctttgcggggattgttcaaggagaaatccatcctgactgg

SEQ ID NO: 406 - DNA, 122 bp, Homo sapiens
  source 1..122: mol_type = genomic DNA
aggtgtcatccgaattcaggctcctggggcccgggagggtccgactctacggacccaggtcgctgtggcccatcgctttcgatttgacttggtttctgtcgccactcgcggaaggcgcgccc

SEQ ID NO: 407 - DNA, 122 bp, Homo sapiens
  source 1..122: mol_type = genomic DNA
gccgcccttttcgtggtcccagggcccttccaagaaccggctaaaccaacccaagccgcgcgcacacacacttgtgcacacagggagtgtggagccagattcgataaggcaccccgtgtgcc

SEQ ID NO: 408 - DNA, 122 bp, Homo sapiens
  source 1..122: mol_type = genomic DNA
acgtcccccacccctggatggctctcggtgccgcggagcgggcccccatctccgtgtccccgccccccgcccaacccgaggcggcgatcccggcccccacagtcgctcccccttacctgcgg

SEQ ID NO: 409 - DNA, 122 bp, Homo sapiens
  source 1..122: mol_type = genomic DNA
atcccgaggtcagtccctcccattctggggcacccctggagcagctagctccctgcctagcggtggcaaactccagagccccaaggagcagaacggggattccctgtgcgcaggctcctggt

SEQ ID NO: 410 - DNA, 122 bp, Homo sapiens
  source 1..122: mol_type = genomic DNA
ccgcatgccgcggcctctggtgagctgggtggggatgctcctagtgccccgcctgcgtggcgccccccaggggccacccgccacggcgctcgtggtgtctgacccgcaagggcgcccctagt

SEQ ID NO: 411 - DNA, 122 bp, Homo sapiens
  source 1..122: mol_type = genomic DNA
ataacaataataataatggtagcaagcaacgctctgcagtaggggcttctctcgccatttcgtactgaggaggaaacatacttaagaggttacaaaacttgcaccaaacagataaccctcgg

SEQ ID NO: 412 - DNA, 122 bp, Homo sapiens
  source 1..122: mol_type = genomic DNA
ctcgggctccgtcaggccggccagccgcgtccccggcagggtgcgcagcgtcgagcggtacgtctcatggcgcacgccgcccacgttgatcacgatcttgccgctgtcgccaccgccgccgc

SEQ ID NO: 413 - DNA, 122 bp, Homo sapiens
  source 1..122: mol_type = genomic DNA
ggacaaagccaccacctttcacaaaatgaggccagaccacctgcctccctccagtccctgcggcctggagacggagtcaacattcttatctgtgttggatctgaatgttcctccttgcaaag

SEQ ID NO: 414 - DNA, 122 bp, Homo sapiens
  source 1..122: mol_type = genomic DNA
ctggccgcctaataaaagctcatccccgattggctgccccggcaaatcggagtgtaaagccgccccggattggctgaaacacttcctgagcgattatctttgtgaggctcgggtgagcaaga

SEQ ID NO: 415 - DNA, 122 bp, Homo sapiens
  source 1..122: mol_type = genomic DNA
catgttgtaggtggccctggtccccatgatccatggaacagaggcggccccagctgtctccgcaggattatgcaccgcgcgtcatgagccgagggggacaggacccgtggagcaccactctc

SEQ ID NO: 416 - DNA, 122 bp, Homo sapiens
  source 1..122: mol_type = genomic DNA
gaaaaagtagagaaatgtaatatttctttcctgctgtactcactgtaactgtgagaggatcggctcttttaaccaacagataagaaaggaaatattagctatgaagaaatgtctatcaagtt

SEQ ID NO: 417 - DNA, 122 bp, Homo sapiens
  source 1..122: mol_type = genomic DNA
ggggacttggcaatgccaaggtgtttgctgaggctgtggactctccagcccgggagaggtcgccggacctttgaggggcattggaatcctgggctcctcctctgctgggtggagccgcgaac

SEQ ID NO: 418 - DNA, 122 bp, Homo sapiens
  source 1..122: mol_type = genomic DNA
agcacctggcctgtgcctcgtccaggctctggtcggtgatggccatgatctgctgcaggacgtcgctcgtgtcgaggcgccgcggggcggggggcgatggcgcggggcgcggcggggcggcc

SEQ ID NO: 419 - DNA, 122 bp, Homo sapiens
  source 1..122: mol_type = genomic DNA
cccctgaggcctccacctctgaaatctgcagaacagccaggaaacaagcccggcccatggcgccacctgctgcctgcctgcctccgctcatgcaccctgggctgggatggtacttctgttcg

SEQ ID NO: 420 - DNA, 122 bp, Homo sapiens
  source 1..122: mol_type = genomic DNA
gccaggcgcaggcggtcctcacccagcttcaatgcgctggaggcttggcagagctggtcgcgaggaagtacaagcaggctgccaaatgcttcctgctggcttcgtttgatcactgtgacgtc

SEQ ID NO: 421 - DNA, 122 bp, Homo sapiens
  source 1..122: mol_type = genomic DNA
gctggggatgtgaccggtagccggggttgcagtggcaggagtagtcaggggggcccggcacgcactctccgtggccacagatgttctggttcagtcggcactcatcagtctctgcgggcatg

SEQ ID NO: 422 - DNA, 122 bp, Homo sapiens
  source 1..122: mol_type = genomic DNA
ggaggtgggaccaaggccgaatttagggacccccagcatgagctgcgggtgggtcaaggccgcgaggttggagggcacggggacacagcatagcgaggaaggaacggtagggaccgtggtga

SEQ ID NO: 423 - DNA, 122 bp, Homo sapiens
  source 1..122: mol_type = genomic DNA
accaccaaagcgttctgaccggacagtgtcactggagaaggcggcgcgacatgtccagggcgcagatctgggctctggtgtctggtgtcggagggtttggagctctcgttgctgctaccacg

SEQ ID NO: 424 - DNA, 122 bp, Homo sapiens
  source 1..122: mol_type = genomic DNA
ctaaaccgactcctgtattaaaataatacactattattgtgaagtattatctgacaacaccgttttttgaccttggatttctttagggcaggagccatgtcctacttatgactgtttctatg

SEQ ID NO: 425 - DNA, 122 bp, Homo sapiens
  source 1..122: mol_type = genomic DNA
agaaggcgctgagctccagccacgaggagaatctgaggcgcacacgatggtgtgaggcggcgcgggaggggcctgggtcagagctttgtacaaaacagtgtttgcggaatgacaacagccac

SEQ ID NO: 426 - DNA, 122 bp, Homo sapiens
  source 1..122: mol_type = genomic DNA
cgacctgcctttcagccctgaggccctggtggaccgcaaggaattctgggccgtgtgccgcgtgcccgggcccctgcacagcggcgacatcctgggcctggtggtcaacgccgacggcgagc

SEQ ID NO: 427 - DNA, 122 bp, Homo sapiens
  source 1..122: mol_type = genomic DNA
accgttgagccattggtgtcaagtattttaattctctttaaaatttaaaacctgcaagcgcgggagctcagggacctggccaggaaggcctgagcttccgggtcatcttagcacgccccctc

SEQ ID NO: 428 - DNA, 122 bp, Homo sapiens
  source 1..122: mol_type = genomic DNA
agagaagcaggaaggagggagaggaaagagaagagggagatggactggcctcagccaccccggagtacagggatgtcatcacaccagccctccagcggctgaaagagccagtgagaggcagg

SEQ ID NO: 429 - DNA, 122 bp, Homo sapiens
  source 1..122: mol_type = genomic DNA
ccccaccacccccccggagtacttaagggagttggcggcgctgctgcattcattgcgccgcggcacggcctagcgagtggttcttctgcgctactgctgcgcgaatcggcgaccccagtgcc

SEQ ID NO: 430 - DNA, 122 bp, Homo sapiens
  source 1..122: mol_type = genomic DNA
ccagccacgtgcccagcccgtgggtgcggtttcccagggcggccccggcgccgccgcccacgccgtccacctcctccaagccgctgtggttcaggaaggtcgcgttcatcgccgcgcggcgc

SEQ ID NO: 431 - DNA, 122 bp, Homo sapiens
  source 1..122: mol_type = genomic DNA
tgagtccgaaagaacgggggaaagccgaattatgcaaatgcggatccctgcgatggggctcgggttacggcccccgccggccccttaggtgaggcacccaccggcagcaagcgcgggcgagg

SEQ ID NO: 432 - DNA, 122 bp, Homo sapiens
  source 1..122: mol_type = genomic DNA
gcatggccaccgcaagcgcaactaaaatccagggcttgtcgcaggcacaggctcctccttcggtgggtgggacggcggcgcgcactttctctacgccccactgctaggatgtgcggccacca

SEQ ID NO: 433 - DNA, 122 bp, Homo sapiens
  source 1..122: mol_type = genomic DNA
ctgtgaggagggtagtgggggggcattgatgttcccgtttctgagtgaggagacctatgtcggggccttggtgacctcaagccgaatgctcaaactgctactgctactcctaaggttgtgcc

SEQ ID NO: 434 - DNA, 122 bp, Homo sapiens
  source 1..122: mol_type = genomic DNA
tgacagccgcagacacggcggctcagatcacccagctcaagtgggaggcggcccggggggcggaggttcatcctcacagggataggcacctattagatgtggtgtggttttcctctctactc

SEQ ID NO: 435 - DNA, 122 bp, Homo sapiens
  source 1..122: mol_type = genomic DNA
tcacggaacgccgaacctggccgggcccggttgctgctgcggtgttaggtgagttcaggccgccgccatcgcttctcaagcgcaccagccgcgccccgccccgcgccacacgcagcccgcgc

SEQ ID NO: 436 - DNA, 122 bp, Homo sapiens
  source 1..122: mol_type = genomic DNA
taaatccaacgtgacgcctaccgagctgggtacaacattggtttgtttcataaaacagcccggggtgtttgggactcttactcctttacaactctcatttgactgtccaaagatgcttgcac

SEQ ID NO: 437 - DNA, 122 bp, Homo sapiens
  source 1..122: mol_type = genomic DNA
gccgtgagaacccggggacagcctcccctcttgcggtcctgcagtcccggacccactgggcggatcagaaagtttgcagggagccagggactaggagacagacagacagcgcagggacagag

SEQ ID NO: 438 - DNA, 122 bp, Homo sapiens
  source 1..122: mol_type = genomic DNA
cctcaccctcgtaggacgagcgccactcggccttgtgcttcttgtgcttcttgcccatggcggcgccggcggcgggcccgaggcgggggctgggaacagctggcacccggtcggaccttggc

SEQ ID NO: 439 - DNA, 122 bp, Homo sapiens
  source 1..122: mol_type = genomic DNA
ggtggcgcgcattcgcccactcggagaccgagaggcaggttttctgcctgcacagcctcccgcccaaggccagacctgctggaggcccaggccctggagatggctgtcttcagggagactct

SEQ ID NO: 440 - DNA, 122 bp, Homo sapiens
  source 1..122: mol_type = genomic DNA
gtcgtgtgctttgggctcaacgacatggctcggcgggcagccaggtaggggggcctggggcgcgcgccggcaggacgcggcggccgctcgtcgccgccacctcggccgcggcagctgagcct

SEQ ID NO: 441 - DNA, 122 bp, Homo sapiens
  source 1..122: mol_type = genomic DNA
gtcctctggtccttctttctgtctgtgcctccgtctttgtctcaacctctcaggcttgctcgctccctgcccagattttgtggcccaggctcctggctgtctgactccgggtttctgtcccc

SEQ ID NO: 442 - DNA, 122 bp, Homo sapiens
  source 1..122: mol_type = genomic DNA
gaggggaggggagagagttgggcgagggagagcccccggccggctgccagaagatcccggcgggaggaagcccaagtgtcacttgaattccacccaaggagcgggcgcctgggatcagagcg

SEQ ID NO: 443 - DNA, 122 bp, Homo sapiens
  source 1..122: mol_type = genomic DNA
tggaaagagaggcagggcccacggacaaacagactgggatggatgcatagacagacggatcgatcgggtggatgggctcacttgcaagtgcgctcgcggccaccggcgtggcagtcgaagtt

SEQ ID NO: 444 - DNA, 122 bp, Homo sapiens
  source 1..122: mol_type = genomic DNA
ggagccaggggcaccggcggagacacgagcgcagaaggcacgcgctcaagcccacgcccgcgactgccgggactgaaggtgttgcgagccccggctccacccctagcctgcggtgcgccgtg

SEQ ID NO: 445 - DNA, 122 bp, Homo sapiens
  source 1..122: mol_type = genomic DNA
tgtaacgtgttagtcatccattatctgcttctatttttgcaggttctgaatgatgactgacgcgggtttgggtgatacccctcacagcccctgtcattccggagtcataaggcacccgcgcg

SEQ ID NO: 446 - DNA, 122 bp, Homo sapiens
  source 1..122: mol_type = genomic DNA
ctaggagtgcataggcaagaatgtctgctgctcacagagatggtggcctggctgggtctgcgctaggctgccctgttctgccttctatcctctagtctttgttccttcaactgatatttcct

SEQ ID NO: 447 - DNA, 122 bp, Homo sapiens
  source 1..122: mol_type = genomic DNA
ctccaaagacccatcctcccaggcagccttccaggctgatcactgtgcctccaactccgtcgttcctgttccgatccccatcacgggctgagggtgaccgtgtttgtttatgaccgccccct

SEQ ID NO: 448 - DNA, 122 bp, Homo sapiens
  source 1..122: mol_type = genomic DNA
gtctagactctggatctctatttttaggtcgaagtgctttattcttgactcccgaattcccggaaactattaccaaagcagcttagttttctctccaccctgcctgggtcacaaatatgacg

SEQ ID NO: 449 - DNA, 122 bp, Homo sapiens
  source 1..122: mol_type = genomic DNA
ctcagatccaagattctgggtacccaggccttcttcctctaccactcagaagtggggtaccgaggcttcttctcccttagggacccaagactcctggcctcaggccctcctccctgagaccc

SEQ ID NO: 450 - DNA, 122 bp, Homo sapiens
  source 1..122: mol_type = genomic DNA
ggcggcccagcagggcagggccgggcacctggcaggtggggtcatccccacagggcaccccggccgagggcagagctggttccgccgcagggtcgatcctgggctctgggctgtgcacacgt

SEQ ID NO: 451 - DNA, 122 bp, Homo sapiens
  source 1..122: mol_type = genomic DNA
gctccactgatccggcccccgggggtgggcgggcacgtttgcctcggatggtctaacaggcgtcggggagagccaatggtgtggcttcatggctccacccccttccattcgattggccggcg

SEQ ID NO: 452 - DNA, 122 bp, Homo sapiens
  source 1..122: mol_type = genomic DNA
ggccctccggactgacgcggcctgagcagcagcgagtgtgaagtttggcacctccggcggcgagacggcgcgttctggcgcgcggctcctgcgtccggctggtggagctgctgcgccctatg

SEQ ID NO: 453 - DNA, 122 bp, Homo sapiens
  source 1..122: mol_type = genomic DNA
aaaaacaaactagctatggaagcaggaagtgaggtcaggctggagttgcagggactgtcccgggtgggttacaaaagcaggacctgcttagactatagacaggtgtgtgtgtgtgtgtgtgt

SEQ ID NO: 454 - DNA, 122 bp, Homo sapiens
  source 1..122: mol_type = genomic DNA
gaccgactgtcaaggtttatggtcgttgagggaatgccgtgggaatcagtggttgagaaacggggtaggaaagtgtgtatcagtgaatgaacttggtgtccattccgggtttgcaatttaat

SEQ ID NO: 455 - DNA, 122 bp, Homo sapiens
  source 1..122: mol_type = genomic DNA
tcctaagcctctctgagctgggcttggccaccttccggggtgtgagcgtccacgggagatcgaccacaccaggcacccaggagcaagtgctttgaaatgcggctttctccggaccttgcagg

SEQ ID NO: 456 - DNA, 122 bp, Homo sapiens
  source 1..122: mol_type = genomic DNA
gtgcagggaaagcacaccgtggctgcagcccagcaactggcagtaggtattttcaatggtcggcaggtactcatgacggaagttgccgctcgcccacttgtgcagcagcgtacttttcccca

SEQ ID NO: 457 - DNA, 122 bp, Homo sapiens
  source 1..122: mol_type = genomic DNA
taagaccacttgctgctccctggaatgattctaatataggaggtacattagagagagtgccgtaagaatagcctatattaaaaagaactaggtatgtagcttttaaagtgtgcccatttaga

SEQ ID NO: 458 - DNA, 122 bp, Homo sapiens
  source 1..122: mol_type = genomic DNA
gggctggaggcaggagggatgctccctggcaacctcacagggtgccgtcacatgacagggcggacacatgacagaattataataaaatgcttaagaagagtaagcaacagcatcttcagatt

SEQ ID NO: 459 - DNA, 122 bp, Homo sapiens
  source 1..122: mol_type = genomic DNA
tccctagggcttttcctgtactagaaagggaaactttgctttttttttttttttttgagacggagtcatactctgtcgcccagcctggtgtgtgtgcagtggcgcaatcccggctcactgaa

SEQ ID NO: 460 - DNA, 122 bp, Homo sapiens
  source 1..122: mol_type = genomic DNA
aaatgccttggccacaaggagaaaacattcgctggctgtggcttgagccaactgggtcagcgccagcctgtgcagatgtctttgtggacagagaagaagtccctctgggagaaggcagccca

SEQ ID NO: 461 - DNA, 122 bp, Homo sapiens
  source 1..122: mol_type = genomic DNA
cctcgagtagacaggtggatgtgagaaggattctcctttctggtgatttgcacctgttgacgcccccaaatctgagctctaaggaattttacagatggttaagacaggttctgaaaggtaga

SEQ ID NO: 462 - DNA, 122 bp, Homo sapiens
  source 1..122: mol_type = genomic DNA
tgccccactgacagtcacgctgcgaaggcagctctggccaataccagaaggatgggcaaacgcaacattggcatggatgcttttgctacaccgaattttgtttttctgtaatgggggaaaaa

SEQ ID NO: 463 - DNA, 122 bp, Homo sapiens
  source 1..122: mol_type = genomic DNA
tgggccattggtcagtctagcctgagggcgggttgttgggcggaagagagagacttcttccggcctcactcgctgtcaccatagagattgcccatccaggcagcgaagcagcagggccaggc

SEQ ID NO: 464 - DNA, 122 bp, Homo sapiens
  source 1..122: mol_type = genomic DNA
acaggacaagctctgaattgcattccaggaaaaggggactgaggctttgtatttttcctccgcaccaagattcccaaggctgctgttaagggtttttacccagggtggggtccacggcgaag

SEQ ID NO: 465 - DNA, 122 bp, Homo sapiens
  source 1..122: mol_type = genomic DNA
tttcaagccctaggtaaaagtgtgtcctgcctcgttactgggaagcaccatccacacacacgagcctacccagcctggggccctgtgtgccagcacctactctttttttttgagacggagtc

SEQ ID NO: 466 - DNA, 122 bp, Homo sapiens
  source 1..122: mol_type = genomic DNA
ctcttccgatagttctggcagagacggctgcggggagacacgggcggaggcgggtccgggcgggccccacgtcgcgcagcctcagcgtgttgcgcctcccgcggctgcggccagagacttcc

SEQ ID NO: 467 - DNA, 122 bp, Homo sapiens
  source 1..122: mol_type = genomic DNA
tggcacgacaacccagagggaggaagacctttccagtaggttttagaaaacatcgtgaaccggaattcagtggtcacctgaaaggcacatttcacagaccagctagcaaacagactcagcag

SEQ ID NO: 468 - DNA, 122 bp, Homo sapiens
  source 1..122: mol_type = genomic DNA
ttcacttcgtatttttagttcccaaaccttgacatccaagctcccgctctgtggcttccgcggcagcctggcaagcttcctgtgaagtcgcccgctgctgagcttccgcacccttcctcccg

SEQ ID NO: 469 - DNA, 122 bp, Homo sapiens
  source 1..122: mol_type = genomic DNA
aggtcagatactgtctgcctgatgtagcaattctctcaggctgtatctgcgataccagtgcggccggtgaagagggcgatggtgaattgcaccaataatgctgtaggtggcgctgtgtactt

SEQ ID NO: 470 - DNA, 122 bp, Homo sapiens
  source 1..122: mol_type = genomic DNA
ggaggcgggacaaggcttgcttgcgtcctccgtagattggcaggtcactgggacggccagcgcgtgcgcactggcctgtcagcggccggtggaccatggaggccgcaaggcccttcgcccgg

SEQ ID NO: 471 - DNA, 122 bp, Homo sapiens
  source 1..122: mol_type = genomic DNA
cagggggtcccggggacccacggggtgggtgggggctgcgctcaccttggcctcctgcaccgcctcgtccagcggcagcacggcgtgctcgcggtgctcgcgggcgcggtcgcacaccacgc

SEQ ID NO: 472 - DNA, 122 bp, Homo sapiens
  source 1..122: mol_type = genomic DNA
actcccatatgctgggccttcagtacgtttgtctttatattgggagtcctctgtggcctccgctgagggcgctggggcgggggttggggttggtggccagctgagcctggttgaagggcggc

SEQ ID NO: 473 - DNA, 122 bp, Homo sapiens
  source 1..122: mol_type = genomic DNA
caacagggacagaaggcaggtcccagaaagcaggttcccccaaaactggcttccctagcacggagttaaggctgcagccggctgcctagagagaagggtgggcagggagacaacgcggtgag

SEQ ID NO: 474 - DNA, 122 bp, Homo sapiens
  source 1..122: mol_type = genomic DNA
ctgactctcaggaggaccaagcattcttgggggagacaatttaaactaccaaggtacacccgcaactccctctcggagtgcccttacgttccttcggctggcccctcgccccaaaggagccc

SEQ ID NO: 475 - DNA, 122 bp, Homo sapiens
  source 1..122: mol_type = genomic DNA
ttcccctccacctttctccatagcaacggggtcattcctccctgaggtccaggagaagggcgacctctgccagcccaagaagcggtgagaactacattacccagaggcccgtgagccaggtt

SEQ ID NO: 476 - DNA, 122 bp, Homo sapiens
  source 1..122: mol_type = genomic DNA
taatatacgtgataacttacacaatgacgttcaacaaatacttgtggagtgaaagatgcgcgattcattgacagatctcagggatttcagaaagctgtataaggaccagctcttcaggctcc

SEQ ID NO: 477 - DNA, 122 bp, Homo sapiens
  source 1..122: mol_type = genomic DNA
gaatttacctgtgaaagtggataagtcccctgtagcgtgcttgcacagatggccataccacggatgcagcagtgattagccatgtaaactcactttctgcagttttaccagcatctacctct

SEQ ID NO: 478 - DNA, 122 bp, Homo sapiens
  source 1..122: mol_type = genomic DNA
cccgtggacctccacgtggtaggtggtgccgggcctgaggtcgggcaggctgacggtgcgcgtggtgcccggcacagtcagctcaccgccggggccctctgcaggcggctgaggccgccagc

SEQ ID NO: 479 - DNA, 122 bp, Homo sapiens
  source 1..122: mol_type = genomic DNA
tgttttcagtgtggtctcagagtgaaggtttctatacagggtttagtctgagaaacaactcgggaaacagatgaaaggcatttcagatgattaccctagagacacagtggttctcaagactt

SEQ ID NO: 480 - DNA, 122 bp, Homo sapiens
  source 1..122: mol_type = genomic DNA
tatgctggaacagcacattaaatggatccccttggtcagaggtcccagagggggcccaggcgacttcctctcaaaggacatgagtagtcagacctgccttccatttttttctgcccttacct

SEQ ID NO: 481 - DNA, 122 bp, Homo sapiens
  source 1..122: mol_type = genomic DNA
ccatggcgaagtggcggaggtgagcacctagaggcgaccctgcccggggaacagctggcgcgaccgcggacagagcttcccaccacgcccttccccgcctttggccagcctttgccgtatgt

SEQ ID NO: 482 - DNA, 122 bp, Homo sapiens
  source 1..122: mol_type = genomic DNA
tcagggtcagagtgagcagaagccactcatcctgaccgaagatgacgacgaagatgacgacgtcccagagggggtggagcgtgtgataggtgcgtggggtctaagcggcggcctctgctctt

SEQ ID NO: 483 - DNA, 122 bp, Homo sapiens
  source 1..122: mol_type = genomic DNA
gctgtgtgcgtctgagcttcggaggcagcagggtctgaacattttggactcaatgctgcacgactcggccctcccagctttgcaccagggagcagctgcgaactttggggctcctcggagaa

SEQ ID NO: 484 - DNA, 122 bp, Homo sapiens
  source 1..122: mol_type = genomic DNA
ttgacccagggccagggtctctccgcctttgcggggaaggggtggggaagtgtgtaggagcgtgggtaatttgggtaggaaaaggcagggagcaccctaccagccctcctaggatacaggct

SEQ ID NO: 485 - DNA, 122 bp, Homo sapiens
  source 1..122: mol_type = genomic DNA
tctccccagactccagactctagagggcgacctcctcctgctcctgctcctggagcgcagcgagcgcacagcgtttccgcaggaatcctgagaatggcaaggcccccatacccgcggtggtt

SEQ ID NO: 486 - DNA, 122 bp, Homo sapiens
  source 1..122: mol_type = genomic DNA
ggtcttgtcagctctcttgatatataatgtactaattcaagcggttcaattccttgttgacgataatgatacaatacatgtattgagtacttaaaagccactgacgttctgtctgcaggggg

SEQ ID NO: 487 - DNA, 122 bp, Homo sapiens
  source 1..122: mol_type = genomic DNA
tagaacagcaggacctgcgaaactctgaggccgctttgtgaggtcctcctctgcgcagcacgccccccacccctctcttggtgccgccgcagctactccctagggggctttgctcttggtgg

SEQ ID NO: 488 - DNA, 122 bp, Homo sapiens
  source 1..122: mol_type = genomic DNA
gaacctaaatccagtgatggaatcttattgtccacagtcttttgtggtatgtgctaccaccgaagatcctctcctaacctatcatgtgttcaatacgtctcagctcctagacttgctgaagc

SEQ ID NO: 489 - DNA, 122 bp, Homo sapiens
  source 1..122: mol_type = genomic DNA
aaggcggcggcggcggcgaaccagcagaagggactttcctggcagcccggcgacgaggagcgcggacagtgagtttgctctgccccggttcatggttcctgcaagccctctaggaggccgaa

SEQ ID NO: 490 - DNA, 122 bp, Homo sapiens
  source 1..122: mol_type = genomic DNA
gagaaacttgtcaacgtcacttgggcatcttaagagtgggttcgtaaacttggttgtgtgcgctgtgcagatgtcagtcaccctgtgtggtgggcaaagccgacttctccgcctctgtagct

SEQ ID NO: 491 - DNA, 122 bp, Homo sapiens
  source 1..122: mol_type = genomic DNA
cccctcccctctgtgcccacaggtgtcctccctgggcagcggcagtgaccacgtcatggacgccatctcccagtgcgagcagtacgccaaggagcagggcgcccaggagcgcaacgccccct

SEQ ID NO: 492 - DNA, 122 bp, Homo sapiens
  source 1..122: mol_type = genomic DNA
agccccatttaagcagtgcctcgggatttgagttgcctgtgaaacgcagcagccttctgccgaggacgcagaaacccccgcctctcatccagggctgacagggcacgggccgcgagccgcgg

SEQ ID NO: 493 - DNA, 122 bp, Homo sapiens
  source 1..122: mol_type = genomic DNA
ataagagaataaaagcagggtgcctcaggtagaagtggcaatctgctcggtcgtcttccacgctgtggatgctttgttctttcactctttgcaataaatgttgctgctgctcactctttggg

SEQ ID NO: 494 - DNA, 122 bp, Homo sapiens
  source 1..122: mol_type = genomic DNA
atgttagattcaccccacagagatagcggcagagctggcagcggacggtctttgcattgccgcctccccagggggcgggaagctggtaaggaagcagcctgggttagctaggggtggggtca

SEQ ID NO: 495 - DNA, 122 bp, Homo sapiens
  source 1..122: mol_type = genomic DNA
tttctcccagcaagtttacttgtaagcatctccatatactgttgtaaataatgatggtcacgtagtatcagtggctgtatgctgaactctctgcactgacctaccggcagacagcaacctga

SEQ ID NO: 496 - DNA, 122 bp, Homo sapiens
  source 1..122: mol_type = genomic DNA
gccatgtgtaggggagagggaggttcctgggttggccacagcccccatggtggtacagaacgcatgcagggcggttgccctgtcctttgaagactgcacactgtgtgtcaggcagccctctg

SEQ ID NO: 497 - DNA, 122 bp, Homo sapiens
  source 1..122: mol_type = genomic DNA
ccaggtggctgcccttccaagtcccacactcttgtcctgatggcctccgaccccgggcctcgagcccaaccaaaggcaccgaaggagagaaaagccaactcactagggtgccctctccagcc

SEQ ID NO: 498 - DNA, 122 bp, Homo sapiens
  source 1..122: mol_type = genomic DNA
tcgagccacgctccacttcccgggaagaattctggggagagtgaaggctcgctctgtggccgccccgcccacagcccgcctggggaagccgcgggccccagctcacctgcaggttgctcacg

SEQ ID NO: 499 - DNA, 122 bp, Homo sapiens
  source 1..122: mol_type = genomic DNA
ggctctgcaccaaggaaggctgaaccgagagaaccttggactagaggtcgtggtactcagcgctcccttcactgaccatttaaatgtaaagcaatgtttgtcctcgctgtcagtcgcaacac

SEQ ID NO: 500 - DNA, 122 bp, Homo sapiens
  source 1..122: mol_type = genomic DNA
aaagcatagtggatttagtctaacattttcccacctggaaaaactgggcttaagtatctccgacaaacaaaaagcaatactgcgttcttcccaatatgccatgtcacctttgtagcactcac

SEQ ID NO: 501 - DNA, 122 bp, Homo sapiens
  source 1..122: mol_type = genomic DNA
cagacgccgcatggaatgcggggccggctccactccttccgtaatgtggttttttatagtcgggggactcattgtcccgtggccactgccagctgtctgtaagctcaggattagagagcctg

SEQ ID NO: 502 - DNA, 122 bp, Homo sapiens
  source 1..122: mol_type = genomic DNA
gtttcactgcgtcttcaccacaaggacagccactggctccgtctttagatgggaaaactgcggcccggcgacttttgctagttggaaaggtgggtcagtggtggaattaagtcacacttagg

SEQ ID NO: 503 - DNA, 122 bp, Homo sapiens
  source 1..122: mol_type = genomic DNA
ggaatggggggcgggtgtcttgccacttgtgtgacaggcttaacctttttgtatgaagttcgtttgccttatcggccttactgtttgatagtttactgtgtctgatttcttcccccgtactt

SEQ ID NO: 504 - DNA, 122 bp, Homo sapiens
  source 1..122: mol_type = genomic DNA
tggctgacagctgcctccaaacacccagaggagcacatcagaatctggtttgccacccagcgcttaaagcatggcatcagctggtccccagaagaggtggaggaggcccggaagaagatgtt

SEQ ID NO: 505 - DNA, 122 bp, Homo sapiens
  source 1..122: mol_type = genomic DNA
gataagcgcctaatatacatccctgcctgtcattattcacattgtggcatgcagtcaaagcgacactctgaggaaaatgtatcgccttaaatacattgattagaaaataagaaagcccgaac

SEQ ID NO: 506 - DNA, 122 bp, Homo sapiens
  source 1..122: mol_type = genomic DNA
gtggttttgacggttgggccgtgtgagttgctaggactcacctgggtctccagtcagtgccgggctgccgcccctgccgccgccgccgccgcccctgccgccgccgccgccgccgccgcccc

SEQ ID NO: 507 - DNA, 122 bp, Homo sapiens
  source 1..122: mol_type = genomic DNA
tttccgctgcacccctacctacccaatttttatctcgctacaatcaggggttttctcaaccggggaaaaggtggtagtggcggtgggagggtcaggatccctcgaagtggaggccggggccg

SEQ ID NO: 508 - DNA, 122 bp, Homo sapiens
  source 1..122: mol_type = genomic DNA
aatctacatgtctaataacagcaaatctgctaagaagcattagaaagaggagcagcgccccggaatctctgcaggaaatgttctttatattagacatatacaagcaggtcctgtcagatgca

SEQ ID NO: 509 - DNA, 122 bp, Homo sapiens
  source 1..122: mol_type = genomic DNA
acagcctgggctcaacctctcccagacttcctggactccagtggagccttgggccgggggcggggcgtccctggcccctccccgtcgtcccgcctgcccggaaaggagtgagcggcgcctag

SEQ ID NO: 510 - DNA, 122 bp, Homo sapiens
  source 1..122: mol_type = genomic DNA
aagcaccaaactaggcagctggataatgggagagtcggctggttgtgagcatgctcgcgccgggaacagatccaccctctgttattcctctgaataaatataaatcacgatcagaaacccaa

SEQ ID NO: 511 - DNA, 122 bp, Homo sapiens
  source 1..122: mol_type = genomic DNA
gttgcggggctcggggaagttacccccatccgtgctggagtagcggggaagccctgggtgcgttacactcgaccgtgatggggagaggggacttagatgttgtcacgctgggggtcccttta

SEQ ID NO: 512 - DNA, 122 bp, Homo sapiens
  source 1..122: mol_type = genomic DNA
ctgcgatcttcccgtgcctgaatatgaggcttggaacagacccagaccttcctgcctgcccgtcctgagtggccccgggaccccgccccatctttggcccccagcccctgcctctctgccgc

SEQ ID NO: 513 - DNA, 122 bp, Homo sapiens
  source 1..122: mol_type = genomic DNA
cttgggcaacgtaggagacctccgtctccacaagtaaaattaattagccggctgtggtggcgcgcacctgtggtcccagctactcaggaggctgaggtaggaggatcacctgagcccgggag

SEQ ID NO: 514 - DNA, 122 bp, Homo sapiens
  source 1..122: mol_type = genomic DNA
ttaccttggtggcccccgcttcctccccacggagcccggcgctcacccccgggccggggtcggggtcgtgagcccacagctcagccaccatctccgtgtgaagaaactcaaacaacacagta

SEQ ID NO: 515 - DNA, 122 bp, Homo sapiens
  source 1..122: mol_type = genomic DNA
ctggcagagggaaagggattttttttttctccctccctgccgaggaaaaaacagttctaacgaggagttactttgtagttttaactcatattcaaatactcccagccgagaagctgccatcg

SEQ ID NO: 516 - DNA, 122 bp, Homo sapiens
  source 1..122: mol_type = genomic DNA
agccctggtcactgagacaaaagatacagagacaccgggacacagagaggtacaacgggacgtggagttggatcgagctgtctcgtaaggaatatcggaaggggtggggacactactgggag

SEQ ID NO: 517 - DNA, 122 bp, Homo sapiens
  source 1..122: mol_type = genomic DNA
gcccctcaaccctcctggacccaactgtgcccccgcttagcttccagtccagaccttccccgcaaatgagtgtgtgctgtgagtgagaccccgcgtgtctgcccttgcagtccgccctgagg

SEQ ID NO: 518 - DNA, 122 bp, Homo sapiens
  source 1..122: mol_type = genomic DNA
gcgggggcttagtctaggcccggcagggttttctggaagaccagagggccaccaggtcaccgaggtgggaagtgaagagaggttcgacgctgcctcaggcctgggcctggccggtgggagac

SEQ ID NO: 519 - DNA, 122 bp, Homo sapiens
  source 1..122: mol_type = genomic DNA
agggagcggccacccagcccggggcctgcaagcaggcaggcagccattcgccagcagccccgggcccgggctgactcactgggggccccctgctgtggcctggaccctcacgctatcccggg

SEQ ID NO: 520 - DNA, 122 bp, Homo sapiens
  source 1..122: mol_type = genomic DNA
tggctgccgggggcgggaaagtgatttctcggaaagcagagcacttcgaagaaggcgggccgcgcgagccaagctgacgctattggtcggtgtggccgtcgctctgcgcaccgcccgtcccc

SEQ ID NO: 521 - DNA, 122 bp, Homo sapiens
  source 1..122: mol_type = genomic DNA
caactgccccgacccccagaagtgcaaaacgaacaggctggcaagtgaccaaaagagacccggggagcatctgggcttccaaggtcctcggtacggcccaaggcagcgaaggacgcgcggct

SEQ ID NO: 522 - DNA, 122 bp, Homo sapiens
  source 1..122: mol_type = genomic DNA
ggtctccactgccctccaggctccatccacccgcctggtttcccgggtcgctgtggcccgcgctggcgctgctgttgatctgctgtgtgtgctgttcccgaagaggctccgggagcctgagc

SEQ ID NO: 523 - DNA, 122 bp, Homo sapiens
  source 1..122: mol_type = genomic DNA
gcacacagaaaagaccaatcaaggacgggtcattcccgccccccgcgcgccttttgcgaccgcccactcgacaggttgacaacctagacagctcccccggacttgccttacttttccatctc

SEQ ID NO: 524 - DNA, 122 bp, Homo sapiens
  source 1..122: mol_type = genomic DNA
ctgaaccggagatccagaaggaccctccagggatgacctcccaactctttgcctagagatcggcaagcacgttgccggaggctctgtccagaacatccgctctgcggctcacttttcatggt

SEQ ID NO: 525 - DNA, 122 bp, Homo sapiens
  source 1..122: mol_type = genomic DNA
aataagtcattataaatagttgctccgtgggctttccgcatttcaccctgctccaggcagcgtggtctgtatggctccgccctgggcaggaggagaaagaagagggccaggcaccacgtgcc

SEQ ID NO: 526 - DNA, 122 bp, Homo sapiens
  source 1..122: mol_type = genomic DNA
gtgatatatgtgaaataatccggacatgcttctgctatggttgtttgtcgccatagtttgcgaatctgggtaaacctggatgagagagacgccttacaattgcaaacatttctcaggaggcc

SEQ ID NO: 527 - DNA, 122 bp, Homo sapiens
  source 1..122: mol_type = genomic DNA
ggatgtgcataactgaagctgcctgaggactggaggactgaccggggccagcacagcgggcgggggcctgaggactcgaggactgaccggggccagcacagcgggcggggctctgcccatta

SEQ ID NO: 528 - DNA, 122 bp, Homo sapiens
  source 1..122: mol_type = genomic DNA
ctgcccttcgcttggtctagttcgtgctcgccaaacctgccaccgttttgtgtctgctcacggaacgtgatctctctatacgcgttaagacgtttgatttggttcttgttctgcttatggaa

SEQ ID NO: 529 - DNA, 122 bp, Homo sapiens
  source 1..122: mol_type = genomic DNA
cggaggcccggggtccagggcgccccctgctgggaagctgggaaccaagccttagaggtccggcaggtacagatggaaccaggcccagggcgacccctcctagggaaaccaagggtttctag

SEQ ID NO: 530 - DNA, 122 bp, Homo sapiens
  source 1..122: mol_type = genomic DNA
taaatgccgcgggggtggcgcgcggggagtgggtttgggcaccctcctcgccctcccgcgcggaaaaactggcacatagatgccgcctgactcctccaaagccattagaagatttggggtgg

SEQ ID NO: 531 - DNA, 122 bp, Homo sapiens
  source 1..122: mol_type = genomic DNA
acattccagcctgggtgacacagcaagacctggtgtcaaaaactaaaaaccatctcgaatcggcgcccccgccacaagcattttttctcgtcggcagctcagttttcagctgtgcttagtct

SEQ ID NO: 532 - DNA, 122 bp, Homo sapiens
  source 1..122: mol_type = genomic DNA
gcagcgctcgccgcgcctctagtgggagcctctggcctggtggtttccggggagagagcccgaagagcaagggcctcggcagcttcctcagtggggcagggccggcgatgccagccagggac

SEQ ID NO: 533 - DNA, 122 bp, Homo sapiens
  source 1..122: mol_type = genomic DNA
tcctcttctttgtgagaaaaggatgaatctttcctgatttactgttgcctcttaaacacccgtggcaggaatctttctcacaccaggggcttctgtgtcatgctgatatgcctggaactagc

SEQ ID NO: 534 - DNA, 122 bp, Homo sapiens
  source 1..122: mol_type = genomic DNA
accatgccaaacctggccgggacccgctcctgatcccttcacatccacggattcccccagcgccccccaccccagggaactcctgctaatcaaccaagctgaaacctggaaaaaatcaagcc

SEQ ID NO: 535 - DNA, 122 bp, Homo sapiens
  source 1..122: mol_type = genomic DNA
cagcccctcgcgcgccgaggccgccggagccggggtggccgcgagaaagcagggaagccgcgcgctgcaagctcaagcctcgccgacgctagccccgaacacaaagcgagcgcccgcgtccg

SEQ ID NO: 536 - DNA, 122 bp, Homo sapiens
  source 1..122: mol_type = genomic DNA
cgttaccatgacgaccgggctcctagaagctgcagtcaaggacctggttgccatggtttccgcttctccgcctccagccccgcccgcgctccccgcggcgtcggcgcctgcgcagtgcgtgg